(19) United States
(12) Patent Application Publication
(10) Pub. No.: US 2025/0197845 A1
(43) Pub. Date: Jun. 19, 2025

(54) COMPOSITIONS AND METHODS FOR IMPROVED DNA-ENCODED LIBRARY PREPARATION

(71) Applicant: The Rockefeller University, New York, NY (US)

(72) Inventors: Marc Flajolet, New York, NY (US); Yashoda Sunkari, New York, NY (US); Thu-Lan Nguyen, New York, NY (US); Vijay-Kumar Siripuram, New York, NY (US)

(21) Appl. No.: 18/849,817

(22) PCT Filed: Mar. 21, 2023
(86) PCT No.: PCT/US2023/064758

§ 371 Date: Sep. 23, 2024

Related U.S. Application Data
(60) Provisional application No. 63/322,792, filed on Mar. 23, 2022.

(51) Int. Cl. C12N 15/10; C12Q 1/6806; C40B 30/04

(52) U.S. Cl. CPC C12N 15/1065; C12N 15/1068; C12Q 1/6806; C40B 30/04

(57) ABSTRACT

Disclosed herein are compositions and methods for detecting oligonucleotide molecules that can be ligated with high efficiency, and methods of using the oligonucleotides to tag DNA encoded libraries and to modify existing DNA encoded libraries to incorporate new functionalities. Also disclosed are compositions for increasing DNA solubility in non-aqueous solvents and assay systems for detecting compounds or conditions that increase the solubility or durability of DEL.

### CROSS REFERENCE TO RELATED APPLICATIONS

This application claims priority to U.S. Provisional Application No. 63/322,792, filed Mar. 23, 2022, which is hereby incorporated by reference herein in its entirety.

### BACKGROUND

Large-scale technologies such as DNA-encoded library (DEL) screening or DNA assisted phage display screening are increasingly ambitious both in term of scope and size, in a number of fields ranging from drug discovery to medical sciences. These technologies require complex encoding (e.g. DNA barcode) and their successful use is largely dependent on their overall quality. It is now frequent to hear in the DEL field that the most difficult part is to identify the right compounds among the background at the end of a screening campaign. The field is largely dominated by chemists focusing their effort on generating novel molecules, on discovering new chemical reactions. However, little attention has been given to improving the DNA tagging, the DNA tags themselves, and all molecular biology aspects in general.

A DNA barcode is the result of assembling a number of smaller DNA fragments (e.g. DNA tags). Once assembled, different DNA fragments create what we can call a DNA label, and each molecule in such a library is tagged with a unique and specific DNA label.

Within this DNA label, the DNA tags that track another property (e.g., chemical blocks) are obviously extremely important and are defined by a number of parameters that impact on the overall quality of the library; other DNA fragments also enter in the composition of a full DNA label. All these DNA fragments are added successively one to another in a specific sequential organization by DNA ligation.

The presence of the various DNA fragments within a DNA label, their specific sequence, their order and exact spacing, and their relative abundance, etc., are crucial parameters necessary for the entire approach; they also have a great impact on decoding steps, on identifying the hit compounds at the end of a screening campaign.

A DEL is a mixture of large numbers (millions to billions) of drug-like molecules of small molecular weight, where each molecule is conjugated to a specific and unique DNA-barcode that encodes its chemical structure. The composition of a DEL mixture can be readily interrogated before or after interacting with a protein of choice following a PCR amplification step and Next Generation Sequencing. The identification of DEL labels associated with the protein of interest allows to deduce the structure of their corresponding molecules, of the hit molecules that selectively bind to the target. A typical DEL molecule is a hybrid molecule harboring a DNA label linked with a chemical linker to the small molecular weight molecule that was generated by serial addition of building blocks (BBs) onto a scaffold, using a combinatorial approach that most often is based on a split-and-pool protocol. The combinatorial approach gives rise to libraries containing millions to billions of compounds.

This concept commands that a number of organic chemistry steps happen in presence of DNA, the base of the DEL label and as the process progresses, short double-stranded DNA tags encoding each building block are being added by DNA ligation onto the growing DEL label.

Nucleic acids, and DNA in particular, are highly polar due to the presence of negative charges decorating the DNA strands. Each nucleotide harbors one phosphate group that is bringing one negative charge. Therefore, the DNA phosphate backbone is negatively charged due to the presence of bonds created between the phosphorus and oxygen atoms. These nucleic acid phosphate groups create an overall polar backbone that has a pK near 0. They are fully ionized, negatively charged at pH 7.0, which qualify them as acid molecules. Furthermore, the hydroxyl groups of the sugar residues form hydrogen bonds with water. Altogether this means that DNA is hydrophilic and therefore soluble only in aqueous solutions. Because a large number of organic chemistry reactions are happening in organic solvents and cannot be carried out in aqueous solution, the DEL technology and its chemical space has not been much explored due to this important limitation. Reaction compatibility in organic solvents: DNA is insoluble in anhydrous organic solvents whereas chemical building blocks are mostly not soluble in aqueous medium remains a major challenge for DEL technology.

For generating, structurally diversified DEL molecules, novel chemical transformations in aqueous solution in presence of DNA must be developed. Due to the idiosyncratic nature of the DNA, the chemical transformations that can proceed in presence of DNA are quite limited compared to traditional medicinal chemistry that often requires harsh conditions. Development of on-DNA compatible reactions are restricted by the chemical reactivity of DNA, particularly low pH conditions that easily degradation of DNA through depurination. DNA is also labile at high temperatures, and highly sensitive to some reagents such as metal catalysts, strong oxidants and radical-based chemical transformations.

The fields of organic chemistry and molecular biology are largely intermingled in the fabrication of a DNA-encoded library but little is known about the interface of DNA biology and organic chemistry to address the compatibilities of DNA durability and DEL chemistry. It is well accepted that coupling chemistries in presence of DNA is limited due to the relatively fragile nature of DNA.

Accordingly, there is a need for improved methods for generating DEL libraries that are distinguishable, durable and soluble in organic solutions. The present invention fulfills this need.

### SUMMARY

In one embodiment, the invention relates to a system for identifying an oligonucleotide having high ligation efficiency, the system comprising:
a) a substrate for attaching a first accessory oligonucleotide;
b) a first accessory oligonucleotide comprising a moiety for attaching the first oligonucleotide to the substrate;
c) a second accessory oligonucleotide comprising a moiety for recognition by a detectable moiety;
d) a detectable moiety for detection of the ligation between the first accessory oligonucleotide, an intermediary test oligonucleotide and the second accessory oligonucleotide; and
e) at least one intermediary test oligonucleotide.

In one embodiment, the first accessory oligonucleotide is conjugated to biotin. In one embodiment, the second accessory oligonucleotide is conjugated to digoxigenin (DIG). In one embodiment, the detectable moiety comprises an anti-DIG-HRP antibody.

In one embodiment, the intermediary test oligonucleotide is a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, and an oligonucleotide comprising or encoding sequences for antibody recognition.

In one embodiment, the invention relates to a system for identifying an oligonucleotide having high ligation efficiency, the system comprising:
a) a substrate for attaching a first accessory oligonucleotide;
b) an accessory oligonucleotide comprising a moiety for attaching the accessory oligonucleotide to the substrate;
c) a test nucleic acid molecule comprising a moiety for recognition by a detectable moiety; and
d) a detectable moiety for detection of the ligation between the accessory oligonucleotide, and the test nucleic acid molecule.

In one embodiment, the accessory oligonucleotide is conjugated to biotin. In one embodiment, the test nucleic acid molecule is conjugated to digoxigenin (DIG). In one embodiment, the detectable moiety comprises an anti-DIG-Horse Radish Peroxidase (HRP) antibody.

In one embodiment, the test nucleic acid molecule is a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, an oligonucleotide comprising or encoding sequences for antibody recognition, a nucleic acid molecule conjugated to a molecule for binding or purification, a nucleic acid-antibody conjugate, a nucleic acid-nanobody conjugate, a nucleic acid molecule conjugated to a molecule for visulaization, a nucleic acid-fluorophor conjugate, a nucleic acid-quantum dot conjugate, a nucleic acid molecule conjugated to a molecule for cell permeabilization, or a nucleic acid molecule conjugated to a molecule for proximal PCR or rolling circle amplification (RCA).

In one embodiment, the invention relates to a method for identifying an oligonucleotide having high ligation efficiency, the method comprising the steps of:
a) contacting an intermediary test oligonucleotide molecule with a first accessory oligonucleotide conjugated to a surface and a second accessory oligonucleotide conjugated to a moiety for direct or indirect detection,
b) ligating the intermediary test oligonucleotide molecule to the first accessory oligonucleotide and the second accessory oligonucleotide,
c) removing any unligated oligonucleotides by washing,
d) contacting the ligated complexes with a detectable molecule for colorimetric, chemiluminescent or fluorescent detection, and
e) determining the ligation efficiency of the test oligonucleotide molecule to the first accessory oligonucleotide and the second accessory oligonucleotide by detecting the colorimetric, luminescencent or fluorescencent readout of the detectable molecule.

In one embodiment, the first accessory oligonucleotide is conjugated to biotin. In one embodiment, the second accessory oligonucleotide is conjugated to digoxigenin. In one embodiment, the detectable molecule comprises an anti-DIG-HRP antibody.

In one embodiment, the intermediary test oligonucleotide is a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, or an oligonucleotide comprising or encoding sequences for antibody recognition.

In one embodiment, the invention relates to a method for identifying an oligonucleotide having high ligation efficiency, the method comprising the steps of:
a) contacting an test nucleic acid molecule with an accessory oligonucleotide conjugated to a surface,
b) ligating the test nucleic acid molecule to the accessory oligonucleotide, c) removing any unligated oligonucleotides by washing,
d) contacting the ligated complexes with a detectable molecule for colorimetric, chemiluminescent or fluorescent detection, and
e) determining the ligation efficiency of the test nucleic acid molecule to the accessory oligonucleotide molecule by detecting the colorimetric, luminescencent or fluorescencent readout of the detectable molecule.

In one embodiment, the first accessory oligonucleotide is conjugated to biotin. In one embodiment, the second accessory oligonucleotide is conjugated to digoxigenin. In one embodiment, the detectable molecule comprises an anti-DIG-HRP antibody.

In one embodiment, the test nucleic acid molecule is a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, an oligonucleotide comprising or encoding sequences for antibody recognition, a nucleic acid molecule conjugated to a molecule for binding or purification, a nucleic acid-antibody conjugate, a nucleic acid-nanobody conjugate, a nucleic acid molecule conjugated to a molecule for visulaization, a nucleic acid-fluorophor conjugate, a nucleic acid-quantum dot conjugate, a nucleic acid molecule conjugated to a molecule for cell permeabilization, or a nucleic acid molecule conjugated to a molecule for proximal PCR or rolling circle amplification (RCA).

In one embodiment, the invention relates to a method of tagging a DNA encoded library (DEL), the method comprising ligating at least one oligonucleotide molecule to the DEL.

In one embodiment, the oligonucleotide molecule comprises a DNA blocker molecule comprising a restriction enzyme recognition site and a free chemical group.

In one embodiment, the method further comprises contacting the tagged DEL with a restriction enzyme for cleaving the tagged DEL at the restriction enzyme recognition site and subsequently ligating a at least one additional oligonucleotide molecule to the DEL. In one embodiment, the at least one additional oligonucleotide molecule is a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, or an oligonucleotide comprising or encoding sequences for antibody recognition. In one embodiment, the at least one additional oligonucleotide molecule is a DNA blocker molecule comprising a restriction enzyme recognition site and a free chemical group.

In one embodiment, the method further comprises functionalizing the DNA blocker molecule with a molecule to increase solubility in organic solvents.

In one embodiment, the invention relates to an oligonucleotide tag molecule comprising at least one nucleotide sequence of TCGGAGAA, TCGGAGCT, TCGGATAC, TCGGACGT, TCGGAGAT, TCGGAAGA, TCGGATTG, TCGGACAA, TCGGAGTA, TCGGAAGT, TCGGATCA, TCGGACAT, TCGGAGTT, TCGGAACA, TCGGATCT, TCGGACTA, TCGGAGCA, TCGGAACT, TCGGACGA, TCGGACTT, CAGAGGAG, CAGAGGTA, CAGAGAGG, CAGAGAAC, CAGAGGAA, CAGAGGTT, CAGAGAGA, CAGAGATG, CAGAGGAT, CAGAGGTC, CAGAGAGT, CAGAGATC, CAGAGGAC, CAGAGGCA, CAGAGAGC, CAGAGACG, CAGAGGTG, CAGAGGCT, CAGAGAAG, CAGAGACA, ACGTGGAG, ACGTGGTA, ACGTGAGG, ACGTGAAC, ACGTGGAA, ACGTGGTT, ACGTGAGA, ACGTGATG, ACGTGGAT, ACGTGGTC, ACGTGAGT, ACGTGATC, ACGTGGAC, ACGTGGCA, ACGTGAGC, ACGTGACG, ACGTGGTG, ACGTGGCT, ACGTGAAG, and ACGTGACA.

In one embodiment, the invention relates to a kit comprising at least one of:
a) a substrate for attaching a first accessory oligonucleotide; and
b) a first accessory oligonucleotide comprising a moiety for attaching the first accessory oligonucleotide to the substrate.

In one embodiment, the kit further comprises
c) a second accessory oligonucleotide comprising a moiety for recognition by a detectable moiety; and
d) a detectable moiety for detection of the ligation between the first accessory oligonucleotide, an intermediary test oligonucleotide and the second accessory oligonucleotide.

In one embodiment, the invention relates to a DNA solubilizer molecule comprising a DNA blocker molecule covalently linked to a molecule for modulating the solubility of DNA, wherein the DNA blocker molecule comprises a restriction enzyme recognition site. In one embodiment, the molecule for modulating the solubility of DNA is polyethylene glycol (PEG), methoxy PEG-succinimidyl carboxyl methyl ester, or methoxy PEG-succinimidyl carboxyl methyl ester with a N-Hydroxysuccinimide group. In one embodiment, the molecule for modulating the solubility of DNA comprises methoxy PEG-succinimidyl carboxyl methyl ester having a molecular weight in the inclusive range of 500 to 50,000.

In one embodiment, the invention relates to a method of identifying DNA solubilizer molecules that can alter the solubility of DNA molecules and DEL, the method comprising:
a) ligating a DEL DNA molecule, or a DNA molecule to be ligated to a DEL, to a DNA solubilizer molecule comprising a DNA blocker molecule covalently linked to a molecule for modulating the solubility of DNA, wherein the DNA blocker molecule comprises a restriction enzyme recognition site,
b) contacting the fused DEL DNA molecule:DNA solubilizer molecule with an organic solvent and testing the solubility of the fused DEL DNA molecule:DNA solubilizer molecule in the organic solvent. In one embodiment, the organic solvent is DMSO, DMF, DMA, 1,4-dioxane, ACN or DCM.

In one embodiment, the invention relates to a method of generating a DEL containing compounds that require an organic solvent, the method comprising:
a) ligating a DEL DNA molecule to a DNA solubilizer molecule comprising a DNA blocker molecule covalently linked to a molecule for modulating the solubility of DNA, wherein the DNA blocker molecule comprises a restriction enzyme recognition site,
b) contacting the ligated DEL DNA molecule:DNA solubilizer molecule with the organic solvent,
c) performing at least one chemical reaction to attach the DEL DNA molecule to the compound that requires an organic solvent, and
d) contacting the ligated DEL DNA molecule:DNA solubilizer molecule with a restriction enzyme to remove the DNA solubilizer molecule, thereby allowing for further ligation and labeling of the DEL DNA molecule.

In one embodiment, the organic solvent is DMSO, DMF, DMA, 1,4-dioxane, ACN or DCM.

In one embodiment, the invention relates to a DEL containing compounds that require an organic solvent, wherein the DEL is generated according to a method comprising:
a) ligating a DEL DNA molecule to a DNA solubilizer molecule comprising a DNA blocker molecule covalently linked to a molecule for modulating the solubility of DNA, wherein the DNA blocker molecule comprises a restriction enzyme recognition site,
b) contacting the ligated DEL DNA molecule:DNA solubilizer molecule with the organic solvent,
c) performing at least one chemical reaction to attach the DEL DNA molecule to the compound that requires an organic solvent, and
d) contacting the ligated DEL DNA molecule:DNA solubilizer molecule with a restriction enzyme to remove the DNA solubilizer molecule, thereby allowing for further ligation and labeling of the DEL DNA molecule.

In one embodiment, the invention relates to a method of identifying reagents or conditions that can alter the durability of DNA molecules and DEL, the method comprising:
a) obtaining a short double stranded oligonucleotide to be tested,
b) covalently linking the two strands of the short oligonucleotide molecule to be tested with a spacer that carries a free functional group for chemical addition and denaturing the dsDNA molecule to generate a linked ssDNA molecule,
c) contacting the linked ssDNA molecule of a) with one or more condition or reagent to be tested for its effect on DNA durability, and
d) analyzing the effect of the condition or reagent on the durability of the short oligonucleotide molecule.

In one embodiment, the condition or reagent to be tested is organic solvents, buffers, high temperatures, altered pH, metal catalysts, metal scavengers, % GC content, non-natural nucleotides, chemical ligands or any combination thereof.

In one embodiment, the method further comprises a step of precipitating the short oligonucleotide molecule prior to analyzing the effect of the condition or reagent on the durability of the molecule.

In one embodiment, the method of analyzing the effect of the condition or reagent on the durability of the short oligonucleotide molecule is performed using gel electroporation, LCMS, or a combination thereof.

In one embodiment, the invention relates to a DEL generated in a condition identified according to a method comprising:
a) obtaining a short double stranded oligonucleotide to be tested,
b) covalently linking the two strands of the short oligonucleotide molecule to be tested with a spacer that carries a free functional group for chemical addition and denaturing the dsDNA molecule to generate a linked ssDNA molecule,
c) contacting the linked ssDNA molecule of a) with one or more condition or reagent to be tested for its effect on DNA durability, and
d) analyzing the effect of the condition or reagent on the durability of the short oligonucleotide molecule.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts an overview of the method to effectively measure, optimize and calibrate small DNA fragment ligation efficiency.

FIG. 2 depicts a diagram demonstrating the different parameters of the method that are being optimized or tested.

FIG. 3 depicts experimental data demonstrating testing of the ligation efficiency of over 1,700 different tag sequences (7 nt long DNA fragments+3 nt overhang each side).

FIG. 4 depicts a diagram of the experimental design for testing of the ligation efficiency of DNA tag polynucleotides with different length nucleotide overhangs.

FIG. 5 depicts experimental data demonstrating the results from the testing of the ligation efficiency of DNA tag polynucleotides with different numbers of nucleotides in the overhang (OH) after only 1 hour of ligation.

FIG. 6 depicts experimental data demonstrating the results from the testing of the ligation efficiency of DNA tag polynucleotides with different overhang sequences.

FIG. 7 depicts experimental data demonstrating the results from the testing of the ligation efficiency of two different tag sequences (core sequences: GACTATC and GTGAGTT) extended on each side with different total lengths of DNA sequences (5-25 nt).

FIG. 8 depicts experimental data demonstrating the results from the testing of the ligation efficiency of two different tag sequences (initial sequences: GACTATC and GTGAGTT) with different GC/AT content.

FIG. 9 depicts experimental data demonstrating the results from the testing of the ligation efficiency of two different tag sequences (GACTATC and GTGAGTT) with different levels of phosphorylation.

FIG. 10 depicts experimental data demonstrating the results from the testing of the ligation efficiency of two different tag sequences (GACTATC and GTGAGTT) with asymmetrical tag overhangs.

FIGS. 11A-11D depict methods to evaluate qualitatively and quantitatively DNA durability. FIG. 11A depicts gel migration or DNA ligation followed by gel migration. Shown is the DNA ligation result (<50 bp) loaded on an acrylamide gel. FIG. 11B depicts DNA digestion followed by gel migration. FIG. 11C depicts DNA amplification and gel migration. FIG. 11D depicts LC-MS. Shown is an LCMS analysis of a short DNA sequence demonstrating high precision and a perfect molecular weight match. Left: Total ion chromatogram (TIC); Right: Deconvolution mass spectra.

FIGS. 12A-12C depict qPCR quantification of DNA following chemical reactions and comparison with ligation and gel electrophoresis quantification. FIG. 12A depicts the fluorescent signal (delta Rn) obtained for 8 serial dilutions corresponding to a positive control (DNA not treated). The negative control (no DNA) is shown on the right of the panel. FIG. 12B depicts the equation determination after fitting the results of Dil2-8 from above (Log 10 scale). FIG. 12C depicts the results obtained for 5 representative samples with different conditions (as listed). The green bar indicates the positive control (DNA not treated). The values presented above each histogram bar represents the % to control obtained by plotting the average cT values onto the equation from B. The values presented under the acrylamide gel picture correspondent to the DNA ligation efficiency of two fragments after DNA treatment.

FIG. 13 depicts the results of 3 independent experiments demonstrating an LC-MS analysis following chemical treatment of DNA.

FIG. 14 depicts a flow chart diagram for DNA ligation, PCR amplification and DNA sequencing of DNA fragments that have been submitted to chemical treatments/different treatments to test their integrity.

FIG. 15A through FIG. 15D depict diagrams showing the method for LCMS—detection and relative quantification of the generated DNA species with molecular resolution.

FIG. 16A through FIG. 16C depict exemplary experimental results demonstrating the evaluation of DNA durability in different conditions (pH and buffer) at 3 different temperatures in function of time (15 minutes to 24 hours). FIG. 16A depicts an analysis of different buffer and pH conditions at 150° C. FIG. 16B depicts an analysis of different buffer and pH conditions at 120° C. FIG. 16C depicts an analysis of different buffer and pH conditions at 100° C.

FIG. 17A through FIG. 17C depict exemplary experimental results demonstrating the evaluation of the DNA durability of different tags (natural and non-natural DNA). FIG. 17A depicts the DNA durability based on GC content. FIG. 17B depicts the DNA durability of three sequences with 50% GC. FIG. 17C depicts the DNA durability of a non-natural DNA variant.

FIG. 18A through FIG. 18C depict exemplary experimental results demonstrating the impact of acidic conditions on 8 different nucleotide sequences. FIG. 18A depicts the impact of acidic condition based on GC content. FIG. 18B depicts the the impact of acidic conditions on three sequences with 50% GC. FIG. 18C depicts the the impact of acidic condition DNA containing a non-natural DNA variant.

FIG. 19 depicts a diagram of a method to enhance capabilities of existing DNA-encoded libraries post synthesis.

FIG. 20 depicts exemplary features that can be added to a pre-existing DEL library, and enzymes that can be used to modify a pre-existing DEL library.

FIG. 21 depicts exemplary experimental results of the separation of small DNA fragments on acrylamide gel.

FIG. 22 depicts exemplary DNA sequences used to confirm that any enzyme (as long as they do not cut the DNA label) can be used to modify a pre-existing DEL library. The examples correspond to the enzyme examples presented in FIG. 20.

FIG. 23 depicts examples of 2 fragments ligated to add two modifications (Biotin and DIG).

FIG. 24 depicts an example of a method for confirmation of 1 Post-modification.

FIG. 25 depicts exemplary experimental results of evaluating and quantifying the compatibility of non-natural DNA molecules for DEL. Red marks indicate modified nucleotides.

FIG. 26 depicts an exemplary method for modifying DNA to increase its solubility in non polar solvents (e.g. organic solvents).

FIG. 27 depicts exemplary schemes for the construction of a DNA blocker and the modification of the DNA blocker to become a DNA solubilizer.

FIG. 28 depicts an example of the generation of a library of molecules barcoded with reversible partial DNA labels with altered solubility.

FIG. 29A through FIG. 29C depict a description of the SELDA assay. FIG. 29A depicts a schematic representation of a DNA encoded molecule with its DNA label. The chemical compound (colored circles and yellow hexagon) is covalently linked by a chemical linker to a DNA sequence called DNA label. The DNA label is composed of a 5′ label fragment (necessary for PCR amplification and sequencing) attached to the chemical linker, one or more barcoding Tags (e.g. shown is a set of 3 tags for a 3 split-and-pool strategy: Tag #1, Tag #2, Tag #3 represented in red, blue and purple respectively), and a final terminal fragment (3′ label) that serves several purposes including PCR amplification and sequencing, and that could be used for post-synthesis modifications as described in FIG. 20. Tag annotations are shown with their respective colors and with an example in which 3 series of 20 tags were used. FIG. 29B depicts three schematic configurations of all components necessary for SELDA showing the positive control (left), the negative control (middle), and a tag (e.g., Tag #1) to be tested (right). FIG. 29C depicts representations of 3 series of DNA labels (series for Tag #1, Tag #2 and Tag #3) and their corresponding DNA junctions (e.g., a 3 nucleotide overhang). The top label (Tag #1 in red) corresponds to the label represented in FIG. 29B (right panel).

FIG. 30A through FIG. 30B depict analysis of signal in positive and negative controls. FIG. 30A depicts the stability of the luminescent signal emitted by the positive and negative controls over time (0 to 120 min). FIG. 30B depicts an analysis of different negative controls (biotin, DIG, biotin-DIG) in SELDA comparing to the total signal emitted by the anti-DIG/HRP antibody diluted a million times. All values are expressed in net Relative Light Unit (RLU).

FIG. 31A through FIG. 31C depict the chemiluminescence detection and SELDA calibration. FIG. 31A depicts the chemiluminescence signal obtained in presence of increasing concentrations of anti-DIG antibody. The antibody precipitation and detection limits are indicated with dotted lines and non-viable conditions are hatched on the graph. The maximum chemiluminescence signal obtained with an Envision reader is ˜140 million RLU. FIG. 31B and FIG. 31C depict the chemiluminescence signal obtained in function of the Tag concentration for different concentrations of the anti-DIG^{HRP} antibody (dilutions 5·10⁻³, 2.5·10⁻⁴, 10⁻⁵ in FIG. 31B, and dilutions 10⁻⁶, 10⁻⁷, 10⁻⁸ in FIG. 31C). The dashed line rectangle indicates the optimal Tag concentration range for SELDA.

FIG. 32A through FIG. 32F depict an evaluation using SELDA of various parameters on ligation efficiency for different Tag designs. For each Tag design evaluated two Tag versions were tested (Tag-A yellow and Tag-B purple bars). Positive and negative controls are shown with grey and black bars respectively. The red arrows on the schematics highlight the parameter tested and the differences. FIG. 32A depicts a schematic representation of four Tag versions with different overhang lengths (1, 2, 3 or 5 nt) and the graph corresponding to the ligation efficiency results. FIG. 32B depicts the ligation efficiency of 9 Tag versions with increasing numbers of DNA nucleotides (5 to 25 nt). FIG. 32C depicts the ligation efficiency in function of the GC/AT nucleotides content (0, 25, 50, 75 or 100% of GC). FIG. 32D depicts the tag ligation efficiency in function of 5′-phosphate content (100, 80, 60, 40, 20 or 0% of 5′-phosphate). FIG. 32E depicts the tag ligation efficiency in function of an asymmetrical overhang ligation (2 vs. 4 nucleotides). FIG. 32F depicts the ligation efficiency based on the presence of non-natural nucleotides.

FIG. 33A through FIG. 33F depict testing of 60 DNA tags in 96-well format corresponding to 3 series of Tags mimicking the construction of a 3 split-and-pool steps DEL library. FIG. 33A depicts results corresponding to the ligation efficiency of 20 Tag #1(FIG. 33A), 20 Tag #2 (FIG. 33C) and 20 Tag #3 (FIG. 33E) and their respective positive and negative controls. Ligation efficiencies are expressed in percentage of Relative Light Unit (RLU). Analysis of ligation products by agarose gel electrophoresis are presented for 3 Tag #1 (FIG. 33B), 3 Tag #2 (FIG. 33D) and 3 Tag #3 (FIG. 33F). The numbers indicated correspond to the tags results presented on the left of each series.

FIG. 34A through FIG. 34C depict the impact of Tag concentration for 3 series of Tags. Ligation efficiency in function of an increasing concentration of 3 Tag #1 (FIG. 34A), 3 Tag #2 (FIG. 34B) and 3 Tag #3 (FIG. 34C). All tag numbers are indicated and correspond to the Tags tested in FIG. 33. Ligation efficiency is expressed in percentage of Relative Light Unit (RLU).

FIG. 35 depicts the ligation efficiency results of 3 Tag #1 (top), 3 Tag #2 (middle) and 3 Tag #3 (bottom) in function of the ligation duration. Ligation efficiency expressed in percentage of Relative Light Unit (RLU).

FIG. 36 depicts the ligation efficiency results of 3 Tag #1 (top), 3 Tag #2 (middle) and 3 Tag #3 (bottom) in function of the ligation temperature. Ligation efficiency expressed in percentage of Relative Light Unit (RLU).

FIG. 37A through FIG. 37C depict the impact of long-term storage on DNA ligation efficiency. Comparison of the DNA ligation efficiencies performed with SELDA of 12 different DNA Tags before (plain colored bars, as shown in FIG. 33A, FIG. 33C and FIG. 33E) and after a 2-year period of storage at −80 C (hatched colored bars). Tag #1s corresponding to Series #1 (FIG. 37A) (1.3, 1.6, 1.11 and 1.20), Tag #2s corresponding to Series #2 (FIG. 37B) (2.6, 2.9, 2.14 and 2.20) and Tag #3s corresponding to Series #3 (FIG. 37C) (3.5, 3.9, 3.13 and 3.20). Ligation efficiencies are expressed in percentage of Relative Light Unit (RLU).

FIG. 38A through FIG. 38E depict the impact of various chemical conditions on DNA ligation efficiency. Various DNA Tags used previously were treated in conditions relevant for DEL library construction to extend the usefulness of SELDA to DNA durability testing. The ligation efficiencies presented correspond to the value obtained for each Tag after treatment normalized to their respective non-treated values (100% RLU). Ligation efficiencies are expressed in percentage of Relative Light Unit (RLU). The representative conditions used are as follows: sodium-phosphate buffer 250 mM pH 5.5 thermo-treated at 120° C. for 2 h (FIG. 38A), sodium-phosphate buffer 250 mM pH 6.5 thermo-treated at 120° C. for 2 h (FIG. 38B), sodium-phosphate buffer 250 mM pH 6.5 thermo-treated at 120° C. for 16 h (FIG. 38C), borate buffer 150 mM pH 8.5 thermo-treated at 120° C. for 16 h (FIG. 38D) and borate buffer 150 mM pH 8.5, thermo-treated at 150° C. for 3 h (FIG. 38E). Tag #1 (1.4, 1.12, 1.14 and 1.15), Tag #2 (2.8, 2.15, 2.16 and 2.18) and Tag #3 (3.11, 3.12, 3.15 and 3.16).

FIG. 39 depicts a schematic diagram demonstrating a method to effectively measure, optimize and calibrate small DNA fragment ligation efficiency only on 1 side.

FIG. 40A through FIG. 40E depict a presentation of the concept for modifying DNA hydrophilicity in a reversible way, in order to solubilize DNA in organic solvents. FIG. 40A depicts a classical DEL partial DNA fragment used for DEL tagging that will be altered reversibly. A double strand DNA sequence is shown as Xs (blue open rectangle); dark blue rectangle: one DNA tag1 coding for chemical block1. FIG. 40B depicts data demonstrating that the DNA from FIG. 40A is fused to a binary entity that is a hybrid between another DNA fragment (orange rectangle) and a chemical compound (orange oval). An example is shown where the chemical compound is polyethylene glycol (PEG). FIG. 40C depicts data demonstrating that the DNA fragment (DNA blocker) will block on one end the free end of the DEL partial DNA label, and on the other end the presence of a free functional group (e.g., NH₂) will allow chemical addition of compounds (e.g., PEG). The green rectangle indicates the presence of an enzymatic restriction site needed at the end of the process to remove the DNA blocker. FIG. 40D depicts data demonstrating an example of the chemical moiety added to the DNA blocker PEG-SCM with NHS group is shown. FIG. 40E depicts data demonstrating a schematic representation of the DNA ligation covalently linking the DEL partial DNA label and the DNA solubilizer. The resulting product is hypothesized to be soluble in organic solvents (organic soluble DNA or osDNA). Abbreviations: NHS: N-Hydroxysuccinimide; mPEG-SCM: methoxy PEG-succinimidyl carboxyl methyl ester. Color code: Light blue background indicates solubility in H₂O. Light yellow background indicates solubility in organic solvents.

FIG. 41A through FIG. 41E depict a presentation of the concept for modifying DNA hydrophilicity in a reversible way, in order to solubilize DNA in organic solvents (continued). FIG. 41A depicts a schematic representation of adding a chemical block (green hexagon) onto the DEL scaffold. FIG. 41B depicts data demonstrating that following the addition of the desire chemical block, the solubilizer can be removed by enzymatic digestion represented by the red line and be ready for the next step ligation which is adding the next DEL tag (e.g., tag2; red box). FIG. 41C depicts data demonstrating that deprotection of the DEL DNA using a regular restriction enzyme (type 1) implies that the enzymatic cut is overlapping with the recognition site (red line inside the green box). The right scheme indicates that 7 extra nucleotides will be left out fused to the DEL label each time a DNA blocker is removed using a type 1 restriction enzyme. FIG. 41D depicts data demonstrating that a less frequently used enzyme (type 2) will cut the DNA outside the recognition sequence (red line outside the green box) and will lead to only 1 extra nucleotide being added to the DEL label. FIG. 41E depicts data demonstrating that two example of type 2 enzymes are shown: BsaI cutting 1 and 5 nucleotides away from the recognition sequence (1 extra nucleotide remains after digestion) and BbsJ cutting 2 and 6 nucleotides away from the recognition sequence (2 extra nucleotides remain after digestion). Color code: Light blue background indicates solubility in H₂O. Light yellow background indicates solubility in organic solvents. Red line: enzymatic digestion.

FIG. 42A and FIG. 42B depict the molecular details of a linker bridging a DNA blocker to a PEG molecule. FIG. 42A depicts an orange open rectangle corresponding to orange rectangles presented in FIGS. 40 and 40. The linker bridges the DNA blocker (left) to the PEG molecule (right) is composed of 2 spacers and a free amine (NH2) group. The DNA sequence can be any complementary double strand sequence (Xs/Xs) and of any length. FIG. 42B depicts data demonstrating that different modified PEG molecules can be used (see FIG. 40) and of different sizes (e.g., MW 1,000 to 20,000, which translate into 20 to 450 PEG units). Four examples of mPEG-NHS are presented: PEG-SCM: PEG-succinimidyl carboxymethyl ester; PEG-SPA: PEG-succinimidyl propionate; PEG-SAS: PEG-succinamide succinimidyl ester; PEG-SVA: PEG-succinimidyl valerate.

FIG. 43A through FIG. 43C depict a test of DNA solubility using various solutions on a simple and small DNA fragment. FIG. 43A depicts data demonstrating that any DNA fragment when precipitated with ethanol (100%) is found after centrifugation in the pellet and the supernatant (ethanol) does not contain DNA. The DNA is not soluble in pure ethanol. The pellet of DNA can be resuspended back in H₂O (light blue background) but not in organic solvent (e.g., DMSO)(light yellow background). FIG. 43B depicts data demonstrating the chemical reaction used to add a PEG molecule onto a DNA modified DEL fragment (blue rectangle; as shown in FIG. 42A). FIG. 43C depicts data demonstrating that six different PEG molecules (MW 1,000; 2,000; 3,400; 5,000; 10,000 and 20,000) were added onto the DNA fragment, precipitated with ethanol 100% and the amount of DNA present in the supernatant was analyzed by LCMS. The spectra on the left correspond to UV spectra and on the right to deconvoluted total ion chromatograms. The DNA-PEG molecules were detected (see arrows) in all 6 cases in the ethanol supernatant with an increase proportional to the PEG molecule size; the largest PEG molecule (MW 20,000) led to the highest quantity of DNA solubilized in pure ethanol.

FIG. 44 depicts a representative LCMS spectrum obtained when analyzing DNA-PEG molecules. The spectrum presented correspond to a DNA-PEG fragment that harbors a PEG molecule of MW 20,000 analyzed by LCMS; the spectrum corresponds to deconvoluted total ion chromatogram. The spectrum is quite diffuse due to the nature of the PEG molecule. The commercially available PEG molecules are synthetized using an enzyme that cannot be controlled precisely, and therefore the PEG molecules have a MW corresponding to the one indicated +/−10%. The top of the symmetrical peak corresponds to the theoretical MW as indicated.

FIG. 45A through FIG. 45D depict a test of DNA solubility using an actual DEL DNA fragment. FIG. 45A depicts data demonstrating that the DEL DNA fragment (blue open rectangle and dark blue rectangle) is covalently ligated with a T4 DNA ligase to a DNA solubilizer (PEG-5,000) and the overall MW of the osDNA (MW ˜25,000) is confirmed in FIG. 45B by LCMS. FIG. 45C depicts the osDNA construct from FIG. 45A (PEG-5,000) is dissolved following ethanol precipitation in 6 different organic solvents of decreasing polarity (DMSO (black), DMF (red), DMA (green), 1,4-dioxane (blue), ACN (purple), DCM (brown)). The expected molecular weight is approximately 25,000. The open black oval indicates positive detection. The spectra on the left correspond to UV spectra and on the right to deconvoluted total ion chromatograms. FIG. 45D depicts the osDNA construct this time containing a PEG moiety of MW 20,000 is dissolved in 6 different organic solvents of decreasing polarity (DMSO (black), DMF (red), DMA (green), 1,4-dioxane (blue), ACN (purple), DCM (brown)). The expected molecular weight is approximately 34,000. The open black oval indicates positive detection. The spectra on the left correspond to UV spectra and on the right to deconvoluted total ion chromatograms. Abbreviations: DMSO: dimethyl sulfoxide; DMF: dimethyl-formamide; DMA: dimethylacetamide; ACN: acetonitrile; DCM: dichloromethane.

FIG. 46A and FIG. 46B depict data demonstrating an amidation reaction in organic solvent of an osDNA fragment. FIG. 46A depicts that the compound presented on the left (MW 536) is added onto the osDNA using an amidation reaction in organic solvent (e.g., DMSO). This reaction would not be possible in aqueous solution. A PEG molecule of MW 5,000 was enough to allow the reaction. FIG. 46B depicts results of the reaction and LCMS spectra analysis are presented before and after the reaction. The shift of approximately MW 500 (actual 536) is highlighted by the two doted lines (red before reaction and blue after reaction). Due to the osDNA size, the resolution does not allow to see precisely an increase of MW 536 but the shift is clear.

FIG. 47A through FIG. 47C depict data demonstrating the reversibility of osDNA and confirmation of added compound molecular weight. FIG. 47A depicts data demonstrating that following the addition of the desire chemical block, the PEG-DNA can be removed by enzymatic digestion represented by the red line. FIG. 47B depicts data demonstrating that a less frequently used enzyme (type 2) will cut the DNA outside the recognition sequence (red line outside the green box) and will lead to only 1 extra nucleotide being added to the DEL label (red arrow). FIG. 47C depicts the results of the reaction and LCMS spectra analysis are presented after digestion. The expected shift of 537 is highlighted by the two doted lines (blue: starting material; red: product).

FIG. 48A and FIG. 48B depict a schematic representation of a classical DEL headpiece molecule use as starting material to build a DEL. FIG. 48A depicts an eight-nucleotide double strand DNA with 3 nucleotides overhang is shown (left) and a molecular zoom of the nucleotide structure is presented on the right. The yellow circle indicates the possible loss of phosphate; the purple oval shape indicate the loss of a base; the blue oval shape indicate the loss of an internal base; the orange oval represents the loss of a nucleotide; The pink circle represents the loss of the entire nucleotide and the second phosphate (the small purple circle). All species of modified DNA can be actually observed. FIG. 48B depicts a color code showing all possible DNA species that can be detected by LCMS. Abbreviations and annotations: FL: intact full length DNA piece; —Phos: minus 1 phosphate group; —G: minus 1 guanine; —G-G: minus 2 guanines; —3G: minus 3 guanines; —A: minus 1 adenine; —A-A: minus 2 adenines; —T: minus 1 thymine; —C: minus 1 cytosine; No DNA: DNA not detected; —2^{nd} Phos: the first and the second phosphates are missing; —CG: a cytosine and guanine are missing; —GA: a guanine and an adenine are missing; —2 GA: 2 guanines and 1 adenine are missing; —GA Phos: 1 guanine, 1 adenine and 1 phosphate group are missing; —Base: the base only of a given nucleotide is missing; a small colored square: the species corresponding to the square color was just detected (not quantified); a number inside a small square: more than 1 was detected missing; a number x (1 to 8) in black in a large rectangle: x entire base(s) are missing; a number x (1 to 8) in red in a large rectangle: x entire nucleotide(s) are missing. MW: molecular weight; A, C, T, G: the four DNA nucleotides adenine, cytosine, thymine and guanine.

FIG. 49A through FIG. 49C depict specific examples of representative LCMS results obtained after different treatments. FIG. 49A depicts UV spectra with various DNA species peaks highlighted. FIG. 49B depicts spectra corresponding to deconvoluted total ion chromatograms of the results from Peaks corresponding to the loss of the different components are highlighted with the color codes presented in FIG. 48A. FIG. 49C depicts a schematic representation of the results using the symbolism from FIG. 48A. Abbreviations and annotations: FL: intact full length DNA piece MW 6,517; —Phos: minus 1 phosphate group; —G: minus 1 guanine; —GP: 1 guanine and 1 phosphate group are missing; —Base: the base only of a given nucleotide is missing; MW: molecular weight. If not specified, —A, C, T or G indicates the loss a nucleotide (otherwise—Base indicates that only the base was lost).

FIG. 50A through FIG. 50C depict the evaluation of quality following diverse treatments. Unless specified, the DNA was analyzed without dilution. The dilution factors indicated correspond to loading dilutions. The time indicated is expressed in hour(s) FIG. 50A depicts representative results obtained by agarose gel electrophoresis of DNA exposed to different treatments. Top left: 250 mM pH 8.0 sodium phosphate; Top right and bottom left: 250 mM pH 5.5 sodium phosphate; bottom right: 250 mM pH 6.5 sodium phosphate. FIG. 50B depicts an example of results obtained when comparing three different treatments. Each rectangle represents a time point (30 minutes to 24 hours) and the height of the vertical black bars is proportional to the total amount of DNA detected. Top results: 250 mM pH 8.0 sodium phosphate; middle results: 250 mM pH 5.5 sodium phosphate; bottom results: 250 mM pH 6.5 sodium phosphate. FIG. 48C depicts an example of results obtained when comparing three different repeats for 1 single treatment (sodium phosphate buffer, pH6.5, 120° C.). Each rectangle represents a time point (30 minutes to 24 hours) and the height of the vertical black bars is proportional to the total amount of DNA detected.

FIG. 51A through FIG. 51C depict studies evaluating the impact of 3 extreme temperatures, 4 different buffers and 11 pH conditions on a DEL DNA headpiece. Results obtained with different treatments are organized based on the temperature: (FIG. 51A) at 100° C., (FIG. 51B) at 120° C. and (FIG. 51C) at 150° C. Each rectangle represents a time point (30 minutes to 24 hours) and the height of the vertical black bars is proportional to the total amount of DNA detected. The buffers used are indicated on the left together with the pH for each of these conditions. White empty rectangles indicate that the condition has not been tested based on the results of previous time points.

FIG. 52A through FIG. 52E depict studies evaluating the impact of 5 temperatures and 2 different buffers in basic conditions on a DEL DNA headpiece. Results obtained with different treatments are organized based on the temperature: (FIG. 52A) at 100° C., (FIG. 52B) at 110° C., (FIG. 52C) at 120° C., (FIG. 52D) at 130° C. and (FIG. 52E) at 140° C. For each temperature two basic conditions were evaluated (PH 9.5 (top) and 11.0 (bottom)). Treatment times vary from 15 minutes to 125 hours as indicated for each rectangle.

FIG. 53A through FIG. 53F depict studies evaluating the impact of the DNA composition in 5 conditions and in acidic conditions on a DEL DNA headpiece. FIG. 53A: Five DNA sequences with a percentage of GC bases varying from 0% to 100% were used. The GC % is indicated on the left and the treatment conditions (buffer, temperature, incubation time) are listed above. FIG. 53B: The DNA used in FIG. 53A were tested in very acidic conditions (pH 4.5) in sodium acetate buffer (100° C., 30 minutes) and represented with a long color gradient as numerous species were found or detected. FIG. 53C: Three DNA sequences with 50% GC bases with 1 (middle row) or 2 (lower raw) nucleotides difference were tested. The nucleotide difference is indicated on the left and the treatment conditions (buffer, temperature, incubation time) are listed above. +1 nt: 1 nucleotide is different; switch 2 nt: 2 nucleotides were switched. FIG. 53D: The DNA used in FIG. 53C were tested in very acidic conditions (pH 4.5) in sodium acetate buffer (100° C., 30 minutes) and represented with a long color gradient as numerous species were found or detected. FIG. 53E depicts an example of non-natural DNA was tested in 5 conditions as shown for FIG. 53A and FIG. 53C. The schematic indicates that all nucleotides of 1 DNA strand were replaced with Inosine nucleotides. FIG. 53F: The progressive removal of every single inosine nucleotide was observed as visualized by the brown gradient.

FIG. 54A through FIG. 54N depict an evaluation of the impact on DNA integrity of a number of chemical reagents and conditions commonly used in organic chemistry. Among other parameters, 8 catalysts, 2 solvents and 2 bases never used in the DEL field were tested, as well as scavenger molecules, other ligands, reagents and conditions. FIG. 54A: UV pattern in Condition 1 after treating with C8. FIG. 54B: UV pattern and deconvoluted mass spectrum (i) C3 in Condition 1; (ii) C3 in Condition 3; (iii) C4 in Condition 4; (iv) C7 in Condition 4; (v) C8 in Condition 4; (vi) C9 in Condition 3. FIG. 54C: UV pattern in Condition 1 after treating with C1. FIG. 54D: UV pattern after treating 10 hours with C1 in various conditions (i) Condition 2; (ii) Condition 3; (iii) Condition 4. FIG. 54E: Before and after Scavengers treatment UV pattern after treating with C10 in Condition 2. FIG. 54F: Before and after Scavenger 1 treatment UV pattern after treating with C1 in Condition 2. FIG. 54G: Before and after Scavenger 1 treatment UV pattern after treating with C2 in Condition 4. FIG. 54H through FIG. 54J: UV peaks after treating with ligands in condition 6. FIG. 54K: UV peaks after treating with Hydrogen peroxide (H₂02) at different pHs. FIG. 54L: UV peaks after treating with Azobisisobutyronitrile (AIBN) in condition 5. FIG. 54M: UV peaks after treating with Azobisisobutyronitrile (AIBN) in condition 6. FIG. 54N: UV peaks after treating with Cyclodextrin in condition 6. Condition 1: 250 mM pH 6.5 sodium phosphate/DMSO (3:1). Condition 2: 150 mM pH 6.5 sodium borate/DMSO (3:1). Condition 3: H₂O/DMSO (3:1). Condition 4: DMSO/150 mM pH 9.5 sodium borate/3:1. Condition 5: 250 mM pH 6.5 sodium phosphate/DMA (4:1). Condition 6: 150 mM pH 9.5 sodium borate/DMA (4:1). Catalyst (Cx): C1: Cp*RuCl(PPh₃)₂ Ru(II); C2: [Ir(cod)Cl]₂ lr(I); C3: Cp₂Ni; C4: CoBr₂; C7: AuI; C8: AgOTf; C9: CuI; C10: sSPhos Pd G2. Ligands (Lx): L1: Xanthphos; L2: L3: tBuBrettPhos; L6: DPEphos. Scavenger 1: Sodium diethyldithiocarbamate (NaDEDTC); Scavenger 2: 2-Mercaptoethanol (BME). T: time in hours. Abbreviations: Ag: Bor: Ru: DMSO: Scav.: scavenger. Numbers in red: pH. Text in blue: specific treatment, scavenger or ligand.

### DETAILED DESCRIPTION

The present invention relates to methods for designing tags for efficient ligation to DNA libraries, compositions comprising the optimized tags sets and methods of use thereof for tagging DNA libraries. In one embodiment, the methods of the invention have been developed to reduce or prevent the inclusion of tags with low ligation efficiency in a set of DNA labeling tag or barcode sequences, increasing the quality of the data, normalize the ligation efficiency across all tags within a tags set and improve the counts comparison for sequence data and increase the signal to noise ratio in data generated from a DNA encoded library (e.g., DNA sequencing data). The invention is based, in part, on the development of enzyme-linked DNA-ligation assays, including a sandwich enzyme-linked DNA-ligation assay (SELDA), and the use thereof to measure and calibrate DNA tag ligation efficiency.

In one embodiment, the enzyme-linked DNA-ligation assay comprises (a) a substrate for attaching a capture oligonucleotide; (b) a capture oligonucleotide comprising a moiety for attaching the capture oligonucleotide to the substrate; (c) a test nucleic acid molecule comprising a moiety for recognition by a detectable moiety and (d) a detectable moiety for detection of the ligation between the capture oligonucleotide and the test nucleic acid molecule (e.g., oligonucleotide). In one embodiment, the DNA ligation assay of the invention comprises a capture DNA oligonucleotide that is covalently immobilized to a surface (e.g., a well) and a test nucleic acid molecule that harbors a moiety for direct or indirect colorimetric, chemiluminescent or fluorescent detection. In some embodiments, the method comprises contacting the capture oligonucleotides with the test nucleic acid molecule and detecting a colorimetric, chemiluminescent or fluorescent signal upon ligation of the capture oligonucleotide to the test nucleic acid molecule. The signal generated when all the pieces are assembled successfully is proportional to the ligation efficiency of the capture oligonucleotide to the test nucleic acid molecule.

In one embodiment, the invention relates to a sandwich enzyme-linked DNA-ligation assay (SELDA). In one embodiment, SELDA comprises (a) a substrate for attaching a first accessory oligonucleotide; (b) a first accessory oligonucleotide comprising a moiety for attaching the first oligonucleotide to the substrate; (c) a second accessory oligonucleotide comprising a moiety for recognition by a detectable moiety and (d) a detectable moiety for detection of the ligation between the first accessory oligonucleotide, an intermediary test nucleic acid molecule (e.g., oligonucleotide) and the second accessory nucleic acid molecule (e.g., oligonucleotide). In one embodiment, the SELDA method of the invention comprises the use of a sandwich assay comprising a first accessory DNA oligonucleotide that is covalently immobilized to a surface (e.g., a well) and a second enzyme-linked accessory DNA oligonucleotide that harbors a moiety for recognition by an antibody comprising a detectable moiety for colorimetric, chemiluminescent or fluorescent detection. In some embodiments, the method comprises contacting the first and second accessory oligonucleotides with the intermediary test oligonucleotide and detecting a colorimetric, chemiluminescent or fluorescent signal upon ligation of the intermediary test oligonucleotide to the first and second accessory fragments. The signal generated when all the pieces are assembled successfully is proportional to the ligation efficiency of the intermediary test oligonucleotide, on both ends at once.

In one embodiment, the intermediary test oligonucleotide is a oligonucleotide molecule for tagging a nucleic acid molecule. In one embodiment, the invention provides methods for generating optimized sets of tag oligonucleotides determined by the DNA ligation assay of the invention to have comparable ligation efficiencies. In one embodiment, the invention provides optimized sets of tag oligonucleotides determined by the DNA ligation assay of the invention to have comparable ligation efficiencies. In one embodiment, the invention provides methods of using the optimized tag oligonucleotide sets to construct and tag a DNA encoded library (DEL).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

“About” as used herein when referring to a measurable value such as an amount, a temporal duration, and the like, is meant to encompass variations of 20% or ±10%, more preferably +5%, even more preferably +1%, and still more preferably ±0.1% from the specified value, as such variations are appropriate to perform the disclosed methods.

The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, an “adaptor” of the present invention means a piece of nucleic acid that is added to a nucleic acid of interest, e.g., the polynucleotide. Two adaptors of the present invention are preferably ligated to the ends of a DNA fragment cross-linked to a polypeptide of interest, with one adaptor on each end of the fragment. Adaptors of the present invention can comprise a primer binding sequence, a random nucleotide sequence, a barcode, or any combination thereof.

An affinity label, as the term us used herein, refers to a moiety that specifically binds another moiety and can be used to isolate or purify the affinity label, and compositions to which it is bound, from a complex mixture. One example of such an affinity label is a member of a specific binding pair (e.g., biotin:avidin, antibody:antigen). The use of affinity labels such as digoxigenin, dinitrophenol or fluorescein, as well as antigenic peptide ‘tags’ such as polyhistidine, FLAG, HA and Myc tags, is envisioned.

“Amplification,” as used herein, refers to any in vitro process for increasing the number of copies of a nucleotide sequence or sequences, i.e., creating an amplification product which may include, by way of example additional target molecules, or target-like molecules or molecules complementary to the target molecule, which molecules are created by virtue of the presence of the target molecule in the sample. These amplification processes include but are not limited to polymerase chain reaction (PCR), multiplex PCR, Rolling Circle PCR, ligase chain reaction (LCR) and the like, in a situation where the target is a nucleic acid, an amplification product can be made enzymatically with DNA or RNA polymerases or transcriptases. Nucleic acid amplification results in the incorporation of nucleotides into DNA or RNA. As used herein, one amplification reaction may consist of many rounds of DNA replication. PCR is an example of a suitable method for DNA amplification. For example, one PCR reaction may consist of 2-40 “cycles” of denaturation and replication.

“Amplification products,” “amplified products” “PCR products” or “amplicons” comprise copies of the target sequence and are generated by hybridization and extension of an amplification primer. This term refers to both single stranded and double stranded amplification primer extension products which contain a copy of the original target sequence, including intermediates of the amplification reaction.

As used herein, an “antibody” encompasses naturally occurring immunoglobulins, fragments thereof, as well as non-naturally occurring immunoglobulins, including, for example, single chain antibodies, chimeric antibodies (e.g., humanized murine antibodies), heteroconjugate antibodies (e.g., bispecific antibodies). Fragments of antibodies include those that bind antigen, (e.g., Fab′, F(ab′)2, Fab, Fv, and rlgG). See, e.g., Pierce Catalog and Handbook, 1994-1995 (Pierce Chemical Co., Rockford, III.); Kuby, J., Immunology, 3rd Ed., W.H. Freeman & Co., New York (1998). The term “antibody” further includes both polyclonal and monoclonal antibodies.

“Appropriate hybridization conditions” as used herein may mean conditions under which a first nucleic acid sequence (e.g., primer, etc.) will hybridize to a second nucleic acid sequence (e.g., target, etc.), such as, for example, in a complex mixture of nucleic acids. Appropriate hybridization conditions are sequence-dependent and will be different in different circumstances. In one embodiment, an appropriate hybridization conditions may be selective or specific wherein a condition is selected to be about 5-10° C. lower than the thermal melting point (Tm) for the specific sequence at a defined ionic strength and pH. In one embodiment, an appropriate hybridization condition encompasses hybridization that occurs over a range of temperatures from more to less stringent. In one embodiment, a hybridization range may encompass hybridization that occurs from 98° C. to 10° C. According to the invention, such a hybridization range may be used to allow hybridization of the primers of the invention to target sequences with reduced specificity, for the purposes of amplifying a broad range of nucleic acid molecules with a single set of primers.

A “barcode”, as used herein, refers to a nucleotide sequence that serves as a means of identification for sequenced polynucleotides of the present invention. Barcodes of the present invention may comprise at least 4, 5, 6, 7, 8, 9, 10, 11, 12, 13, 14, 15, 16, 17, 18, 19, 20 or more bases in length.

As used herein, “binding” means an association interaction between two molecules, via covalent or non-covalent interactions including, but not limited to, hydrogen bonding, hydrophobic interactions, van der Waals interactions, and electrostatic interactions. Binding may be sequence specific or non-sequence specific.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

As used herein, “dNTPs” refers to a mixture of different deoxyribonucleotide triphosphates: deoxyadenosine triphosphate (dATP), deoxycytidine triphosphate (dCTP), deoxyguanosine triphosphate (dGTP) and deoxythymidine triphosphate (dTTP).

DNA “durability” as used herein refers to the general overall molecular structure of the DNA molecule, in contrast to the term DNA “solubility” which as used herein refers to the ability of a double stranded helix to be denatured to become single strand.

“Intact” DNA as used herein refers to a DNA molecule in which the molecular structure remains unmodified.

“Fragment” as applied to a nucleic acid, refers to a subsequence of a larger nucleic acid. A “fragment” of a nucleic acid can be at least about 4 nucleotides in length; for example, at least about 4 nucleotides to about 25 nucleotides, at least about 4 nucleotides to about 50 nucleotides; at least about 4 to about 100 nucleotides, at least about 4 to about 500 nucleotides, at least about 4 to about 1000 nucleotides, at least about 4 nucleotides to about 1500 nucleotides; or about 4 nucleotides to about 2500 nucleotides (and any integer value in between).

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. When comparing DNA and RNA, thymine (T) and uracil (U) may be considered equivalent. Identity may be performed manually or by using a computer sequence algorithm such as BLAST or BLAST 2.0.

“Nucleic acid” or “oligonucleotide” or “polynucleotide” or “nucleic acid fragment” as used herein may mean at least two nucleotides covalently linked together. The depiction of a single strand also defines the sequence of the complementary strand, or the sequence of a molecule that hybridizes to at least a portion of the single strand sequence. Thus, a nucleic acid also encompasses the complementary strand of a depicted single strand as well as probes, primers or oligonucleotide sequences having complementarity to at least a portion of the strand. Many variants of a nucleic acid may be used for the same purpose as a given nucleic acid. Thus, a nucleic acid also encompasses substantially identical nucleic acids and complements thereof. A single strand provides a probe that may hybridize to a target sequence. Thus, a nucleic acid also encompasses a probe that hybridizes under appropriate hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. As used herein, the term nucleic acids includes both natural and non-natural nucleic acids. Non-natural nucleic acids include, but are not limited to, 2′F, 2′-fluoro; 2′OMe, 2′-O-methyl; LNA, locked nucleic acid; FANA, 2′-fluoro arabinose nucleic acid; HNA, hexitol nucleic acid; 2′MOE, 2′-O-methoxyethyl; ribuloNA, (1′-3′)-β-L-ribulo nucleic acid; TNA, α-L-threose nucleic acid; tPhoNA, 3′-2′ phosphonomethyl-threosyl nucleic acid; dXNA, 2′-deoxyxylonucleic acid; PS, phosphorothioate; phNA, alkyl phosphonate nucleic acid; and PNA, peptide nucleic acid.

As used herein, a “polypeptide of interest” may be any polypeptide for which said polypeptide's genomic binding regions are sought. It is envisioned that a polypeptide of the present invention may include full length proteins and protein fragments. While the methods of the present invention may be utilized not only to determine at least one region of a genome at which a polypeptide of interest binds, they may also be utilized to determine if a polypeptide binds to a genome at all. The polypeptide of interest may selected from the group consisting of a transcription factor, a polymerase, a nuclease, and a histone.

“Primer” as used herein refers to a single-stranded oligonucleotide or a single-stranded polynucleotide that is extended on its 3′ end by covalent addition of nucleotide monomers during amplification. Nucleic acid amplification often is based on nucleic acid synthesis by a nucleic acid polymerase. Many such polymerases require the presence of a primer that can be extended to initiate such nucleic acid synthesis.

As used herein, “sample” or “test sample,” may refer to any source used to obtain nucleic acids for examination using the compositions and methods of the invention. A test sample is typically anything suspected of containing a target sequence.

Any DNA sample may be used in practicing the present invention, including without limitation eukaryotic, prokaryotic, viral DNA, non-natural DNA, cDNA, and recombinant DNA molecules.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of about 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under appropriate hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

As used herein, a “substrate” is a solid platform or surface to which antibodies or nucleic acid molecules used in the assay system are bound.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention provides assays for improving DEL library construction including assays to identify nucleic acid molecules having high or similar ligation efficiencies for use in efficiently tagging DEL libraries, assays to identify molecules that increase the solubility of DEL in non-aqueous solvents as well as assays to identify modifications or solutions that increase the durability of DEL. The invention includes the use of the conditions and molecules identified according to the assay systems, alone or in any combination, to generate a DEL.

### Enzyme-Linked DNA-Ligation Assays

The invention is based, in part, on systems and methods for identifying nucleic acid molecules having high or similar ligation efficiencies as well as methods for the use of the nucleic acid molecules having high or similar ligation efficiencies for tagging DNA encoded libraries (DELs).

In some embodiments, the invention relates to an enzyme-linked DNA-ligation assay that can be used to determine ligation efficiency of one or more target oligonucleotide for inclusion in a set of tag oligonucleotides. In one embodiment, the assay is used to identify a set of oligonucleotide tags with similar or comparable ligation efficiencies that can be used for tagging a nucleic acid molecule library. In some embodiments, the nucleic acid molecule library is a DNA encoded library (DEL).

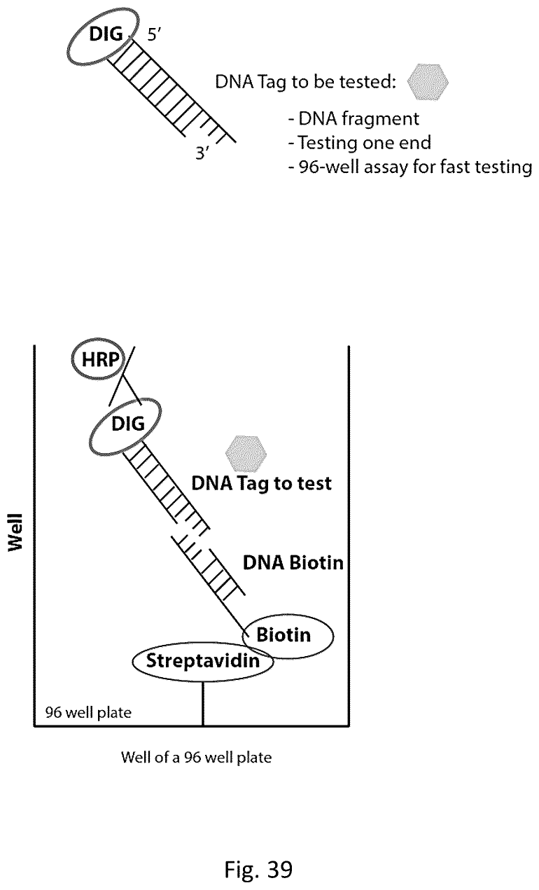

In one embodiment, the DNA-ligation assay comprises (a) a substrate for attaching a capture oligonucleotide; (b) a capture oligonucleotide comprising a moiety for attaching the capture oligonucleotide to the substrate; (c) a test nucleic acid molecule comprising a moiety for direct or indirect detection and (d) a detectable moiety for detection of the ligation between the capture oligonucleotide and the test nucleic acid molecule. In one embodiment, the DNA-ligation assay of the invention comprises the use of capture DNA oligonucleotide that is covalently immobilized to a surface (e.g., a well) and one or more test nucleic acid molecule(s) that harbors a moiety for recognition by a detectable moiety for colorimetric, chemiluminescent or fluorescent detection. In some embodiments, the method comprises contacting the capture oligonucleotide with one or more test nucleic acid molecule(s) and detecting a colorimetric, chemiluminescent or fluorescent signal upon ligation of the one or more test nucleic acid molecule(s) to the capture oligonucleotide. The signal generated when all the pieces are assembled successfully is proportional to the ligation efficiency of the test nucleic acid molecule to the capture oligonucleotide. A schematic diagram of one embodiment of the DNA-ligation assay is provided in FIG. 39.

In some embodiments, the invention relates to a sandwich enzyme-linked DNA-ligation assay (SELDA) that can be used to determine ligation efficiency of one or more target oligonucleotide for inclusion in a set of tag oligonucleotides. In one embodiment, the assay is used to identify a set of oligonucleotide tags with similar or comparable ligation efficiencies that can be used for tagging a nucleic acid molecule library. In some embodiments, the nucleic acid molecule library is a DNA encoded library (DEL).

In one embodiment, SELDA comprises (a) a substrate for attaching a first accessory oligonucleotide; (b) a first accessory oligonucleotide comprising a moiety for attaching the first oligonucleotide to the substrate; (c) a second accessory oligonucleotide comprising a moiety for recognition by a detectable moiety (i.e., antibody recognition moiety) and (d) a detectable moiety for detection of the ligation between the first accessory oligonucleotide, an intermediary test oligonucleotide and the second accessory oligonucleotide. In one embodiment, the SELDA method of the invention comprises the use of a sandwich assay comprising a first accessory DNA oligonucleotide that is covalently immobilized to a surface (e.g., a well) and a second enzyme-linked accessory DNA oligonucleotide that harbors a moiety for recognition by an antibody comprising a detectable moiety for colorimetric, chemiluminescent or fluorescent detection. In some embodiments, the method comprises contacting the first and second accessory oligonucleotides with the intermediary test oligonucleotide and detecting a colorimetric, chemiluminescent or fluorescent signal upon ligation of the intermediary test oligonucleotide to the first and second accessory fragments. The signal generated when all the pieces are assembled successfully is proportional to the ligation efficiency of the intermediary test oligonucleotide, on both ends at once.

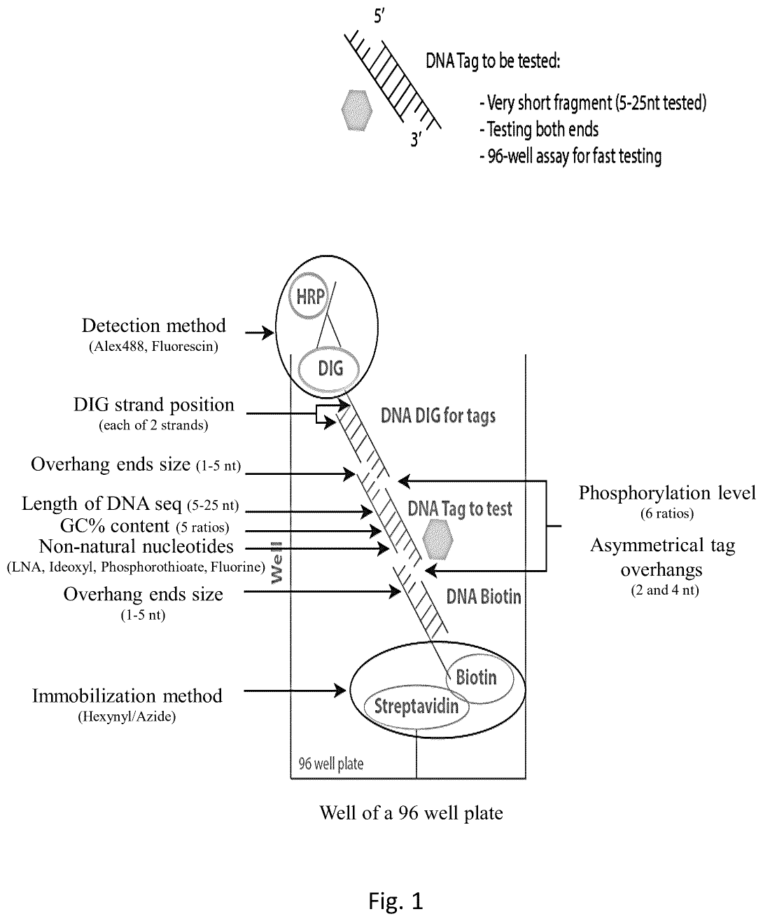

In some embodiments, SELDA involves a) contacting an intermediary test oligonucleotide molecule with a first accessory oligonucleotide conjugated to a surface and a second accessory oligonucleotide conjugated to moiety for direct or indirect detection, b) ligating the intermediary test oligonucleotide molecule to the first accessory oligonucleotide and the second accessory oligonucleotide, c) removing any unligated oligonucleotides by washing, d) contacting the ligated complexes with a detectable molecule for colorimetric, chemiluminescent or fluorescent detection, and e) determining the ligation efficiency of the oligonucleotide tag molecule by detecting the colorimetric, luminescencent or fluorescencent readout of the detectable molecule. A schematic diagram of SELDA is provided in FIG. 1.

In one embodiment, the first accessory oligonucleotide is conjugated to biotin and is attached to a streptavidin coated surface through a biotin-streptavidin interaction. However, the assay system is not limited to the use of a biotin-streptavidin interaction for attaching the first accessory oligonucleotide to a surface, but can use any method for linking an oligonucleotide to a surface including linking through the use of an antibody interaction or magnetic beads.

In one embodiment, the second accessory oligonucleotide is conjugated to digoxigenin (DIG) and subsequent detection is performed using high affinity anti-DIG antibodies, coupled either to alkaline phosphatase (AP), horseradish peroxidase (HRP), fluorescein or rhodamine for colorimetric, and chemiluminescent or fluorescent detection. However, the assay system is not limited to the use of a DIG:anti-DIG interaction as any method for detection of a ligated or bound oligonucleotide can be used for labeling and subsequent detection in a DNA ligation assay of the invention. Exemplary detection methods that can be used for labeling and subsequent detection include, but are not limited to, fluorophores, quantum dots, isotopes for radioactivity detection. Exemplary fluorophores that can be used include, but are not limited to, FITC, Alexa fluor 488 or 561, Cy5, Texas red, and rhodamine. In some embodiments, the second accessory oligonucleotide harbors a DNA sequence that could be recognized by an antibody, or used in a proximity ligation assay (PLA).

In one embodiment, a DNA ligation assay of the invention allows the identification of oligonucleotide molecules or sets of oligonucleotides with a desired ligation efficiency. This technique is generally applicable; a DNA ligation assay of the invention is capable of detecting ligation efficiency of any oligonucleotide molecule of interest. Exemplary oligonucleotides that can be evaluated for ligation efficiency using a DNA ligation assay of the invention include, but are not limited to, nucleic acid fragments, DNA fragments, non-natural DNA oligonucleotides, tagging oligonucleotides, barcode oligonucleotides, spacer oligonucleotides, oligonucleotides comprising restriction enzyme recognition sites, oligonucleotides comprising or encoding sequences for antibody recognition, a nucleic acid molecule conjugated to a molecule for binding or purification including, but not limited to, a nucleic acid-biotin conjugate, a nucleic acid-magnetic bead conjugate, a nucleic acid-antibody conjugate, or a nucleic acid-nanobody conjugate, a nucleic acid molecule conjugated to a molecule for visulaization including, but not limited to, a nucleic acid-fluorophor conjugate, and a nucleic acid-quantum dot conjugate, a nucleic acid molecule conjugated to a molecule for cell permeabilization including, but not limited to, a nucleic acid-cholesterol conjugate, a nucleic acid-polyArg conjugate, and a nucleic acid-TatSeq conjugate, or a nucleic acid molecule conjugated to a molecule for proximal PCR or rolling circle amplification (RCA).

In one embodiment, the oligonucleotide/nucleic acid molecule is for use as a tag. The tag oligonucleotide/nucleic acid molecule can be used to tag a nucleic acid library, including, but not limited to, a library of nucleic acid molecules. In one embodiment, the library is a DEL library.

In one embodiment a DNA ligation assay of the invention includes detection of the ligation efficiency of a test oligonucleotide or test nucleic acid molecule to at least one of a capture oligonucleotide, an accessory oligonucleotide, or a combination of two accessory oligonucleotides. In one embodiment, a DNA ligation assay of the invention comprises one or more wash steps to remove unbound oligonucleotides or accessory molecules. In one embodiment, buffer and wash conditions are of sufficient stringency (e.g., low sodium wash solutions at increased temperature) that unligated interactions of the target oligonucleotide and one or more accessory oligonucleotide are disrupted.

### Oligonucleotide Tags

In one embodiment, the target oligonucleotide tag molecule (e.g., a test nucleic acid molecule or intermediate test oligonucleotide) comprises a 5′ overhang, a 3′ overhang, or both a 5′ overhang and a 3′ overhang to promote ligation of the target oligonucleotide tag molecule to at least one of the capture, first and second accessory oligonucleotide molecules. In various embodiments, the overhang at the 5′ end of the target oligonucleotide tag molecule comprises at least 1, at least 2, at least 3, at least 4, at least 5 or more than 5 nucleotides. In one embodiment, the overhang at the 5′ end of the target oligonucleotide tag molecule comprises 3 nucleotides. In various embodiments, the overhang at the 3′ end of the target oligonucleotide tag molecule comprises at least 1, at least 2, at least 3, at least 4, at least 5 or more than 5 nucleotides. In one embodiment, the overhang at the 3′ end of the target oligonucleotide tag molecule comprises 3 nucleotides. In one embodiment, the target oligonucleotide tag molecule comprises an overhang at the 5′ end of the target oligonucleotide tag molecule comprising 3 nucleotides and an overhang at the 3′ end of the target oligonucleotide tag molecule comprising 3 nucleotides. In one embodiment, the target oligonucleotide tag molecule comprises an overhang at the 5′ end of the target oligonucleotide tag molecule comprising 2 nucleotides and an overhang at the 3′ end of the target oligonucleotide tag molecule comprising 4 nucleotides.

In one embodiment, the nucleotides in the overhang are pyrimidine nucleotides to promote stability and durability of the DEL.

In one embodiment, the total length of the target oligonucleotide tag molecule comprises at least 3, 4, 5, 6, 7, 8, 9, 10, 11, 12, 13, 14, 15, 16, 17, 18, 19, 20, 21, 22, 23, 24, 25, or more than 25 nucleotides. In one embodiment, total length of the target oligonucleotide tag molecule is 5-10 nucleotides. Therefore, in one embodiment, the target oligonucleotide tag molecule comprises from 5′ to 3′: a 3 nucleotide 5′ single stranded overhang, a 1-4 nucleotide double stranded region, and a 3 nucleotide 3′ single stranded overhang. In one embodiment, the target oligonucleotide tag molecule comprises from 5′ to 3′: a 2 nucleotide 5′ single stranded overhang, a 1-4 nucleotide double stranded region sequence, and a 4 nucleotide 3′ single stranded overhang.

In one embodiment, the oligonucleotide tag molecule comprises a modified backbone, a modified sugar, or a modified nucleobase. In one embodiment, the oligonucleotide tag molecule comprises at least 2, 3, 4, 5, 6 or 7 modified nucleobases or modified nucleotides. Modifications of nucleotides that can be included in the oligonucleotide tag molecule include, but are not limited to, 2′F, 2′-fluoro; 2′OMe, 2′-O-methyl; LNA, locked nucleic acid; FANA, 2′-fluoro arabinose nucleic acid; HNA, hexitol nucleic acid; 2′MOE, 2′-O-methoxyethyl; ribuloNA, (1′-3′)-β-L-ribulo nucleic acid; TNA, α-L-threose nucleic acid; tPhoNA, 3′-2′ phosphonomethyl-threosyl nucleic acid; dXNA, 2′-deoxyxylonucleic acid; PS, phosphorothioate; phNA, alkyl phosphonate nucleic acid; and PNA, peptide nucleic acid.

In one embodiment, the oligonucleotide tag molecule comprises at least one modified nucleotide. In one embodiment, the oligonucleotide tag molecule comprises at least 2, 3, 4, 5, 6, 7, 8, 9, 10 or more than 10 modified nucleotide. In one embodiment, the oligonucleotide tag molecule comprises at least one LNA. In one embodiment, the oligonucleotide tag molecule comprises at least 2, 3, 4, 5, 6, 7, 8, 9, 10 or more than 10 LNA.

### DNA Solubilizers

The invention is based, in part, on the development of molecules having the ability to increase solubility of DNA molecules in non-aqueous solutions or solvents, herein referred to as DNA solubilizers, as well as methods for the use of the DNA solubilizer molecules for generating DELs having increased or altered solubility.

The DNA solubilizer molecules allow for a DNA molecule to be dissolved in various organic solvents, and allows for chemical reactions that were not possible in aqueous solutions in the presence of DNA.

In some embodiments, the DNA solubilizer molecule comprises a molecule for modulating the solubility of DNA. Exemplary molecules for modulating the solubility of DNA include, but are not limited to, polyethylene glycol (PEG), methoxy PEG-succinimidyl carboxyl methyl ester, methoxy PEG-succinimidyl carboxyl methyl ester with a N-Hydroxysuccinimide group. In some embodiments, the molecule for modulating the solubility of DNA comprises a molecular weight between 500 and 50,000. In some embodiments, the molecule for modulating the solubility of DNA comprises a molecular weight of 1,000; 2,000; 3,400; 5,000; 10,000; 20,000 or more than 20,000.

In one embodiment, the DNA solubilizer molecule comprises a DNA blocker molecule covalently linked to the molecule for modulating the solubility of DNA. In one embodiment, the DNA blocker molecule blocks the ligatable end of the DEL DNA molecule from further ligation while undergoing a chemical reaction in an organic solvent. In one embodiment, the DNA blocker molecule comprises a restriction enzyme cleavage site that can be cleaved to remove the DNA solubilizer and restore the ligation capacity of the DEL DNA molecule.

In one embodiment, the invention provides an assay system to identify DNA solubilizer molecules that can alter the solubility of DNA molecules and DEL. In some embodiments, the DNA solubility assay includes the steps of: ligating a DEL DNA molecule, or a DNA molecule to be ligated to a DEL, to a DNA solubilizer molecule comprising a DNA blocker linked to a compound to be tested for its solubilizing capacity, contacting the fused DEL DNA molecule:DNA solubilizer molecule with an organic solvent and testing the solubility of the fused DEL DNA molecule:DNA solubilizer molecule in the organic solvent. Exemplary organic solvents that can be tested using the assay system of the invention include, but are not limited to DMSO, DMF, DMA, 1,4-dioxane, ACN and DCM.

In one embodiment, the invention provides method of generating a DEL containing compounds that require an organic solvent, the method comprising fusing a DEL DNA molecule to a DNA solubilizer molecule comprising a DNA blocker linked to a compound to increase the solubility of the DEL DNA molecule in the organic solvent of interest, contacting the fused DEL DNA molecule:DNA solubilizer molecule with the organic solvent, performing the chemical reaction to attach the DEL DNA molecule to the compound that requires an organic solvent, and removing the DNA solubilizer molecule comprising the DNA blocker to allow for further ligation and labeling of the DEL DNA molecule. Exemplary organic solvents that can be used include, but are not limited to DMSO, DMF, DMA, 1,4-dioxane, ACN and DCM.

Exemplary chemical reactions that can be performed to attach compounds to a DEL library include, but are not limited to, amidation.

### DNA Durability Assay

The invention is based, in part, on the development of an assay to determine the durability of nucleic acid molecules in various environments or conditions. Factors or conditions that can be tested for their impact on DNA durability using the assay system of the invention include, but are not limited to, organic solvents, buffers, high temperatures, altered pH, metal catalysts, metal scavengers, nucleotide content (e.g., % GC content), chemical ligands and other reagents.

In one embodiment, the invention provides an assay system to identify DNA conditions or reagents that can alter the durability of DNA molecules and DEL. In some embodiments, the DNA durability assay includes the steps of: covalently linking the two strands of a short oligonucleotide molecule to be tested with a spacer that carries a free functional group for chemical addition and denaturing the dsDNA molecule to generate a linked ssDNA molecule, contacting the test oligonucleotide with one or more condition or reagent to be tested for its effect on DNA durability, and analyzing the effect of the condition or reagent on the durability of the short oligonucleotide molecule.

In some embodiments, the method further includes a step of precipitating the short oligonucleotide molecule prior to analyzing the effect of the condition or reagent on the durability of the molecule. In some embodiments, the method of precipitating the short oligonucleotide molecule includes ethanol precipitation.

In some embodiments, the method of analyzing the effect of the condition or reagent on the durability of the short oligonucleotide molecule is performed using gel electroporation, LCMS, or any combination thereof.

In some embodiments, the invention relates to the use of a condition or reagent is identified according to the method of the invention of increasing DNA durability to generate a DNA DEL. In some embodiments, the invention provides for a DEL with increased durability.

### DEL Library

DEL allows the synthesis and screening of millions, or even billions, of encoded compounds cheaper and quicker than using conventional methods. This technology connects the disciplines of molecular biology and organic chemistry through the use of synthetic chemistry cycles to introduce diverse small molecule building blocks (BBs) encoded by unique DNA tags. Several cycles of affinity selection, typically involving an immobilized target protein and a library or a pool of libraries, yield a mixture of compounds enriched in binders to the protein of interest. Amplification of the DNA region by polymer chain reaction methods and posterior next generation sequencing permits the identification of the structure of the binding molecules. In one embodiment, one or more oligonucleotide tag molecule of the invention can be used in the de novo synthesis of a DEL library to provide a unique barcode sequence to assist in deconvolution of the data generated from the DEL. In one embodiment, one or more oligonucleotide tag molecule of the invention can be used to modify a pre-existing DEL library to provide a unique barcode sequence to assist in deconvolution of the data generated from the DEL or to add one or more additional functionality to the DEL.

In one embodiment, one or more tag oligonucleotide molecule of the invention is used to tag nucleic acid molecules of a DNA encoded library (DEL). In one embodiment, SELDA is used to identify a set of tag oligonucleotides that can be used to tag different samples or sets of nucleic acid molecules or DELs that can then be pooled together prior to further analysis.

DEL libraries generated according to the methods of the invention can be used for binding experiments targeting a single target, in-situ screening and/or detection, intracellular screening, detection in complex systems by proximity ligation assay (PLA), binding experiments targeting 2 or more targets simultaneously, and applications for single cell screening.

In some embodiments, one or more DEL are sequenced and the tags are subsequently used to associate a sequence DEL with the sample from which it derived. In various embodiments, the sequencing can be accommodated by Illumina, Applied Biosystems, Roche, and other deep sequencing technologies. Hybridization-based detection platforms could also be used but provide less resolution.

In some embodiments, multiple DEL are prepared in parallel and then pooled to generate a high throughput assay. For example, parallel assays may be carried out in a multi-well plate, such as a 96-well plate or a 384 well plate. The number of pooled samples is not necessarily limited as the limiting factors are 1) the number of oligonucleotide tags, or barcodes, used and 2) the number of sequencing reads desired per sample for a given sequencing platform. Therefore, the method may be extended to include more samples at a cost of reduced sequencing read coverage per sample.

In one embodiment, the invention provides oligonucleotide molecules having high ligation efficiency that can used for labeling a DEL. In some embodiments, the barcode sequence may be generated from ligation of at least one oligonucleotide molecule having high ligation efficiency to a de novo or pre-existing DEL. In one embodiment, the barcode sequence may be generated from step-wise ligation of at least two, three, four, five or more than five oligonucleotide molecules having high and/or comparable ligation efficiency to a de novo or pre-existing DEL. Exemplary oligonucleotides that can be ligated include, but are not limited to, the oligonucleotides set forth in Table 1. For example, in one embodiment, the barcode sequence results from stepwise ligation of oligonucleotides, wherein the stepwise ligation includes ligation of a first tag oligonucleotides from tag set #1 of Table 1, ligation of a second tag oligonucleotide from tag set #2 of Table 1 and ligation of a third tag oligonucleotide from tag set #3 of Table 1. In one embodiment, ligation of each tag occurs during the first step of a round of a multi-round split and pool protocol for generating a DEL.

In one embodiment, following ligation of a DEL to an oligonucleotide tag, the tagged DEL is ligated to a DNA blocker molecule comprising a restriction enzyme recognition site for a restriction enzyme that will generate a cut leaving an overhang that can be used for a subsequent ligation reaction. In one embodiment, the blocked DEL is then contacted with a restriction enzyme which cuts the tagged DEL to produce a single-stranded DNA overhang which can be used for ligation of another DNA molecule (e.g. another tag for the generation of a multi-tag barcode).

Exemplary restriction enzymes that can be used include, but are not limited to Pac1, Pme1, Sfi1, Asc1, EcoR1, Hind111, and Bsa1. Therefore, in one embodiment, the DNA blocker comprises a recognition site for one or more of Pac1, Pme1, Sfi1, Asc1, EcoR1, Hind111, or Bsa1 and the ligated product formed from ligation of the DNA blocker to the tagged DEL comprises a cleavage site for one or more of Pac1, Pme1, Sfi1, Asc1, EcoR1, Hind111, or Bsa1.

In one embodiment, the DNA blocker molecule further comprises a free chemical group such as a free functional amine. In one embodiment, the free chemical group can be functionalized with a solubilizing molecule such as PEG or a PEGylated molecule to alter the solubility of the DEL, thus increasing the solubility of the DEL in organic solvents including, but not limited to, DMSO, DMF, DMA, 1,4-dioxane, ACN and DCM. In some embodiments, the PEG comprises a molecular weight of at least 1,000; 2,000; 3,400; 5,000; 10,000; 20,000 or more than 20,000. In one embodiment, the solubilizing molecule comprises approximately 20 to 450 PEG units. In one embodiment, the solubilizing molecule comprises PEG MW 5,000. In one embodiment, the solubilizing molecule comprises activated polyethylene glycol mPEG-SCM (PEG-NHS ester, molecular weight of PEG=5,000).

In some embodiments, the DEL may further be ligated to one or more additional oligonucleotide sequences. For example, in some embodiments a DEL may be ligated to an oligonucleotide sequence comprising a restriction enzyme site, a spacer sequence, a nucleic acid molecule conjugated to a molecule for binding or purification including, but not limited to, a DNA-biotin conjugate, a DNA-magnetic bead conjugate, a DNA-antibody conjugate, or a DNA-nanobody conjugate, a nucleic acid molecule conjugated to a molecule for visualization including, but not limited to, a DNA-fluorophor conjugate, and a DNA-quantum dot conjugate, a nucleic acid molecule conjugated to a molecule for cell permeabilization including, but not limited to, a DNA-cholesterol conjugate, a DNA-polyArg conjugate, and a DNA-TatSeq conjugate, or a nucleic acid molecule conjugated to a molecule for proximal PCR or rolling circle amplification (RCA).

In one embodiment, the invention provides methods for modifying an existing DEL comprising incorporating a unique restriction site on at least one side of the DNA tags of an existing DEL. In one embodiment, the DEL can then be further modified to incorporate one or more of a tag oligonucleotide, a spacer sequence, a nucleic acid molecule conjugated to a molecule for binding or purification including, but not limited to, a DNA-biotin conjugate, a DNA-magnetic bead conjugate, a DNA-antibody conjugate, or a DNA-nanobody conjugate, a nucleic acid molecule conjugated to a molecule for visulaization including, but not limited to, a DNA-fluorophor conjugate, and a DNA-quantum dot conjugate, a nucleic acid molecule conjugated to a molecule for cell permeabilization including, but not limited to, a DNA-cholesterol conjugate, a DNA-polyArg conjugate, and a DNA-TatSeq conjugate, or a nucleic acid molecule conjugated to a molecule for proximal PCR or rolling circle amplification (RCA).

### Nucleic Acid Samples and Preparation

As contemplated herein, the present invention may be used in the preparation and analysis of DEL libraries. The DEL library may be prepared (e.g., library preparation) in any manner as would be understood by those having ordinary skill in the art. While there are many variations of library preparation, the purpose is to construct nucleic acid fragments of a suitable size for a sequencing instrument and to modify the ends of the sample nucleic acid to work with the chemistry of a selected sequencing process. Depending on application, nucleic acid fragments may be generated having a length of about 25 to about 1000 bases. It should be appreciated that the present invention can accommodate any nucleic acid fragment size range that can be read by a sequencer. This can be achieved by selecting primers such that the resulting PCR product is within the desired range specific for the sequencer and sequencing method desired. For example, in various embodiments a desired PCR fragment size, including barcode and adaptor regions is about 100, 150, 200, 250, 300, 350, 400, 450 or about 500 bp. Both the 5′ and 3′ ends of the PCR products comprise nucleic acid adapters. In various embodiments, these adapters have multiple roles, such as allowing attachment of the specimen strands to a substrate (bead or flow cell) and having a nucleic acid sequence that can be used to initiate the sequencing reaction through hybridization to a sequencing primer. Further, in some embodiments, the PCR products also contain unique sequences (bar-coding) that allow for identification of individual samples in a multiplexed run. The key component of this attachment process is that each individual PCR product is attached to a bead or location on a slide or flow cell. This single PCR fragment can then be further amplified to generate hundreds of identical copies of itself in a clustered region on the bead, flow cell or slide location. These clusters of identical DNA form the product that is sequenced by any one of several next generation sequencing technologies.

The samples can be sequenced using any massively parallel sequencing platform. Non-limiting examples of sequencers include Illumina/Solexa GAII, AB SOLiD system, Ion Torrent PGM, Ion Proton, Illumina MiSeq, Illumina HiSeq 2000 or 2500 and the like.

As contemplated herein, the present invention includes methods of analyzing Next Gen Sequencing data. Generally, sequence reads are aligned, or mapped, to a reference sequence using, for example, available commercial software or open source freeware (e.g., nucleotide and quality data input, mapped reads output). This may include preparation of read data for processing using format conversion tools and optional quality and artifact removal filters before passing the read data to an alignment tool. Next, variants are called (e.g., summarized data input, variant calls output) and interpreted (e.g., variant calls input, genotype information output).

Standard approaches to mapping and analysis of this type of massively parallel sequence data are applicable to the invention described herein. In some embodiments, an analytical pipeline may detect the binding sites of a protein of interest, as outlined in the method below. First, raw read data, which may include sequence and quality information from the sequencing hardware, is received and entered into the system. The data is optionally prefiltered, for example, one read at a time or in parallel, to remove data that is too low in quality, typically by end trimming or rejection. For a multiplexed sequencing reaction, the raw reads are sorted according to the barcode region to group reads from each individual sample. The reads are then trimmed to remove barcode and adaptor sequences.

The remaining data is then aligned using a set of reference sequences. Read data can be mapped to reference sequences using any mapping software, and using appropriate alignment and sensitivity settings suitable for the goal of the project. Mapped reads may optionally be postfiltered to remove low quality or uncertain mappings. The total numbers of aligned reads can be determined using any appropriate method including, but not limited to, SAMtools, a PERL script, a PYTHON script, and a sequencing analysis pipeline.

In various embodiments, at least 1000, at least 2000, at least 3000, at least 4000, at least 5000, at least 10,000, at least 50,000, at least 100,000, at least 500,000 or more than 500,000 sequencing reads are determined to be ‘high quality’ after passing quality filters. In one embodiment, ‘high quality’ sequencing reads are aligned to one or more reference sequences.

### Kits

In one embodiment, the invention provides a kit for use in the DNA ligation assay of the invention. In one embodiment, the kit comprises one or more of: (a) a substrate for attaching a first accessory oligonucleotide or capture oligonucleotide; (b) a first accessory oligonucleotide or capture oligonucleotide comprising a moiety for attaching the a first accessory oligonucleotide or capture oligonucleotide to the substrate; and (c) a detectable moiety for detection of the ligation between the first accessory oligonucleotide or capture oligonucleotide and one or more test nucleic acid molecules or intermediary test oligonucleotides.

In one embodiment, the kit comprises one or more of: (a) a substrate for attaching a first accessory oligonucleotide or capture oligonucleotide; (b) a first accessory oligonucleotide or capture oligonucleotide comprising a moiety for attaching the a first accessory oligonucleotide or capture oligonucleotide to the substrate; (c) a second accessory oligonucleotide comprising a detectable moiety for detection of the ligation between the first accessory oligonucleotide or capture oligonucleotide, an intermediary target oligonucleotide and the second oligonucleotide.

In one embodiment, the invention provides a kit for use in labeling a DEL. In one embodiment, the kit comprises at least 1, 2, 3, 4, 5, 6, 7, 8, 9, 10, 11, 12, 13, 14, 15, 16, 17, 18, 19, 20 or more than 20 tag oligonucleotides identified using the DNA ligation assay method of the invention. Exemplary tag sets that can be included in the kit of the invention include, but are not limited to, the series 1, series 2 or series 3 tag sets as set forth in Table 1.

Any kit of the invention may also include suitable instructional material, storage containers, e.g., ampules, vials, tubes, etc., for each reagent disclosed herein, an reagents used as controls, e.g., a positive control nucleic acid sequence or positive control antibody). The reagents may be present in the kits in any convenient form, such as, e.g., in a solution or in a powder form. The kits may further include a packaging container, optionally having one or more partitions for housing the various reagents.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Method to Effectively Measure, Optimize and Calibrate DNA Ligation Efficiency—Miniaturization Compatible with Multi-Well Plate Testing

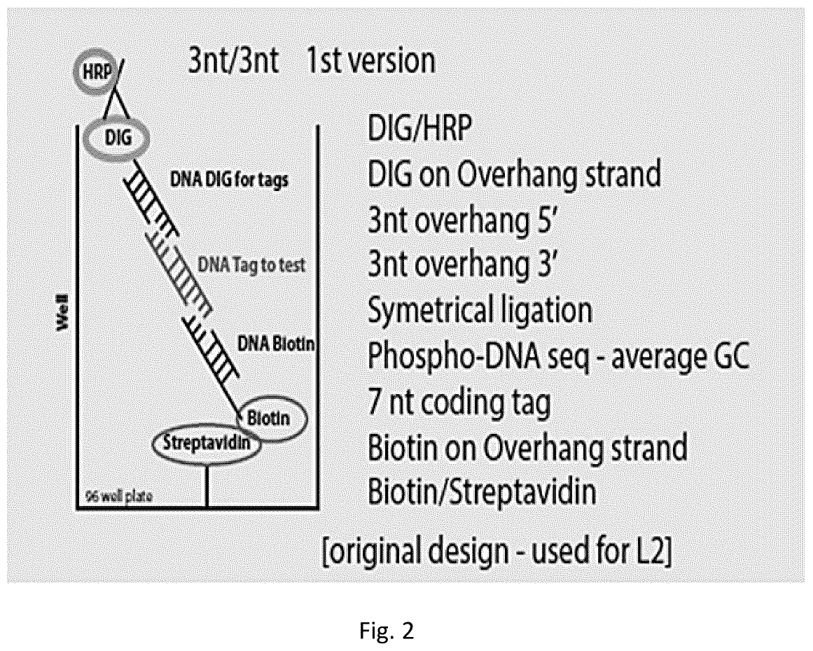

Several categories of parameters have to be taking into account when ligating DNA fragments such as: a) design of the DNA fragments, b) purity of DNA fragments following synthesis, c) concentration (that will dictate the relative abundance), d) ligation efficiency (that will dictate the relative abundance), e) conservation and durability over time, and f) impact of chemical reactions performed in presence of DNA. A general presentation of the kit system is shown in FIG. 1 and FIG. 2.

Testing and evaluating the quality of novel DNA fragments by measuring their ligation efficiency effectively in a multi-well (e.g. 96-wells) format. Only satisfying tags are then used and they are incorporated into libraries at the exact same relative ratio. The entire DEL technology is based on the use of DNA tags to barcode organic compounds in order to be able to identify them later by decoding the DNA sequence. Therefore, DNA tags are extremely important and can greatly affect the overall quality of a DEL, as well as the resulting screening campaigns. For example, the relative abundance of each DNA tags (which is assumed to be identical for all DNA tags from a theoretical point of view) and their relative ligation efficiency, are crucial for decoding purposes. The more disperse the relative representation of the tags among themselves in a given library, the more difficult it will be to interpret a screening campaign, to separate real hits from background, to perform enrichment analysis. Normalizing tags beforehand based on their individual and actual ligation efficiency annihilate disparities and greatly facilitate the decoding step, the drug discovery process. For example, 2% of defective tags on a 500×500×500 tags library would mean that over 2.5 million compounds (2,505,010 precisely) will not be represented.

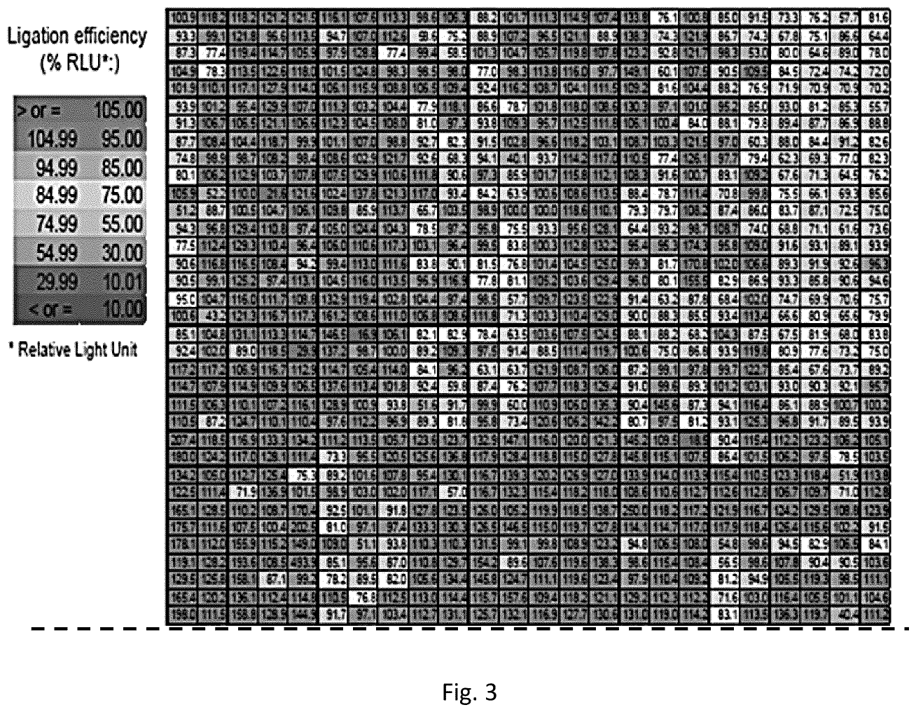

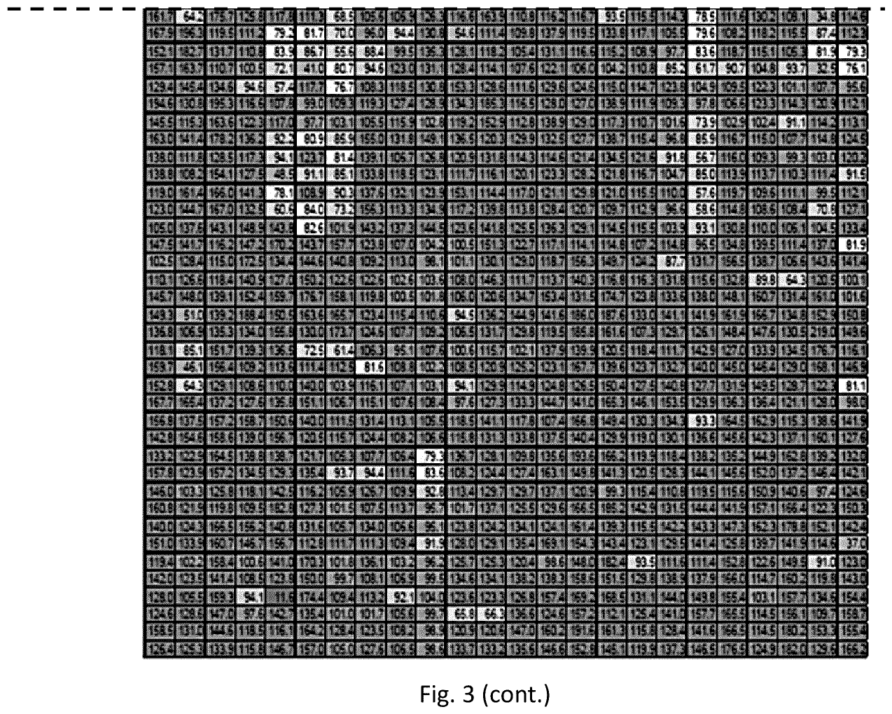

1,700 different tag sequences have been tested, proving the need to actually test the tag for ligation efficiencies on both ends. FIG. 3 represents a visual summary with a gradient map of the perfect tags (dark green) and poor tags (yellow to red). By using tags without this quality test would have significantly reduced the overall quality of the library by impacting on the presence and on the relative abundance of many tags.

Parameters that can be quantified by measuring ligation efficiency:
a) Design of the tags (e.g. nature of the sequence itself, length, number/nature of overhang nucleotides, natural DNA versus non-natural DNA nucleotides, etc. . . . ),
b) The synthesis and purification of the actual tags (appropriate presence of overhang nucleotides, phosphate group, appropriate purification level that is compatible with DNA ligation)
c) Actual functional concentration in solution as general DNA quantification methods are not reliable for very small fragments and do not take into account efficiency
d) Durability over time [e.g. using or re-using DNA tags after yearlong storage]. Each of these parameters can greatly affect DNA tag's quality and ligation efficiency. Even if some of the parameters involved can be partially controlled, there are a number of conditions that can affect the overall results. Therefore, instead of normalizing each parameter individually, we propose a method to quickly test and normalize each tag based on the end result: DNA ligation efficiency. By testing several hundreds of tags using our high-throughput assay kit, we were able to demonstrate this intrinsic variability. It became necessary to find a systematic method to not only correct this but also to be able to identify DNA tags that would have to be discarded because of poor or null ligation efficiency.

Details about of relevant parameters (FIG. 1):
a) DNA tags Design and requisites
   Length (e.g. number of nucleotides)
   Composition of the nucleotide sequence (GC content, nature of 3′ nucleotide, nature of 5′ overhang)
   Unwanted formation of single strand hairpins
   Nature of the nucleotide sequence such as natural DNA vs. non-natural DNA (e.g. LNA for Locked nucleic acid)
   Length of overhang ends (e.g. 1 nucleotide, 2 nucleotides, etc.)
   Symmetry of the overhang ends (e.g. symmetrical [e.g. 3 nt-3 nt] versus asymmetrical [e.g. 2 nt-4 nt])
   Presence of a phosphate group on each side of the DNA fragment (in case of double strand) required for ligation.
b) Products obtained after DNA synthesis
   Ideally 100% of the actual synthetized product should match the design.
   Smaller DNA sequences (e.g. a few nucleotides) are difficult to quantify precisely by conventional methods (e.g. NanoDrop, PicoGreen, bioanalyzer, spectrometry).
   Quality of the overhang ends: after DNA synthesis, resuspension and possibly storage, all nucleotides (especially the single strand nucleotides necessary for cohesive ligation) must remain present at all times.
   Presence of a Phosphate group required for ligation on each 3′ strand of DNA fragments to be ligated.
   Annealing of the two complementary DNA strands should be complete but cannot be adequately verified on such short DNA sequences.
   Hairpins undetected during the design step will not be detected after annealing.
c) Actual concentration Depending on the buffer, hygroscopy and solubility can affect the final concentration and should be taking into account by normalization.
d) DNA conservation/durability
   Quality of the DNA strands after synthesis and over time—all nucleotides, especially overhang nucleotides, must still be present for effective DNA ligation.
   Presence of a Phosphate group is required for DNA ligation on each 3′ strand for each DNA fragment.
      Testing Chemically Treated DNA Fragments for Ligation Efficiency for Example for Use in DNA-Encoded Library Construction to Ensure Efficient Ligation Capacity after Chemical Reactions.

The DNA-encoded library technology is based on the continuous alternation between molecular biology and organic chemistry. The impact of every chemical reaction on DNA has to be tested. Once ligated onto a lengthening DNA label, DNA fragments are subjected to solvents/reagents/catalysts (e.g. DMSO, DMF, base solutions, copper) and chemical conditions (e.g. high temperatures, range of pH, buffers) that are not typically DNA friendly solutions/conditions. It is important to ensure that all of the above parameters regarding DNA fragments remain intact after chemical reactions are performed. This 96-well format ligation efficiency kit allows for the measurement of the impact of such treatments on DNA ligation efficiency, in the right DEL context.

Test DNA Fragments for Ligation for Example for Use in DNA-Encoded Library Construction to Ensure Efficient Ligation Capacity—and Follow Up with PCR and DNA Sequencing

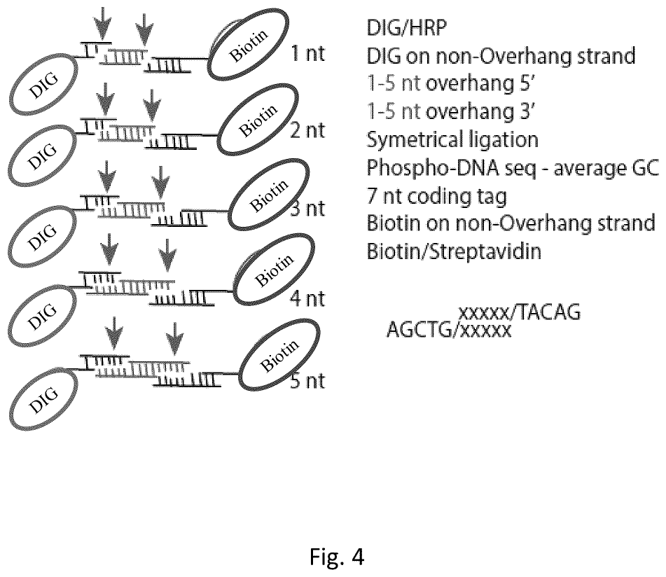

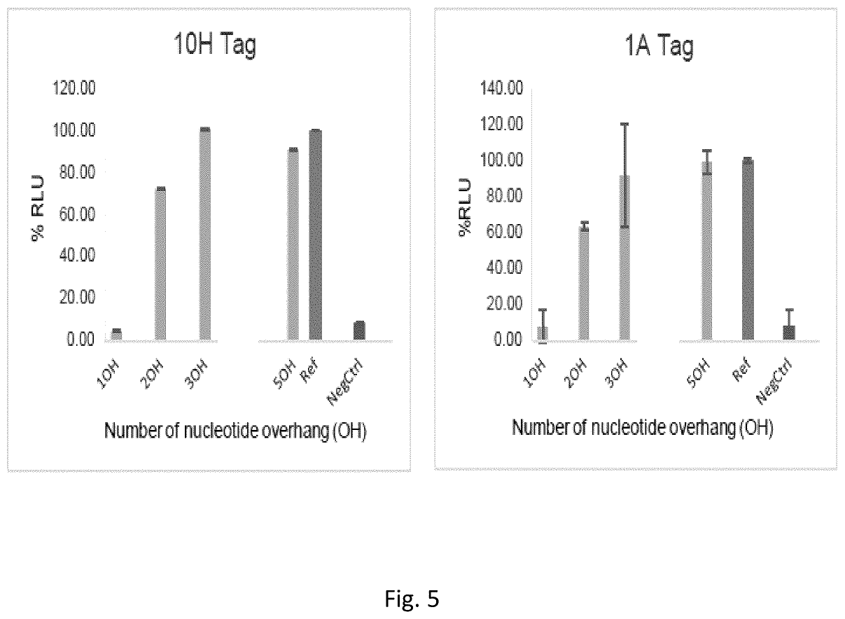

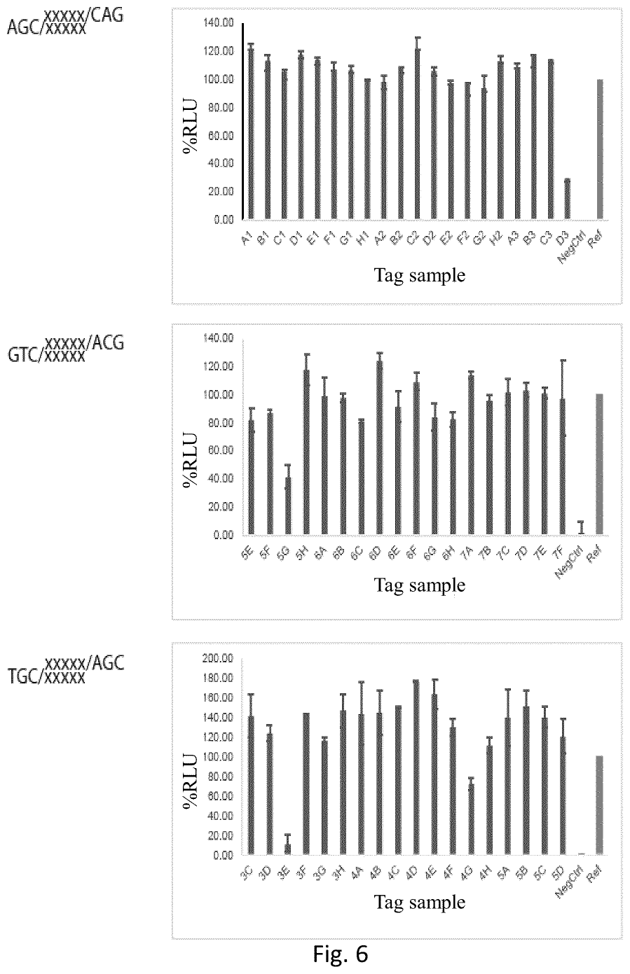

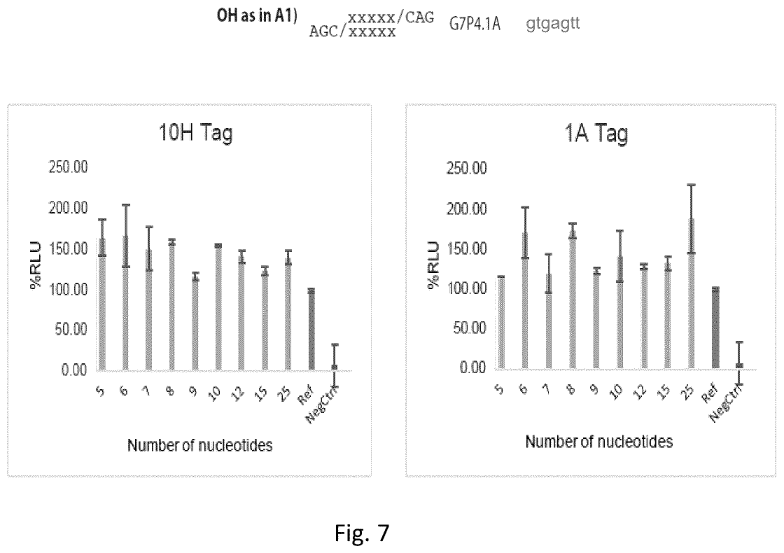

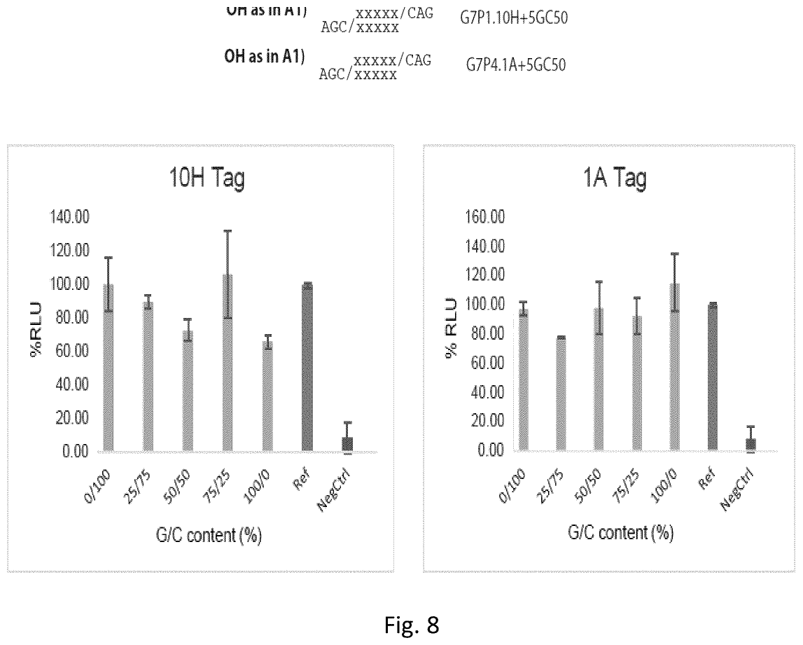

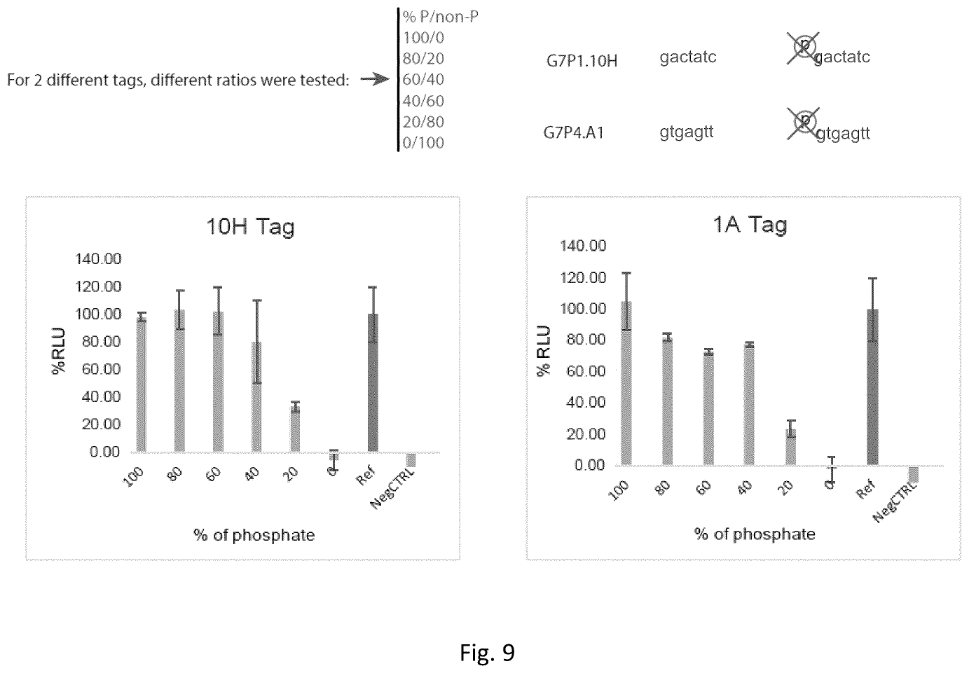

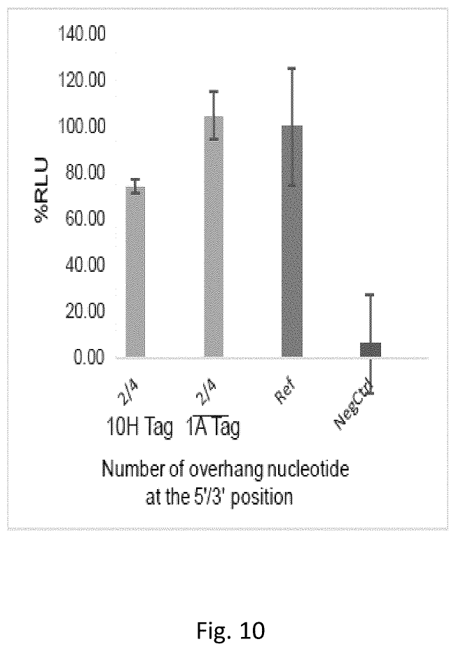

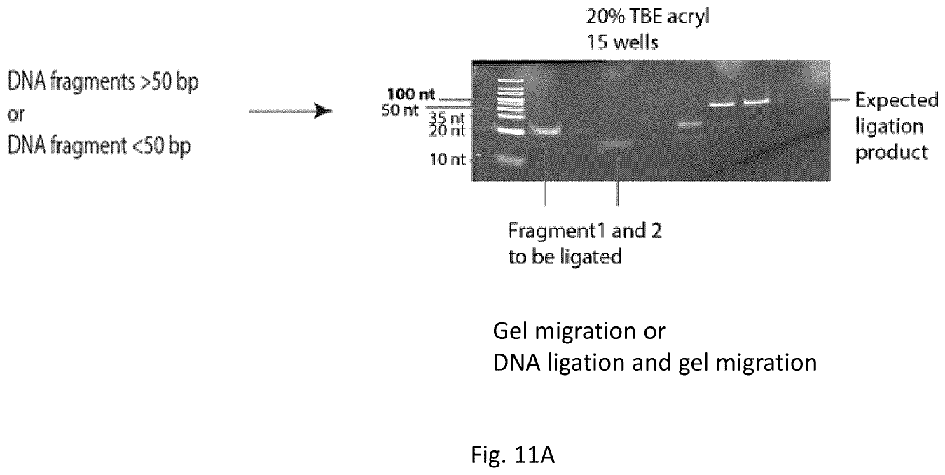

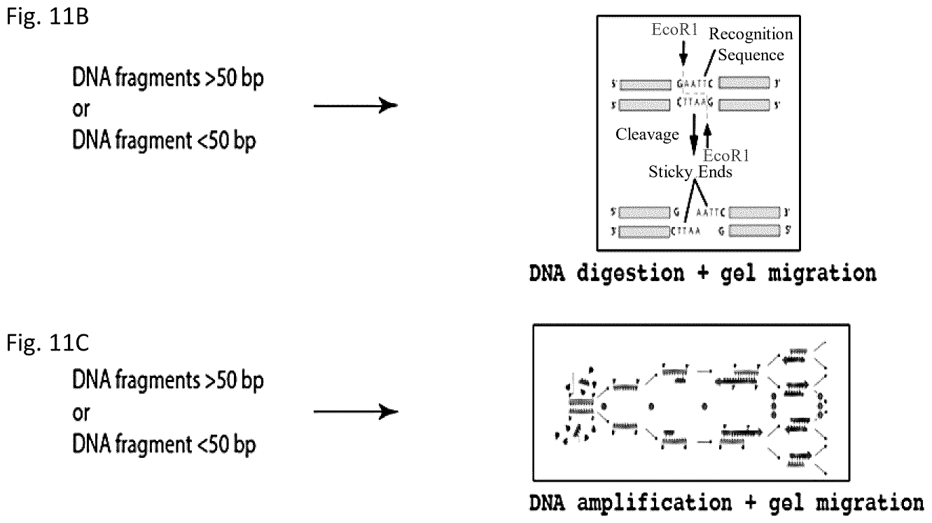

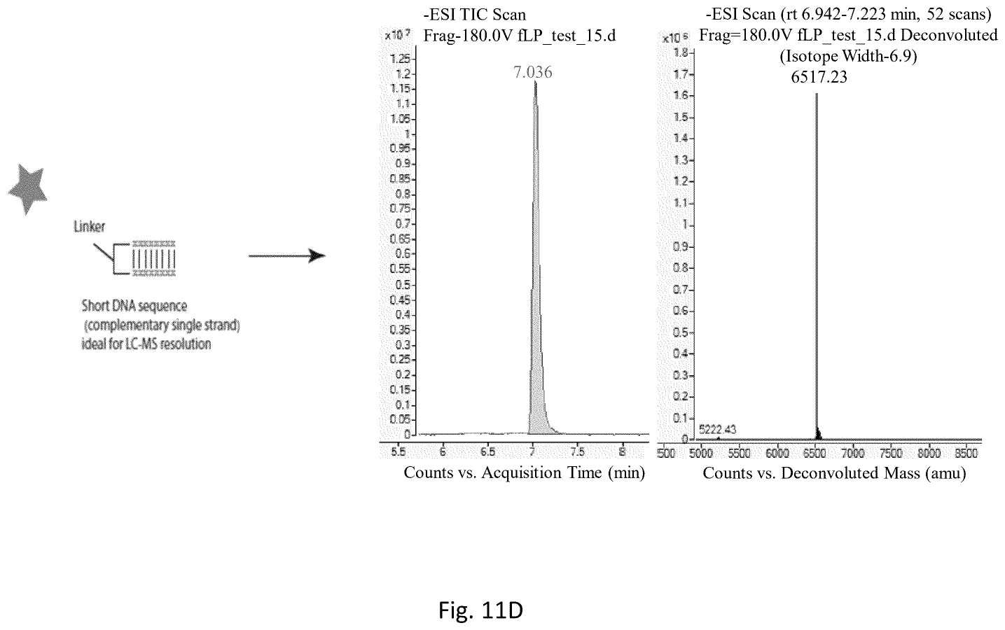

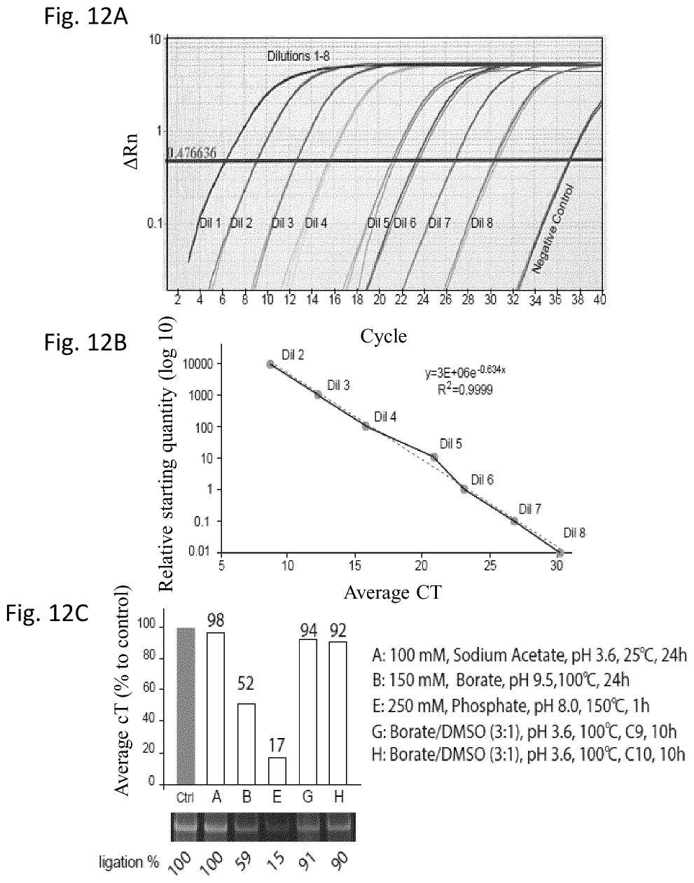

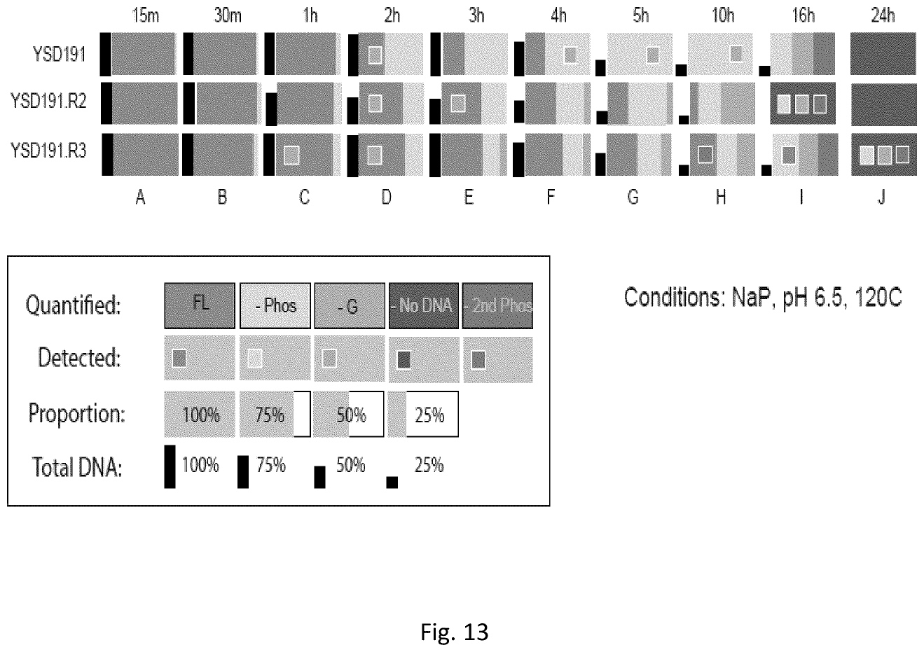

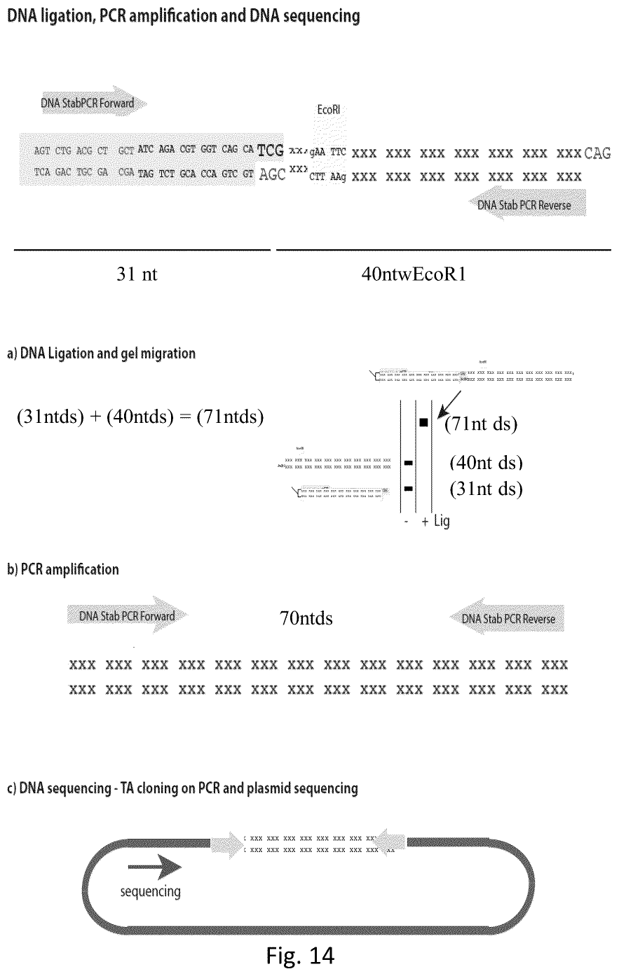

A system to allow test ligation has been designed and tested to measure quickly the ligation capacity of a DNA fragment.
a) Different overhang lengths have been tested. FIGS. 4 and 5 demonstrate that using 1, 2, 3, and 5 nucleotide overhangs work. However in the conditions used (1 hour of incubation for DNA ligation) the 1 overhang nucleotide was not very efficient.
b) Different overhang sequences have been tested. FIG. 3 demonstrates the results of testing 6×96 Tags with same 2 overhang sequences (Tag1), 6×96 tags with same 2 overhang sequences (Tag2), and 6×96 tags with same 2 overhang sequences (Tag3).
c) Different lengths of tag have been tested. FIG. 7 demonstrates the results using 4, 5, 6, 7, 8, 9, 10, 12, 15 and 25 nucleotides long DNA Tag sequences.
d) Different GC compositions (0 to 100%) have been tested. FIG. 8 demonstrates results with 0, 25, 50, 75 and 100% GC.
e) Asymmetrical ligations presenting different overhang lengths have been tested (FIG. 10). Specifically, 2 combinations have been tested with 2 overhang nucleotides on one side and 4 overhang nucleotides on the other side.
f) Different ratios of Phosphorylation have been successfully tested (FIG. 9), including 0%, 20%, 40%, 60%, 80% and 100% of phosphorylation. Significant deficiencies are detected at less than 60% phosphorylation. The ideal is 100%.
g) Strand choice for DIG and Biotin. FIG. 2 demonstrates that both strands work as well.

### Example 2: Evaluation of DNA Durability

Organic chemistry typically requires harsh conditions and non-aqueous solvents or organic solvents. Due to the relative fragility of the DNA, chemical conditions used in presence of DNA have to be mild (e.g. aqueous solution, non-extreme pH, non-acidic pH, temperature below 100 C). To evaluate the durability of DNA in novel chemical conditions it is necessary to have methods to determine and quantify the impact on DNA with a molecular resolution. DNA durability in this context is different from DNA stability. “Stability” in the DNA world describes the single strand/double strand nature of DNA. Here, it does not matter if DNA is single or double strand and durability refers to the integrity of DNA molecules (e.g. presence of all nucleotides, bases, phosphates).

Traditional DNA methods (e.g. gel migration, spectrometry-based methods for DNA quantification) are quick and practical for a number of applications but they do not allow for precise molecular resolution. For example, if the 3′ phosphate group is missing, or if a single nucleotide is missing after a given chemical reaction, this will not be visible nor quantifiable with regular methods. However, it is crucial that we have access to a method addressing that aspect very precisely, at the molecular level.

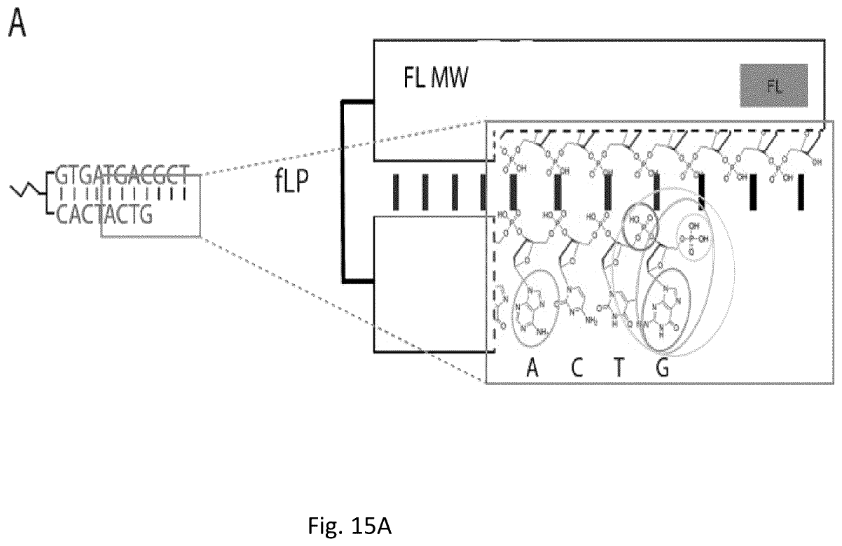

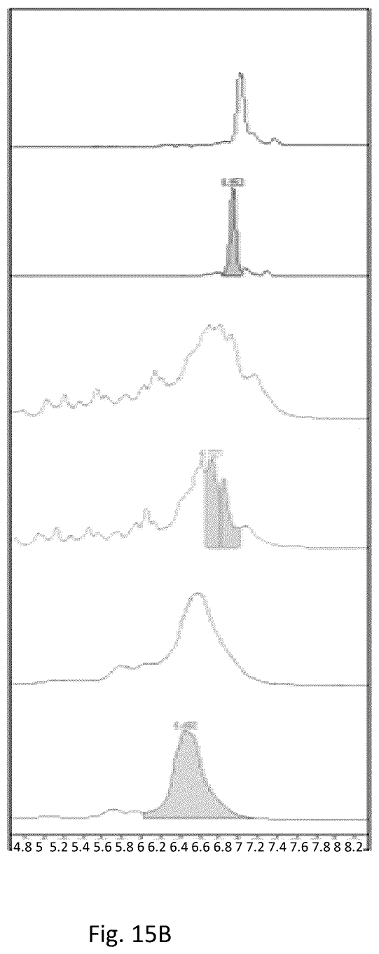

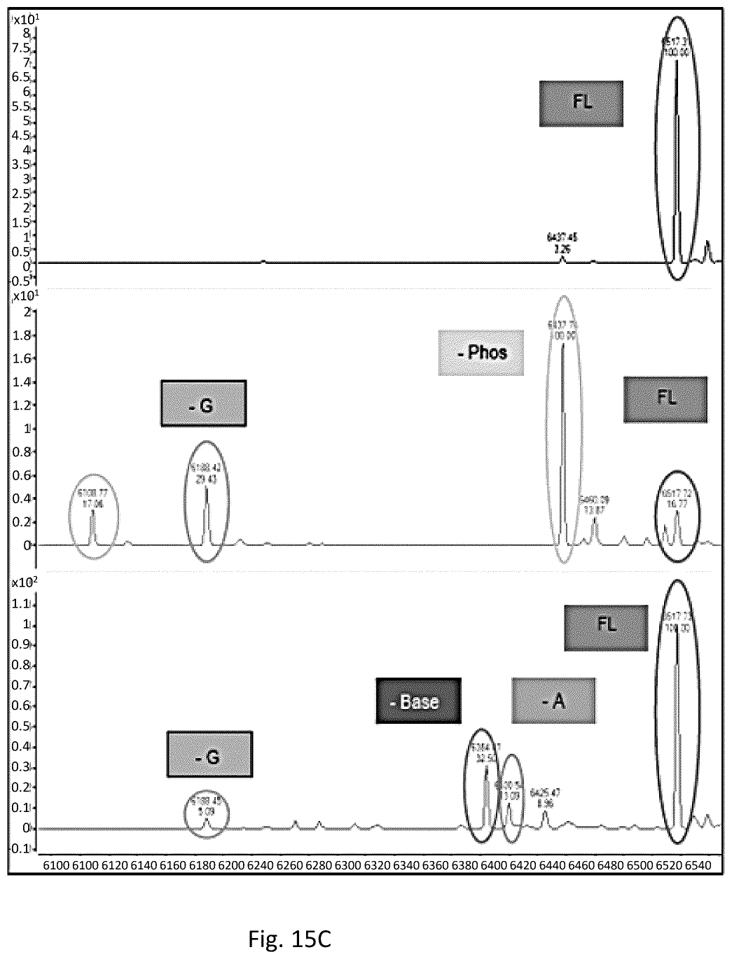

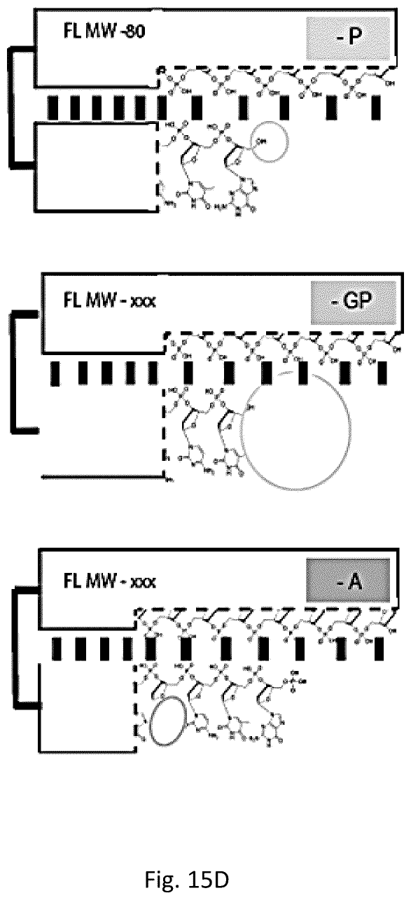

To take that into account, we are proposing to use liquid chromatography mass spectrometry (LC-MS). We demonstrated that by using a specific DNA fragment with the following characteristics the molecular resolution needed was reached and all needed parameters were addressed (FIG. 15):
2 complementary DNA strands covalently linked—they are linked so that entire molecule is seen, and not just 1 DNA strand.
Small enough to be compatible with a clear LC-MS resolution after deconvolution.
Large enough so that the fragment retains DNA properties such as the possibility to precipitate the DNA and also to be ligated to another DNA fragment.
3 single strand nucleotides for ligation purposes
The presence of a phosphate group for ligation purposes

All species of modified DNA can be observed:
the full-length fragment (FL) and its corresponding LC-MS peak is shown in green
the loss of the phosphate group (yellow) and its corresponding LC-MS peak is shown in yellow
the loss of the last base and its corresponding LC-MS peak is shown in light purple
the loss of an internal base and its corresponding LC-MS peak is shown in blue
the loss of an entire nucleotide and its corresponding LC-MS peak is shown in orange
the loss of an entire nucleotide plus the next phosphate group and its corresponding LC-MS peak is shown in pink.

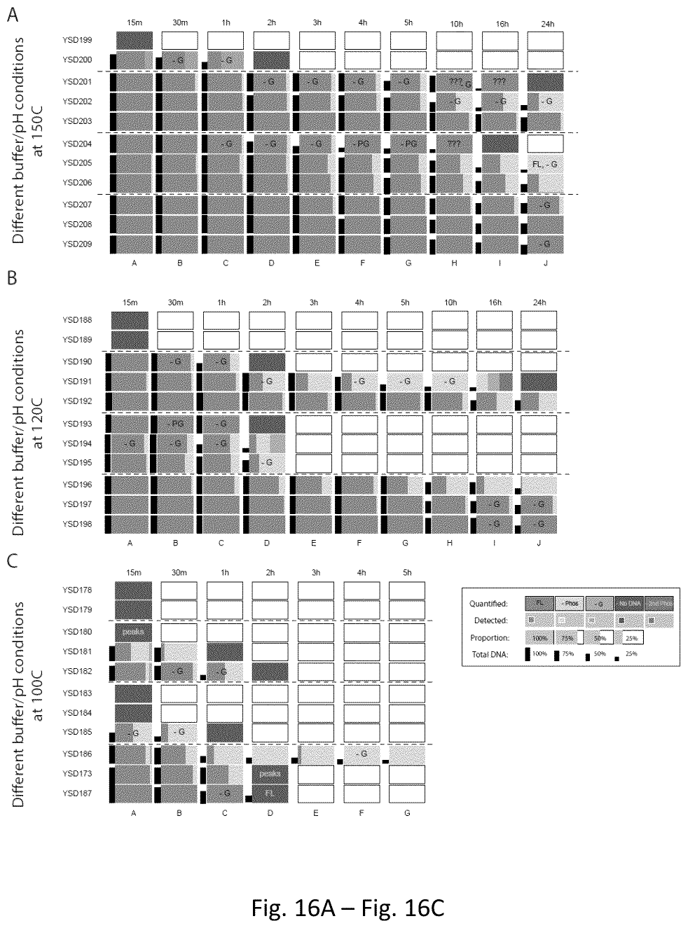

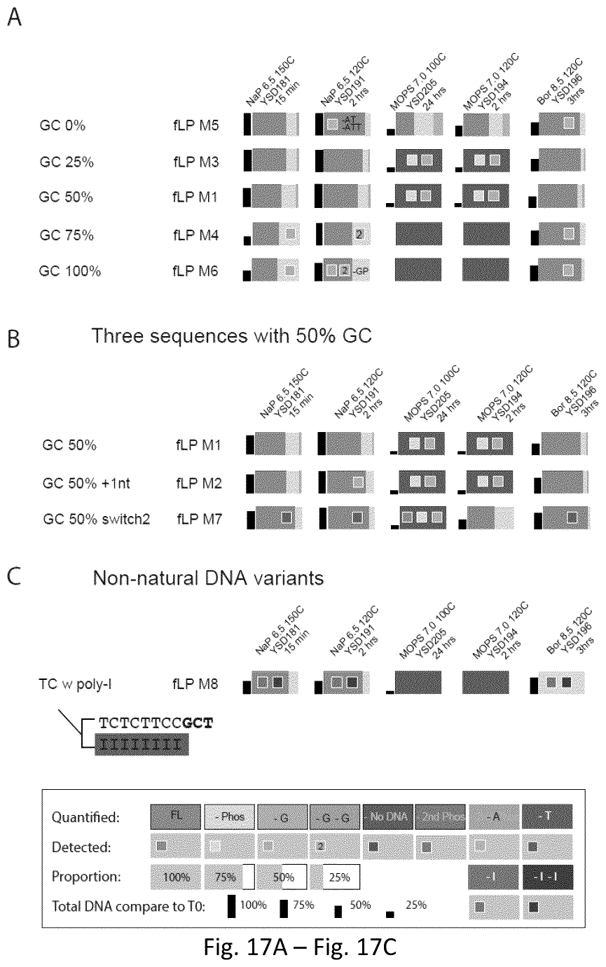

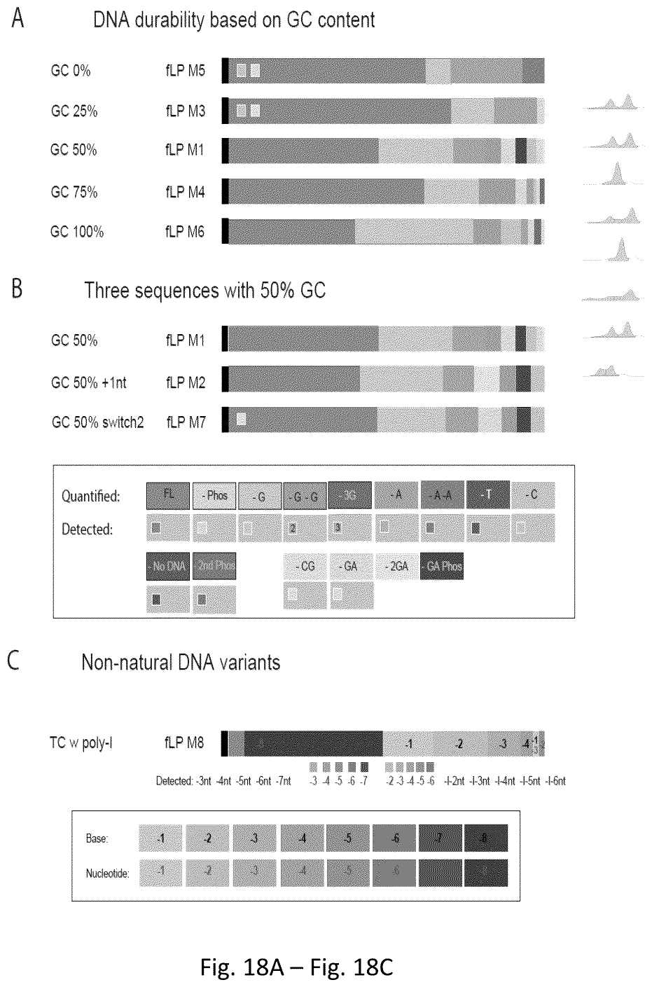

This system and this color-coded representation was used to test a large number of conditions as shown in FIG. 16-18. For example:
3 different temperatures (up to 150 C—when typically DEL chemistry stops at 100 C).
15 incubation times (15 minutes to 100 hours)
4 different buffers
pH ranging from 4.5 to 11
different GC contents (0% to 100%)
different sequences with same GC content
non-natural DNA
8 catalysts never used in the DEL field
2 solvents never used in the DEL field
2 bases never used in the DEL field

In summary, multiple parameters were tested to evaluate precisely if these parameters are compatible with the presence of DNA and if they could be used for chemical modifications.

### Example 3: Method to Enhance Capabilities of Existing DNA-Encoded Libraries Post-Synthesis

Today DEL libraries are used only for binding experiments typically using 1 isolated target. However, other uses and different read-outs are envisioned, including in-situ screening and/or detection, intracellular screening, detection in complex systems by proximity ligation assay (PLA), the targeting of 2 or more targets simultaneously, applications for single cell screening, etc.

It is time consuming and expensive to build an entire new DEL libraries from scratch. Therefore, this invention focuses on expanding the versatility of DEL libraries by incorporating a feature (unique restriction site on one side of the DNA tags) that allows for modification of a DEL library, or a fraction of it, after completion, even years later while keeping the flexibility of the type of modification and also the possibility to further modify the modified library.

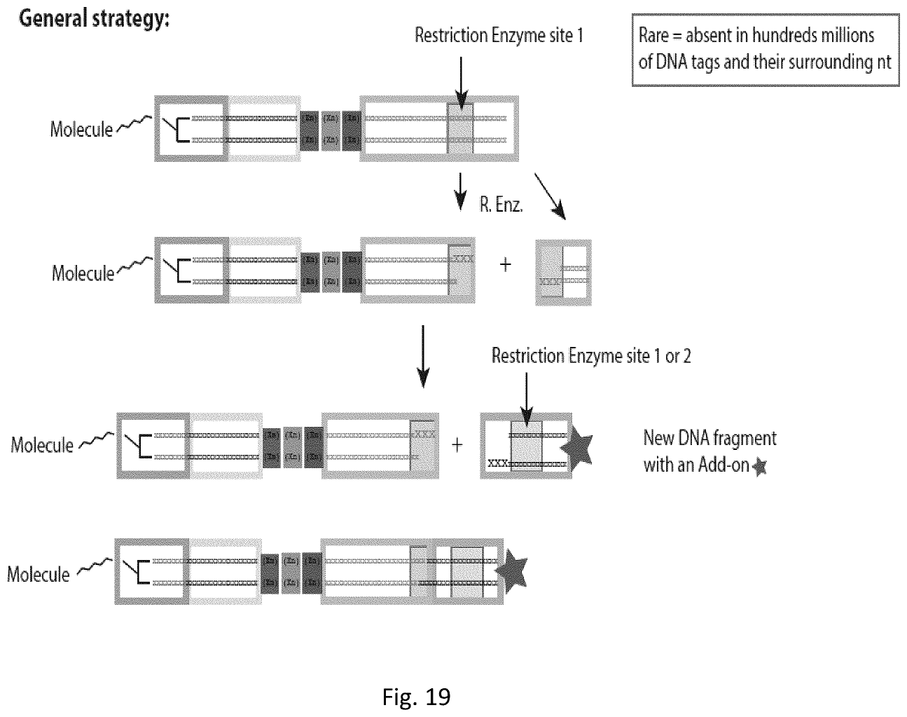

By incorporating a unique and/or rare enzyme restriction site one one side of a DEL DNA label (FIG. 19; pink box), new features can be added to an existing DEL library by ligating a novel piece of DNA harboring the desired modification, the “DNA modifier”. This is accomplished by digesting an already made DEL library (3′ end of all DNA tags all at once in a single tube) and by ligating the “DNA modifier” harboring the new feature(s) that is then added to every single DNA label all at once.

Some example of modifications (moieties) grouped in larger categories are presented below:
a) Binding/purification:
   DNA-Biotin
   DNA-Magnetic Beads
   DNA-Antibody
   DNA-nanobody
b) Visualization:
   DNA-Fluorescent molecule (e.g. FITC)
   DNA-Quantum-dot
c) Cell permeabilization:
   DNA-Cholesterol
   DNA-Poly-Arginine
   DNA-Tat/HIV Sequence
d) Proximal PCR/RCA or Proximity Ligation Assay:
   DNA-DNA single strand

Any restriction enzyme can be used as long as its restriction site is not present anywhere else in the DNA labels. Therefore, it is best to avoid promiscuous restriction enzymes that have a short recognition site (e.g. 4 nucleotides) and regular restriction enzymes (e.g. 6 nucleotides).

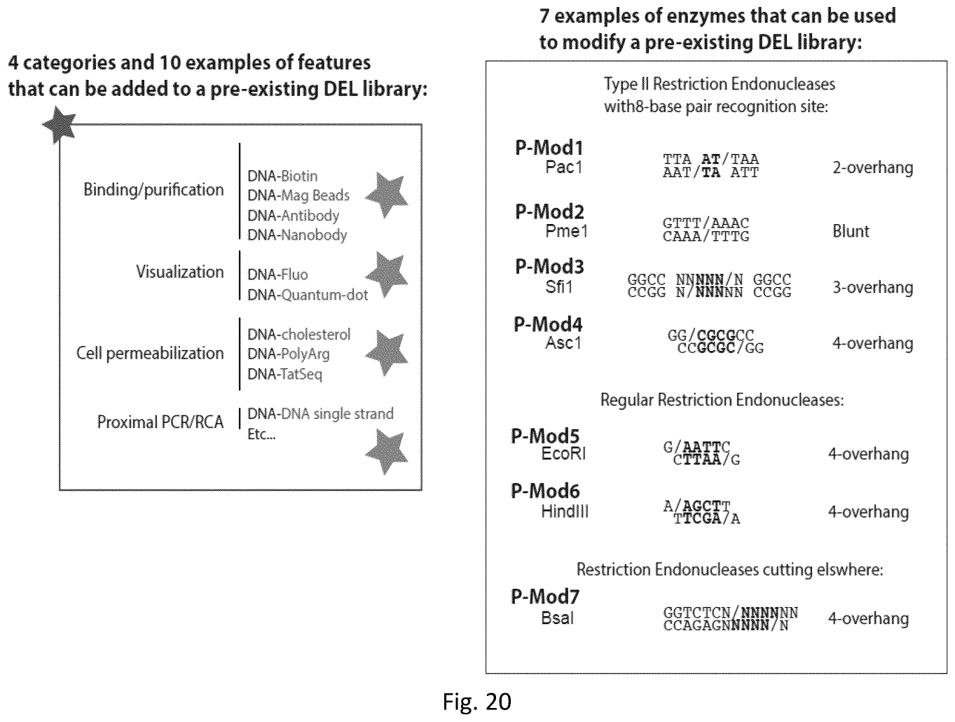

A number of examples of endonucleases are shown in FIG. 20. In any case, the chosen restriction enzyme recognition site is taken into account when designing any oligonucleotide or nucleic acid fragment entering in the construction of a DEL, considering the oligonucleotides or nucleic acid fragments themselves and also the junctions between 2 oligonucleotides or nucleic acid fragments, as well as the entire labels, to ensure that it remains absent from all the labels except where desired at the 3′ end.

As an exemplary experiment, a rare restriction enzyme is used that requires 8 exact nucleotides to recognize and cut DNA (e.g. Pac). This approach significantly decreases the probably of random cuts inside the labels at non-desired locations. The DNA labels are evaluated to ensure that the recognition sequence (e.g. Pac: TTAATTAA) does not exist in the DNA labels.

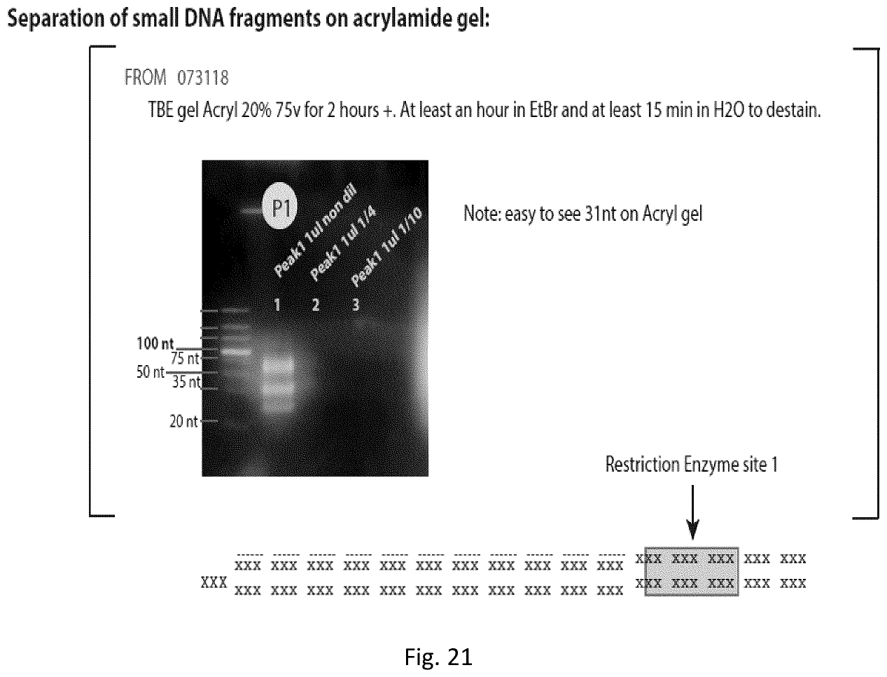

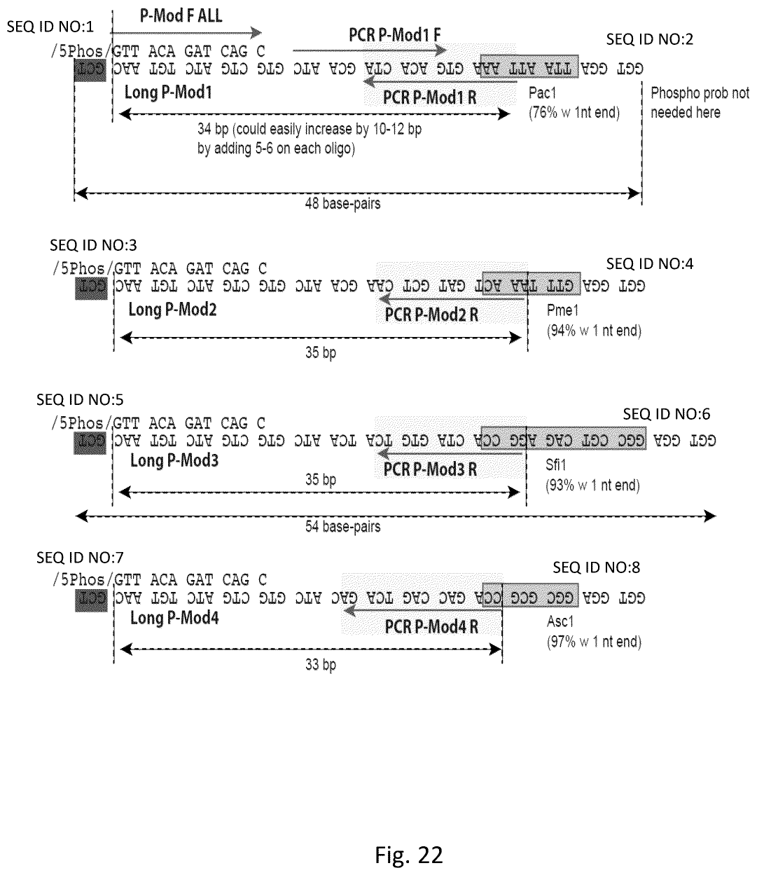

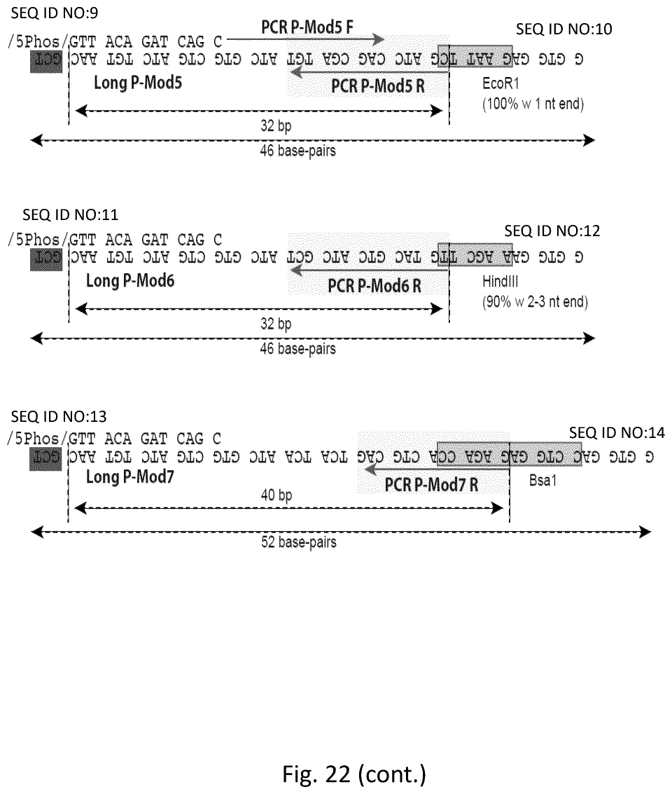

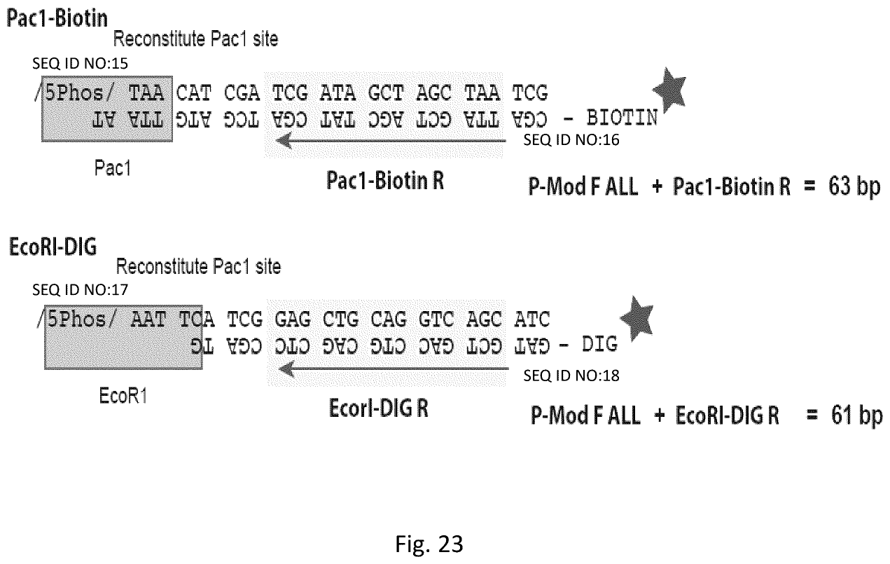

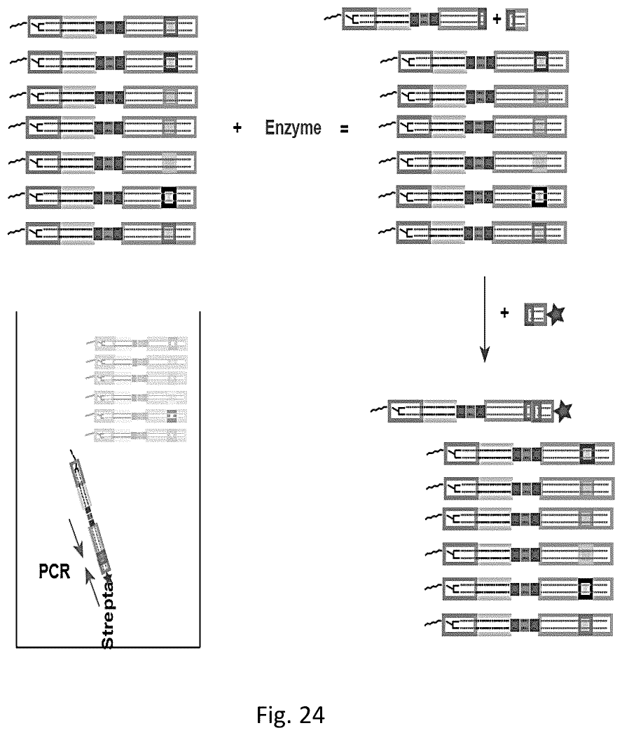

Experiments were designed to demonstrate that the strategy works and that the library is modified in a targeted/specific way (FIG. 21-FIG. 24) where only the targeted labels are modified, using a mix of 7 different DNA strands mimicking 7 DEL DNA labels. A DNA fragment harboring a Biotin moiety was used as a “DNA modifier”. The modified fragment was retained on the bottom of streptavidin coated wells using the streptavidin-biotin system. The specificity was confirmed by PCR, showing that only the targeted DNA fragment out of 7 remained present in the coated wells, indicating that the targeted DNA fragment, and only the targeted DNA fragment, was effectively modified with the biotin moiety, as expected.

The size of the DEL library does not matter (e.g. 1, 10 or 100 millions molecules library). Only the amount of DNA that has to be modified and its concentration are adjusted to perform the digestion with the appropriate restriction enzyme to ensure optimal digestion.

By adding another rare restriction site in the “DNA modifier” fragment to be added (e.g. the fragment with biotin in the example above), another opportunity to further modify the modified DEL library is created, and as long as the latest modifier harbors a rare restriction site, the existing DEL can be further modified.

Two or more moieties can be added on the “DNA modifier” fragment (e.g. biotin and fluorescent molecule such as Alexa488 or FITC; biotin and a single strand DNA; a fluorescent molecule such as Alexa488 or FITC and a single strand DNA; biotin and a quantum dot; etc.).

### Example 4: Method to Evaluate and Quantify the Use of Non-DNA Molecules for Encoded Libraries

DEL libraries are made of natural DNA. However, natural DNA comes with a number of constraints (e.g. temperature, solvent, pH, . . . ) that drastically limit the chemical space available for DEL. One possibility to increase DNA resistance is to use non-natural DNA molecules. Non-natural DNA might help increase durability and allow the use of chemical reactions that are harsher, consequently increasing the DEL-compatible chemical space. Some modification such as—phosphorothioate also increase the resistance to nucleases. This is an advantage in certain screening conditions including in-vivo, intracellularly, and screening on cells.

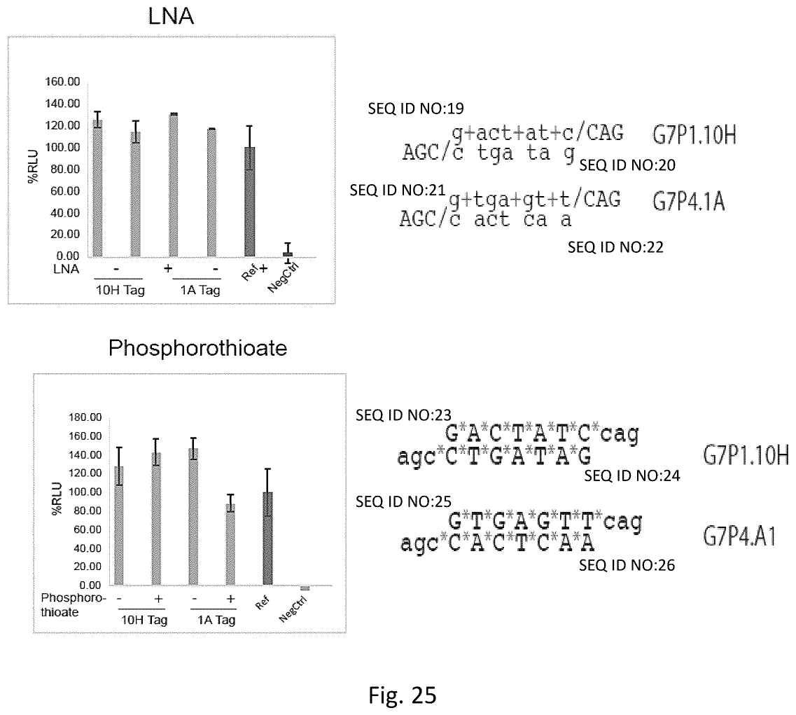

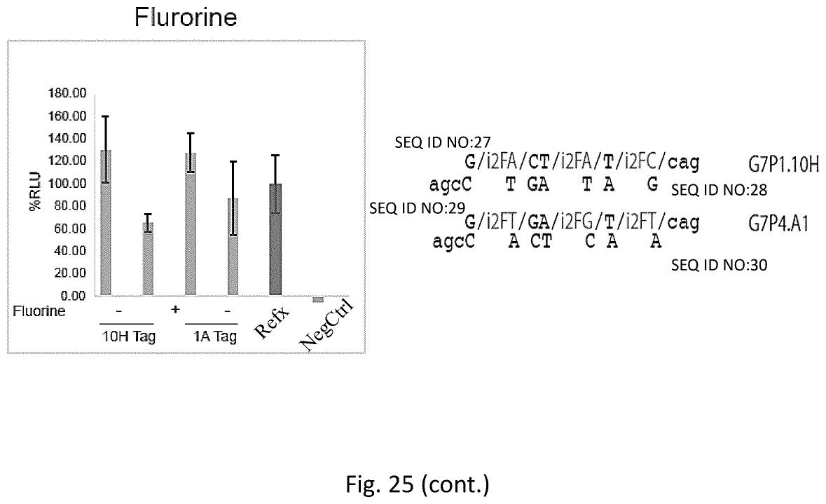

Using the DEL-ligation-kit (see Example 1), it is demonstrated that several non-natural DNA versions can be used for LNA (locked nucleic acid), phosphorothioate, and fluorine-based DNA. Importantly not all non-natural DNA worked for DEL. For example, Deoxyinosine-based DNA is not compatible, as poly-deoxinosine based-DNA easily lost their iosine bases (FIG. 25).

### Example 5: Method to Perform Comparative/Subtractive Screens

Classical HTS screening campaigns are typically screening 100,000 to 1,000,000 compounds. It is expensive, time consuming and quite heavy in term of logistic. For these reasons it is not practical to perform parallel screens (2 screens at the same time in parallel) and it is not part of the HTS process. A DEL screening campaign can easily test 10-100 millions of compounds at once and much more. Because of the size and simplicity of the assay, 2 or more parallel screens are totally feasible. This comes with a number of advantages: reproducibility, specificity, rapidity, facility to identify non-wanted hits, etc. 2 screening campaigns in parallel:—the same exact target and the exact same conditions; the screen is performed in duplicate to evaluate reproducibility—the same target in 2 different conditions (e.g. target concentration, target quantity, target origin, target purity, with and without cofactor, buffer condition, etc.) for example to increase the chances of success and/or learn about optimal conditions. —Testing two slightly different versions of the same targets (e.g. wild-type versus mutant—for example a kinase and a kinase-dead mutant)—to help identifying hits binding to the active site for example in the case the kinase dead. —Two isoforms of the same targets for specificity or universality purposes (e.g. two isoforms of the same protein but only one should be targeted therapeutically; at the contrary two isoforms of the same protein and both must be targeted) 3 or more parallel screens: For specificity reasons, more than 2 parallel screening campaigns might be needed. For robustness, more than 2 parallel screening campaigns might be needed. For biological reasons (e.g. more than 1 mutant), more than 2 parallel screening campaigns might be needed. The logic of performing 2 or more screening campaigns in parallel is the same.

A subtractive approach between 2 or more screening campaigns allows identification of compound hits that are specific to one or a given number of screen(s) only. A Venn diagram approach for 2 or more screening campaigns prioritizes candidates by allowing identification of compound hits that are common to 2 or more targets (e.g. for duplicate screens, only hits found in both screens are considered or prioritized) or, in contrast, specific to 1 of the targets only.

The subtractive approach may allow for:
a) Replication of the same screening campaign two or more times for robustness.
b) Comparing a wild-type therapeutic target to a mutant of the same target (e.g. mutation in the active site). In the case of a mutant designed to be deleterious in its active site, a subtractive screen can be utilized to distinguish compounds that bind selectively to the active site (found with WT but not with the mutant version). In that case: WT compounds−Mut compounds=compounds of interest. This approach speeds up the identification of relevant compounds, as all the compounds binding to both versions of the targets are immediately discarded as non-essential binders.
c) Comparing wild-type target to a mutant target or fusion protein (e.g. disease). A similar approach can be taken to identify compounds that bind a mutant target protein or fusion molecule but not to any of the wild-type/non-modified regions of the fusion protein.
d) Comparing targets from different species for low specificity, to identify candidate that may broadly interact with related proteins (e.g. the same target molecule of different viruses [e.g. SARS-CoV1, MERS and SARS-CoV2] if the goal is to find a drug that works for the entire family).
e) Comparing targets from different species for high specificity. Many protein targets such as G-protein coupled receptors (GPCRs), proteases, kinases, come with a family of closely related isoforms. Often, high specificity is needed not to target the entire family. Therefore a subtractive screen can be used to eliminate potential candidates that are cross reactive to other family members of a target molecule.

### Example 6: Modification of DNA to Increase Solubility in Non-Polar Solvents (e.g. Organic Solvents), and to Perform Entirely Different Chemical Reactions

The major limitation perhaps for the DEL technology is that the chemical reactions used to generate the active compounds are happening in presence of DNA. This has two direct consequences:—due to the solubility of DNA, the reactions have to happen in aqueous solution because of the polar nature of DNA. Due to the fragility of the DNA, the chemical conditions have to be mild.

Organic chemistry typically requires harsh conditions and non-aqueous solvents (also known as organic solvents). These limitations greatly reduce the possibilities of chemical reactions that are DNA-compatible, therefore limiting greatly the chemical space covered by DEL chemistry. Identifying a way to reduce DNA polarity will increase the solubility of DNA in organic solvents and decrease the need to work in aqueous solutions. This will have enormous applications in term of the type of chemical reactions that can be performed with DNA, in the context of DEL or for any other technology requiring organic chemistry in presence of DNA.

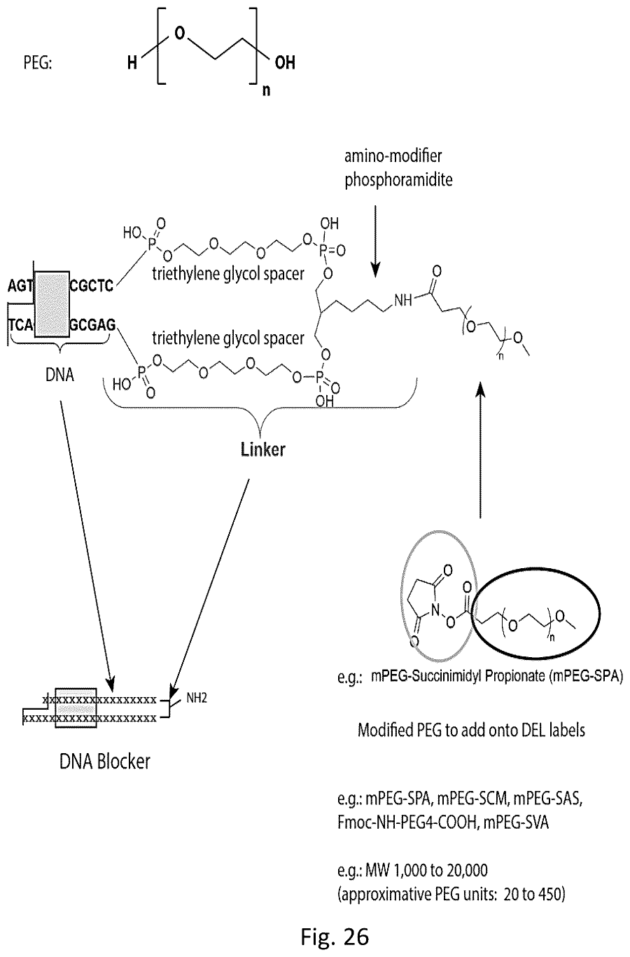

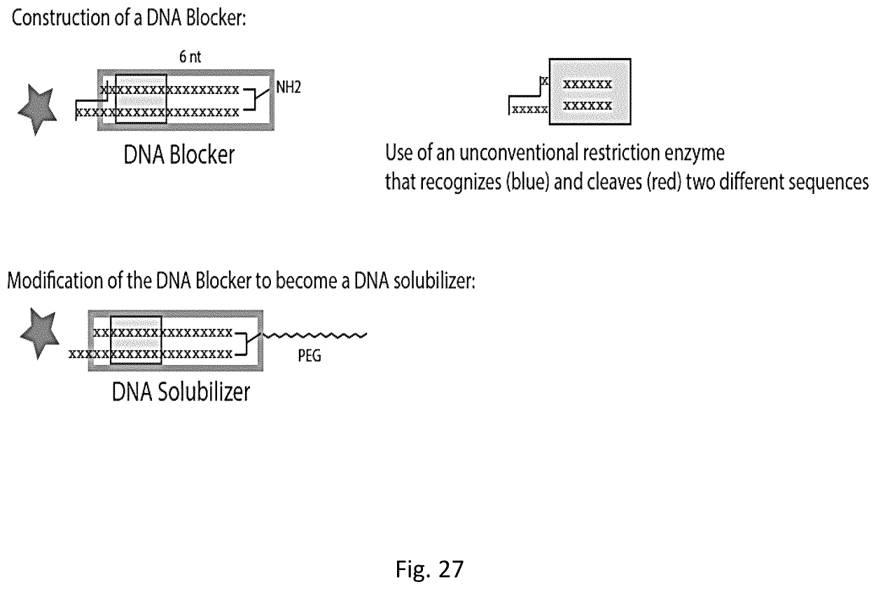

Solubility of nucleic acid molecules is increased in organic solvents by neutralizing negative charges to reduce polarity leading to a reduced hydrophilicity. This can be accomplished by attaching a removable/cleavable less-polar moiety to the nucleic acid molecule. For example polyethers (also called polyether glycols or polyols) will qualify [e.g. PEG (polyethylene glycol)]. This considerably increases the possibilities in term of chemical synthesis and significantly increases the chemical space that can be covered by DEL. A schematic is presented (FIG. 26-FIG. 29), and some exemplary of PEG molecules have been tested: mPEG-SPA, mPEG-SCM, mPEG-SAS, Fmoc-NH-PEG4-COOH, mPEG-SVA.

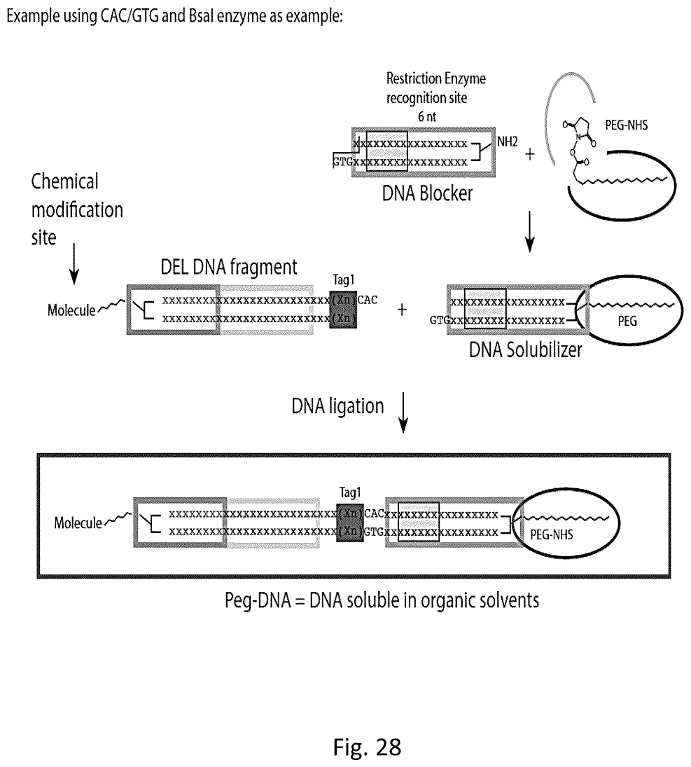

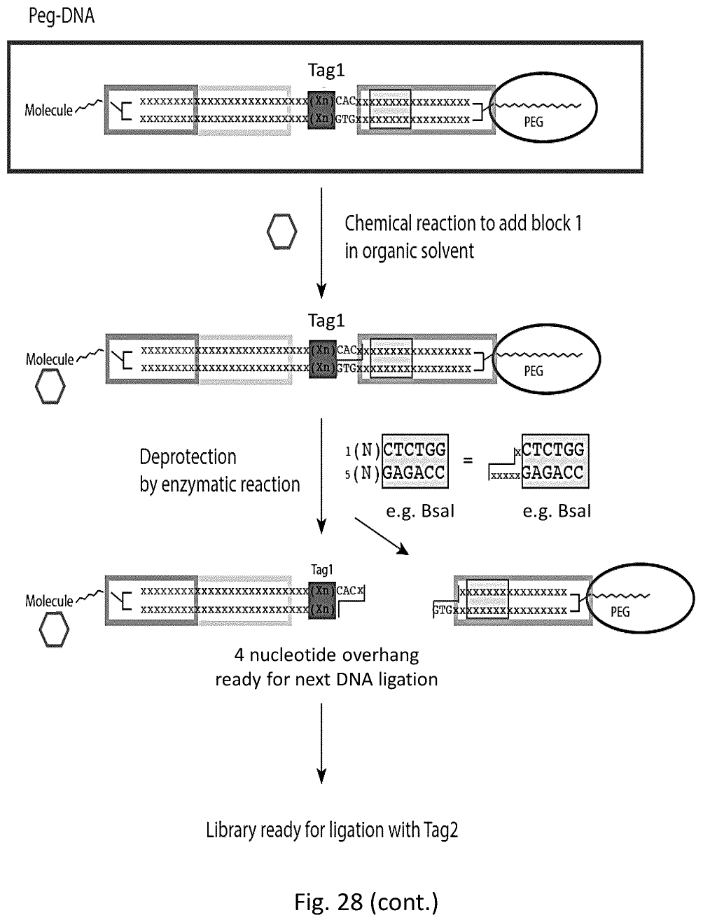

The principle is similar to those described above except that it happens during the synthesis of the DEL library. In this case the DNA fragment to be added reversibly is labeled as the “DNA blocker”. This DNA fragment has a free chemical group such as a free functional amine onto which a functionalized PEG molecule is added as shown (FIG. 28—blue circle is the function to react with free amine and purple circle indicates the PEG molecule). Once the PEG is added to the DNA blocker, the new fragment is termed a “DNA solubilizer”.

A special restriction site using an unconventional restriction enzyme is incorporated (see blue rectangle). The enzyme chosen (e.g. Bsa1) recognizes 6 nucleotides and cut outside of this recognition sequence in position +1/+5. By creating the overhang to be compatible with the DEL label being made (e.g. after tag1 in our example), we will ligate this DNA stabilizer instead of Tag2 for example (as usually with 3 overhang nucleotides). Now that the DEL labels are ligated to the DNA stabilizer fragment, the PEG-DNA can undergo chemical reaction in nonaqueous solvent. In a new series of experiments it is demonstrated that different sizes of PEG (MW 1,000 to MW 20,000) effectively alter DNA solubility and increase DNA solubility in organic solvents. It is demonstrated that this works with 6 different organic solvents of high to low polarity (e.g., DMSO; DMA; DMF; 1,4-Dioxane; DCM; ACN). Furthermore the data demonstrates that with 1 chemical reaction and 1 chemical block that the DNA piece linked to a DNA solubilizer can effectively be modified in an organic solvent (the chemical reaction used does not work in water). Once done, the DNA solubilizer is removed with the unconventional enzyme (e.g. BsaI) that cuts away from its restriction site, recreating the overhang after Tag1, but this time with 4 and not 3 single strand nucleotides. This offers the possibility to reverse the addition of the DNA solubilizer and at the end by adding only 1 extra nucleotide—this extra nucleotide could be used actually to distinguish between the labels that did not undergo the process (still 3 overhang nucleotides) from the ones that actually did and that now have 4 overhang nucleotides.

The solvents/reactions that are DNA compatible after adding a PEG or any other solubilizing moiety including, but not limited to DMSO; DMA; DMF; 1,4-Dioxane; DCM; and ACN. Polyethers (also called polyether glycols or polyols), PEG being one such example, will change DNA properties as needed. More broadly any moiety that can neutralize and/or reduce DNA polarity will help reducing hydrophilicity and therefore increase solubility in organic solvents.

### Example 7: SELDA, a Sandwich Enzyme-Linked DNA-Ligation Assay Relevance for DNA-Encoded Library (DEL) Technology

The grounded premise of the DEL technology is rooted in the use of short DNA sequences that are used to barcode organic compounds in order to be able to identify them later by decoding the DNA sequence covalently linked to them. Therefore, the DNA tagging system is extremely important and dictates the overall quality of a DEL platform, starting with the interpretation of screening campaign results. As good and as unique as the chemistry of a given DEL library can be, if the DNA barcoding is not perfectly adjusted, the noise will be high and great chemical compounds might not be retrieved. Considering that a DEL DNA label is created through the successive addition of several DNA fragments using a DNA ligase, the highest ligation efficiency is necessary to ensure the proper labelling of the associated chemical compounds. The experiments detailed below demonstrate a trustworthy quantitative and miniaturized method (SELDA for sandwich enzyme linked DNA-ligation assay) to measure and calibrate DNA tag ligation efficiency. This method is also useful to measure and evaluate the impact of a large number of parameters and treatments on the integrity of DNA fragments.

SELDA was used to prove that DNA tag design can affect the DNA tag's ability to ligate efficiently. A number of parameters have been investigated in the present study: the overhang length, the tag nucleotide length, the percentage of GC content, the dependence of 5′-phosphate, the asymmetrical overhang length, and the presence of non-natural nucleotides in the tag sequence. Convincingly, the results obtained showed that increasing the number of overhang nucleotides starting from 2, as well as the length of the DNA tag sequence, did not alter at all the ligation efficiency. Working with longer overhangs and DNA tag sequences can confer the advantage of a more stable structure and prevent disassociation of the pre-ligation fragments and de-annealing. As expected, neither the percentage of GC/AT content in the tag sequence (excluding the OH), nor the asymmetrical overhang length, did affect the ligation efficiency. The ligation remained fully efficient as long as the percentage of 5′-phosphate was 40% or higher. Indeed, one of the DNA modifications that is observed due to the conditions of some chemical reactions necessary during the construction of a DEL library is the loss of the 5′-phosphate. While this is an important parameter, it is known that the ligation of a DNA fragment requires a 5′-phosphate group to be present on each strand, this parameter demonstrated some level of flexibility in the way SELDA was calibrated in the present study. If phosphorylation was to be the main concern (e.g., for testing DNA fragments following chemical reactions) the SELDA assay could be easily calibrated to not allow flexibility on the 5′-phosphate group, by adjusting tag concentration to be limiting.

Interestingly, incorporating non-natural nucleotides such as LNA, phosphorothioate- or fluorine-coupled nucleotides, within the DNA tag sequence, did not affect the ligation efficiency. LNA and phosphorothioate-coupled nucleotides are both more resistant to nuclease degradation thus conferring an advantage in term of stability. Incorporating fluorine-coupled nucleotides can add value to a given compound for tracing purposes for example. Surprisingly, the presence of deoxyinosines nucleotides totally abolished ligation even though they are known to form base pairs with conventional natural bases. However, it has been demonstrated that the Deoxyinosine-Cytosine pair was less stable than the Adenine-Thymine pair (Martin et al., Nucleic Acids Res. 1985; 13(24):8927-38). Thus, it cannot be excluded that the deoxyinosine base-pairing is less stable in the conditions presented here, especially considering the small size of the DNA fragments used. The absence of ligation most likely indicates that the very short DNA fragments containing deoxyinosine are not annealed.

In summary, all the different designed tags underwent efficient ligation except when the overhang length was smaller than 2 nucleotides, when the 5′-phosphate percentage was below 40%, or when the nucleotides were replaced by the deoxyinosines. Those results further demonstrated the versatility of SELDA that allows to take into account all the parameters of a DNA fragment to be ligated and that are almost impossible to verify, visualize or quantify, by other methods except partially perhaps by LCMS. Each parameter has been tested individually here, but it is also known that the cumulative impact of a slight deficiency of several of those parameters is synergistic and leads quickly to a total ligation inhibition.

In the context of DEL more specifically, SELDA allows DNA fragments to be reliably tested and standardized for what matters the most, their capacity to be added to another DNA fragment by DNA ligation, prior to being incorporated into a DEL library. For example, out of 60 different tag sequences tested and designed for a three split-and-pool steps DEL library, 12 tags showed lower ligation efficiency (below 75%) and 1 tag showed no ligation. Surprisingly, all defective tags were found in one of the three series designed. The impact of different overhang sequences at the ligation level can here be questioned. Indeed, the sequence of the 3′ overhangs of the Tag #1 series could have been less stable after ligation than the overhang sequences of the two other Tag #series. By testing this set of tags in a high-throughput manner, its intrinsic variability was demonstrated. SELDA offers a systematic method to not only correct this intrinsic variability but also to identify DNA tags that would have to be discarded because of poor or null ligation efficiency. It is not clear why one of the 60 tags failed to ligate (nature of the sequence or quality of the DNA preparation/synthesis) but it is clear that this tag was non-usable. Indeed, a number of repeats were performed and different parameters (e.g., tag concentration, temperature and duration of ligation) were tested to ensure that its ligation efficiency could not be enhanced. None of these conditions led to any ligation signal confirming that something was wrong with this tag. Importantly, the incorporation of this tag in first position of a three-steps DEL library (20×20×20) would have caused for 400 molecules out of 8,000 not to be represented. Each defective tag1 could lead to 10,000 not to be represented at all in a 100×100×100 DEL library or even 100,000 molecules in a 1,000×1,000×1,000 DEL library. In other words, for every 1% of defective tags in Step1, it is up to 10 million molecules that can be absent in the case of a 1,000×1,000×1,000 DEL library.

Another advantage of the SELDA assay is the possibility of checking the proportion of non-ligated tags in the mixture after DNA ligation. One can then optimize the DNA tags concentration in the ligation assay to aim at minimizing the presence of non-ligated tags.

Furthermore, SELDA represents a quick and scalable solution to test, in a quantitative manner, the impact of chemical conditions on the integrity of DNA, on DNA ligation. This is especially relevant to evaluate novel chemical reaction conditions. It will greatly help explore and increase the chemical space accessible to DNA-compatible reactions that are essential for the DEL field to grow.

The SELDA assay can also be indispensable for evaluating the quality of DNA fragments after being exposed to various treatments and conditions such as the ones imposed when performing organic chemistry reactions, but not only. The construction of a DEL library indeed requires the heavy intervention of organic chemistry and it is therefore crucial to assess the impact of experimental conditions on DNA ligation efficiency. Different conditions were used as example and dramatic differences in term of DNA ligation were showed when changing the pH by just one unit, or by increasing the incubation time. This clearly demonstrate the usefulness of SELDA to evaluate functionally the impacts of chemical conditions onto DNA. SELDA could be used virtually to test repercussions that any chemical condition might have on DNA. Some differences between the series of DNA tags were totally unexpected and it demonstrates fully the need to systematically test all DNA tags and conditions, without trying to predict or accept apparent similarities. Surprisingly, the Tag #2 did not show any alteration in term of ligation. One difference observed for the Tag #2 comparing to the Tag #1 and Tag #3 series was the nature of the last nucleotide located at the overhang: both Tag #2 overhangs terminated with a Guanine while the two overhangs of Tag #1 and Tag #3 were composed by Adenine-Guanine and Thymine-Guanine respectively. Moreover, a study describing the base-phosphate interaction stability of each RNA nucleotide revealed that Guanine possessed the most stable base-phosphate interaction comparing to the other bases (10). Therefore, without being bound by theory, one hypothesis to explain why the ligation of Tag #2 was not impacted was that the 5′-phosphate necessary for the ligation, was more resistant to harsh treatment when linked to Guanine than to Adenine or Thymine.

The results presented in this study confirm that after 2 years at −80° C. in TE (pH 8.0) buffer almost all the tags tested remained totally competent in term of ligation efficiency except for one tag (2.6) for which the SELDA signal dropped below the quality threshold of 75% in comparison to the value obtained 2 years prior. If a DEL library was to be made at this point, the 2.6 tag should not be included anymore due to its poor ligation efficiency. Altogether, these results demonstrated the importance of monitoring the DNA quality over time, which became easily accessible with the SELDA assay.

So far, techniques allowing the monitoring of a ligated DNA product were limited to the non-quantitative use of DNA electrophoresis on agarose gel or to non-throughput and expensive methods such as BioAnalyzer. These methods are typically not appropriate for DNA fragments of smaller size. In addition, none was suitable for a high throughput analysis. The SELDA assay developed here represents the first high throughput system to measure precisely and quantitatively the ligation efficiency of any fragment of DNA. We have demonstrated that SELDA worked with a plethora of DNA sequences that could differ in term of length, nucleotide nature, nucleotide content, etc. For DEL purposes, SELDA became an important tool to verify the ligation efficiency of the DNA tags designated to be used in a DEL construction to ensure the correct labelling of the associated chemical compound, hence the quality of the final library. Last but not least, with SELDA, it became possible to evaluate the degree of DNA degradation acceptable for DNA ligation, as a result of specific conditions ranging from long term-storage to chemical treatments. In summary, SELDA is a reliable technique that allows quantitative measures of DNA ligation efficiency in a high throughput manner.

The experimental materials and methods are described.

### DNA Fragments

The DNA fragments were synthetized by IDT (Coralville, Iowa, USA) and resuspended in T.E. pH 7.4 upon arrival at 100 μM. All DNA fragments were kept at 80° C. Unless specified, the DNA fragments were designed to contain 25-75% GC, no hairpin, and no stretches of 3 or more G or C nucleotides.

The first set of DNA tags used with SELDA was intended to study the impact of different tag designs on the ligation efficiency: overhang length (1 to 5 nt), overall length (5 to 25 nt), the percentage of Guanine and Cytosine content (0 to 100%), the percentage of the 5′ phosphate (100 to 0%), the asymmetrical overhang length (3-3 nt versus 2-4 nt), and the use of non-natural nucleotides. Each experiment has been analyzed in duplicate and the corresponding tags were named Tag #x.1 and Tag #x.2 to distinguish two designs.

The second set of DNA tags was used as a proof of concept of the use of SELDA in a high throughput manner. They corresponded to a total of 60 tags, split in 3 different series hereafter called Tag #1, Tag #2 and Tag #3 series (Table 1). Each series contained 20 tags.

All the studied tags have been designed manually and checked for their unicity, non-palindromic sequences, the absence of hairpin structures. The OH ends have been designed to have all 1 A/T nucleotide and 2 G/C nucleotides.

TABLE 1
Series of tags
Tag Series #1 (positive strands shown)

| | | | |
|---|---|---|---|
| TCGGAGAA | TCGGAGCT | TCGGATAC | TCGGACGT |
| TCGGAGAT | TCGGAAGA | TCGGATTG | TCGGACAA |
| TCGGAGTA | TCGGAAGT | TCGGATCA | TCGGACAT |
| TCGGAGTT | TCGGAACA | TCGGATCT | TCGGACTA |
| TCGGAGCA | TCGGAACT | TCGGACGA | TCGGACTT |

Tag Series #2 (positive strands shown)

| | | | |
|---|---|---|---|
| CAGAGGAG | CAGAGGTA | CAGAGAGG | CAGAGAAC |
| CAGAGGAA | CAGAGGTT | CAGAGAGA | CAGAGATG |
| CAGAGGAT | CAGAGGTC | CAGAGAGT | CAGAGATC |
| CAGAGGAC | CAGAGGCA | CAGAGAGC | CAGAGACG |
| CAGAGGTG | CAGAGGCT | CAGAGAAG | CAGAGACA |

Tag Series #3 (positive strands shown)

| | | | |
|---|---|---|---|
| ACGTGGAG | ACGTGGTA | ACGTGAGG | ACGTGAAC |
| ACGTGGAA | ACGTGGTT | ACGTGAGA | ACGTGATG |
| ACGTGGAT | ACGTGGTC | ACGTGAGT | ACGTGATC |
| ACGTGGAC | ACGTGGCA | ACGTGAGC | ACGTGACG |
| ACGTGGTG | ACGTGGCT | ACGTGAAG | ACGTGACA |

### Ligation Procedure

All DNA ligations were performed using 1 μM of the DNA tag of interest and 1 μM of the corresponding complementary f-Biotin and f-DIG oligonucleotides, in a presence of 500 units of T4 DNA ligase (New England Biolabs, Rowley, MA, USA) in 1× ligase buffer. The mixture was incubated 1 hour at 16° C., unless otherwise specified, and immediately used in the SELDA assay at the appropriate dilution.

### Sandwich Enzyme-Linked DNA-Ligation Assay (SELDA)

Opaque white 96-well plates coated with streptavidin (#15218, Thermo Fisher, USA) were used. Prior use, the plates were washed 3 times 5 min with 150 μl of washing buffer (25 mM Tris, 150 mM NaCl pH 7.2; 0.1% BSA; 0.05% Tween-20). The DNA ligated products/ligation mixtures to be tested were carried out in tubes and diluted prior adding it into the wells (1 nM) in a total volume of 100 μl. The reaction was allowed to incubate for 2 hours at room temperature. The wells were then washed 3 times 5 min with 150 μl of washing buffer before adding 200 μl (dilution 10⁶) of an anti-DIGHRP antibody (#NEF832001EA, Perkin Elmer, US). After 30 min of antibody incubation at 5 room temperature, the wells were washed 3 times 5 min. Finally, 150 μl of HRP substrate (#37075, Thermo Fisher, US) was added per well and after 5 min the luminescence signal was measured at 425 nm using an Envision 2104 Multimode Plate Reader (PerkinElmer, NY, USA). The values are expressed as Relative Light Unit (RLU). Biotin (#B4501, SIGMA, USA) and DIG-NHS Ester (#55865, SIGMA, US) were also tested alone to ensure that they did not interfere with the signal obtained.

### TBE 20% Gel Electrophoresis

The DNA ligation products to be analyzed by gel electrophoresis were loaded on acrylamide gels (TBE 20%)(#EC6315BOX, Thermo Fisher, US) and ran at 30 V for 30 min. TBE gels were then incubated in a 0.0001% ethidium bromide/H2O solution for 20 min before being rinsed in ultrapure H₂O for 20 min two times. The gels were scanned under a 302 nm UV light in an Azure Biosystems C200 apparatus (Azure Biosystems, CA, USA).

### Statistics/Duplications/Repeats

All studies were conducted in triplicate (individual experiments) and the data points for each were performed in duplicates. Comparison of two groups was statistically analyzed using a two-tailed t-test. Error bars indicated in the figures represent the standard error of the mean (S.E.M.).

The experimental results are now described.

### SELDA Principle

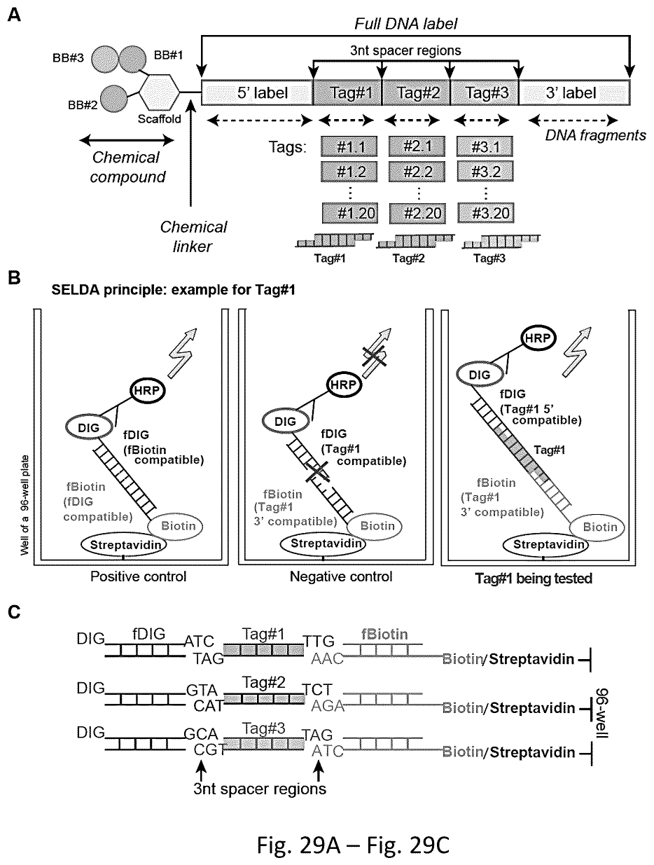

The initial purpose of creating a SELDA assay (FIG. 29B) is to provide quick, high throughput, quantitative and functional information about DNA ligation efficiency of a large set of small DNA fragments that are not reliably quantifiable and for which the ligation capacity is the only crucial parameter. The method is based on the ability of the DNA fragment of interest (e.g., DEL DNA tag; here Tag #1, 2 or 3) to be sandwiched for DNA ligation (3-way ligation) between two different accessory DNA fragments (f), a DNA fragment (e.g., fBiotin) that is covalently immobilized in a well (e.g., streptavidin interaction) and a second accessory DNA fragment (e.g., fDIG), enzyme-linked, that harbors a DIG moiety and can be coupled to HRP via an anti-DIG antibody covalently linked to HRP (horse radish peroxidase)(anti-DIGHRP antibody). The luminescence signal generated when all the pieces are assembled successfully is proportional to the ligation efficiency of the intermediary DNA fragment to be tested, on both ends at once.

Blunt-end ligations do not allow for directional DNA ligations. Therefore, they are not useful to build a DEL platform and consequently only cohesive-end ligations will be used in this study. However, quantification by SELDA of blunt-end ligations would work just as well. In general, the present study focusses on DEL applications and a number of experimental choices were made based on the direct relevance for constructing DEL libraries.

### SELDA Assay

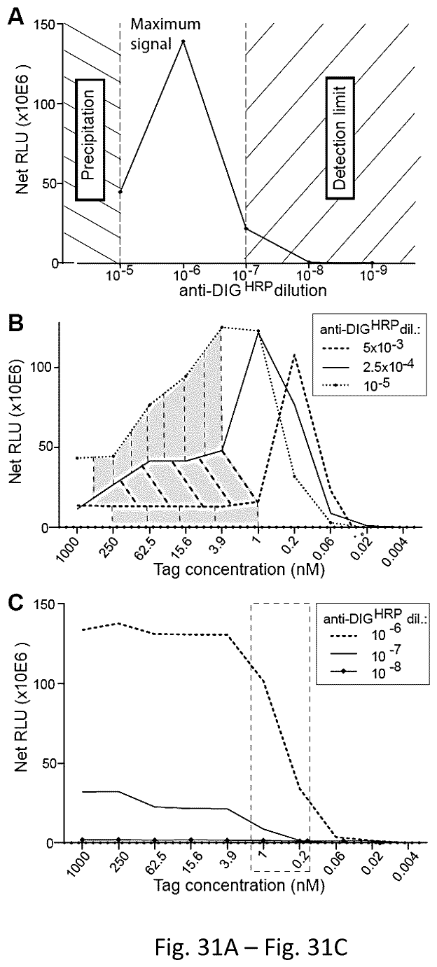

The maximal HRP intensity signal that can be obtained from a 96-well format coated with streptavidin in function of the ligation volume was estimated using a 2-way ligation assay (FIG. 29C) in which no test fragment was used. In this configuration, fBiotin and fDIG harbors complementary cohesive ends. First, a range of increasing dilutions of anti-DIGHRP antibody was tested using a fix total volume (FIG. 31A). Chemiluminescence was measured at 450 nm after 5 minutes of incubation in the presence of 200 μl of HRP substrate and no washes were performed. The maximum signal in these conditions was 140×10⁶ RLU when the anti-DIGHRP was used at a 10⁻⁶ dilution (hereafter called maximum signal) (FIG. 31A). A dilution factor of the antibody greater than 10⁷ led to noise signal, while a high concentration of the antibody (dilution factor of 105 or lower) caused a drop of the luminescence signal, most likely due to the antibody precipitating. A counter assay in which fBiotin and fDIG harbors non-complementary cohesive ends (FIG. 29B) was used to define the background signal. For future experiments, these two SELDA configurations will represent the positive and negative controls.

The DNA fragment to be tested (DNA-2bT) harbors a 3′OH overhang sequence on each strand. To take into account for the directionality of the ligation, the two 3′ ends must be different. The ligation occurs between DNA-2bT and fBiotin on the positive strand and between DNA-2bT and fDIG on the negative strand (FIG. 29B). The T4 DNA ligation between the three DNA fragments is carried out simultaneously in a well of a 96 well plate and an aliquot of the ligation mixture is transferred and diluted before being added to a streptavidin coated well. If the ligation occurred successfully, the DNA-2bT is then immobilized onto the streptavidin-coated well through the strong streptavidin-biotin interaction which results in HRP coupling. In summary only DNA-2bT ligated on both ends are retained and can participate in luminescence production (FIG. 29B).

### SELDA Optimization

First, DNA-2bT optimal concentration had to be determined. The most crucial component was to uniformize the assay by comparing and normalizing ligation efficiency between all DNA fragments to be used. Therefore, it was important to evaluate the signal dynamic range. Concentrations of fDIG/fBIOTIN ranging from 1 μM to 4 μM for the positive control (see schematic FIG. 29B) were tested and six anti-DIGHRP antibody dilutions were tested (5·10 3, 2.5·10⁻⁴, 10⁻⁵ in FIG. 31B, and 10⁶, 10⁷, 10⁸ in FIG. 31C). The signal dynamic range for the first three anti-DIGHRP dilutions was as follows: DNA-2bT 0.2 to 0.8 nM, 0.08 to 0.5 nM, and 0.06 to 0.1 nM respectively (FIG. 31B). For the three highest tag concentrations, the signal appeared not to be stable and dropped hastily, most likely to anti-DIGHRP precipitation (non-stable signal range highlighted with grey dashed lines)(FIG. 31B). None of the remaining anti-DIGHRP concentrations tested (dilutions 10⁶, 10⁷, and 10⁸)(FIG. 31C) caused antibody precipitation even at the highest DNA concentrations used. As a result, in this volume range, SELDA can be performed at DNA concentrations ranging from 0.5 nM to 2 nM (FIG. 31C, dashed line rectangle) using the anti-DIGHRP highly diluted 10⁶ times to allow dynamic comparison of ligation efficiencies between the samples to be tested.

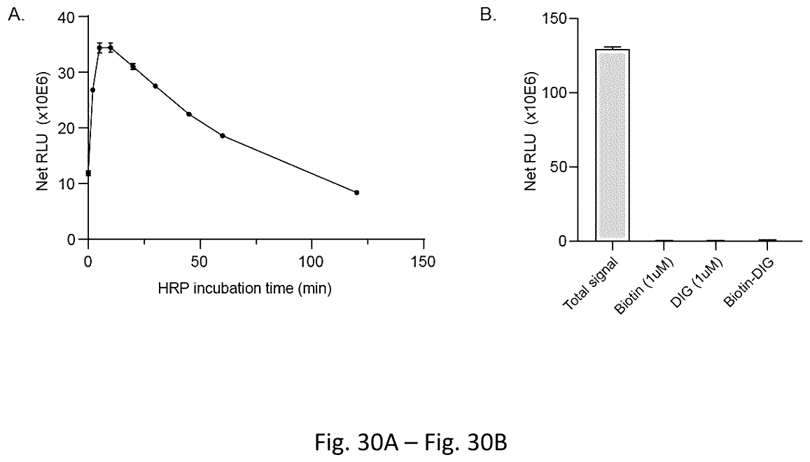

Next, the stability of the luminescent signal was investigated by performing a time course experiment (0, 2, 5, 10, 20, 30, 45, 60 and 120 minutes) and measuring the HRP signal (dilution 10⁶). The signal measured reached the maximum after 5 minutes and remained stable for up to 10 minutes (FIG. 30A). After this time period, the signal decreased significantly. For future experiments, the luminescence measurement will be assessed between 5 and 10 minutes after the HRP substrate incubation is initiated. Finally, several other negative controls were assayed. As expected, Biotin, DIG-NHS Ester, as well as a mix of Biotin and DIG, did not produce any luminescent signal compared to the total signal (FIG. 30B). All together, these results clearly demonstrate that the luminescence read out is specific to the ligated product.

### Versatility of SELDA

Using the optimal conditions previously defined, different DNA fragments parameters were tested to evaluate the level of flexibility of SELDA. For each property tested, two different DNA fragments called Tag #A and Tag #B were used (FIG. 29A); they share the same parameters (length, concentration, purity, cohesive ends) at the exception of their nucleotide sequence.

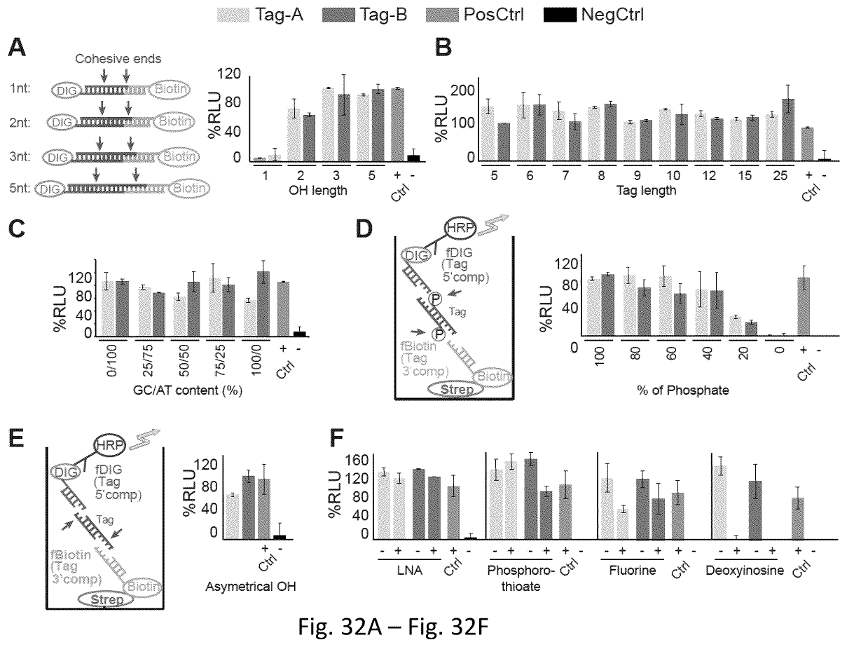

First, to evaluate whether the overhang (OH) length affects the ligation efficiency in the conditions used, DNA fragments with 1, 2, 3 or 5 OH nucleotides (nt) were generated, subjected to ligation and tested by SELDA. Tag-A and Tag-B showed almost optimal ligation efficiency for 2 nt-OH (73% and 63% RLU respectively) and a perfect ligation efficiency for 3 nt-OH (101% and 92% RLU respectively) and 5 nt-OH (91% and 96% RLU respectively); the signal from the positive control represents 100% RLU. In these conditions, 1 nt-OH ligation showed a quasi-null ligation efficiency (5% RLU) and was equal to noise given that the negative control was slightly higher (8.4% RLU) (FIG. 32A).

Next, DNA fragments of various lengths (5- to 10-, 12-, 15- or 25-nucleotides long) were evaluated. The positive control signal value was set at 100% RLU; the negative control signal value was 7% RLU. All DNA Tag versions underwent successful ligations. For Tag-A and Tag-B respectively, expressed in % RLU, efficiencies were as follows: 5 nt, 165% and 116%; 6 nt, 167% and 171%; 7 nt, 151% and 120%; 8 nt, 161% and 173%; 9 nt, 117% and 124%; 10 nt, 155% and 142%; 12 nt, 142% and 129%; 15 nt, 125% and 133%; 25 nt, 140% and 189% (FIG. 32B).

Next, the impact of the DNA fragment composition (GC versus AT nucleotides) was assessed. Different versions of Tag #A and Tag #B with 0, 25, 50, 75 or 100% of GC nucleotides were evaluated. The positive control signal value was set at 100% RLU; the negative control signal value was 8% RLU. All DNA Tag versions underwent successful ligations. For Tag-A and Tag-B respectively, expressed in % RLU, efficiencies were as follows: 0% GC, 100% and 98%; 25% GC, 90% and 78%; 50% GC, 73% and 98%; 75% GC, 106% and 93%; 100% GC, 66% and 115% (FIG. 32C).

DNA ligation requires the presence of a 5′-phosphate group on each strand to allow for covalent bonding. To investigate the impact of phosphorylation by SELDA, two identical DNA fragments, one phosphorylated, and one non-phosphorylated, were mixed at different ratios to achieve different levels of phosphorylation (100%, 80%, 60%, 40%, 20% and 0%). Similarly, to the positive control (100% RLU), the ligation occurred for both tags tested at a phosphorylation level of 40% or higher. For Tag-A and Tag-B respectively, expressed in RLU, efficiencies were as follows: 100% phosphate, 98% and 105% RLU; 80% phosphate, 103% and 82% RLU; 60% phosphate, 102% and 73% RLU; 40% phosphate, 80% and 78% RLU). As expected, the ligation efficiency decreased significantly when the phosphorylation level reached 20% (33% and 24% RLU) and became comparable to the negative control when no phosphate was present (FIG. 32D).

Cohesive-end DNA ligations are typically involving one stretch of single nucleotides of the same size on each side of the DNA fragment to be ligated. To demonstrate the flexibility of SELDA, and to anticipate future applications, an asymmetrical ligation was tested using two DNA fragments harboring 4 and 2 overhang nucleotides. Results revealed that the asymmetrical OH length did not affect the ligation efficiency at all (74% and 105% RLU). The positive control showed efficient ligation (100% RLU), while no ligation was observed for the negative control (6% RLU) (FIG. 32E).

The last modified DNA fragment parameter investigated was the nature of the nucleotides themselves. To evaluate whether incorporating non-natural nucleotides such as “locked nucleic acids” (LNA), deoxyinosines or nucleotides coupled with phosphorothioate or fluorine, could impact on the ligation efficiency, a series of experiments were performed testing each where the natural DNA fragment (−) was compared with the modified fragments harboring the modifications (+). Remarkably, replacing half of the natural nucleotides in a given DNA fragment with LNA nucleotides gave comparable ligation efficiency as the natural DNA fragment (126% and 131% RLU for DNA versus 115% and 118% RLU for LNA). The ligation was also as efficient in the presence of phosphorothioate-coupled nucleotides (128% and 147% RLU for DNA versus 143% and 88% RLU for phosphorothioate fragments). A slight decrease in the ligation efficiency average was observed for the tags containing fluorine-coupled nucleotides compared to natural DNA (131% and 128% RLU versus 65% and 87% for the modified nucleotides). However, the efficiency measured for both tags harboring fluorine still reflected a significant ligation efficiency when compared to the positive control (100% RLU). Surprisingly, the replacement of half of the natural nucleotides by deoxyinosines abolished the ligation. Indeed, no signal was detected for both of the DNA fragments containing deoxyinosines tested. Systematically, for all conditions tested, the positive controls showed 100% RLU while close to no signal could be measured for the negative controls (FIG. 32F).

### Variability of Ligation Efficiency Among 60 DNA Fragments

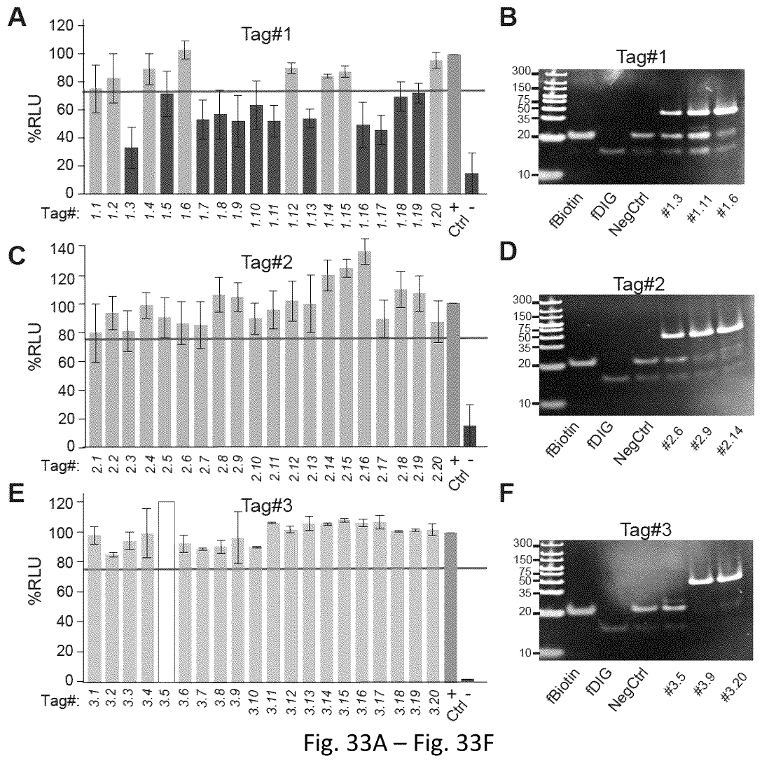

Split-and-pool DEL libraries are often made of 3 or 4 successive rounds of split-and-pool steps. In order to follow this logic, and as a proof of concept that SELDA can be used in a high-throughput manner, 60 DNA fragments corresponding to 3 sets of tags (Tag #1 [pink], Tag #2 [purple] and Tag #3 [blue]) were designed. Each set had different 3′-OH cohesive ends and can be used for 1 step of a 3 split-and-pool step DEL and a variant of fBIOTIN and fDIG was designed for each Tag #series. The design for each of the 60 DNA tags sequences were as described previously and based on successful ligations: coding tags 7 nucleotide-long, symmetrical overhangs of 3 nucleotides, 50% GC content, 100% 5′-phosphate and all-natural nucleotides. Positive controls were set at 100% RLU. Each tag has been analyzed in duplicate and each of the 3 series was tested in triplicate. Dramatic differences were observed between the 60 tags ranging from 0% ligation to 124.55% of our control signal. Importantly, one DNA tag (#3.5) showing no ligation signal was easily identified by SELDA; this DNA tag must be discarded and cannot be utilized in the construction of a DEL library. A signal equal to 75% of the positive control or greater was considered acceptable and the corresponding tags can be included in the construction of a DEL library. Based on this 75% threshold, 60% of the tags belonging to the Tag #1 series were not consider as efficient tags in term of ligation (represented in red) (FIG. 33A, FIG. 33C and FIG. 33E).

### Comparison of SELDA and Gel Electrophoresis Analysis

Three ligation reactions of the Tag #1 series and corresponding to three different levels of ligation efficiency by SELDA (tags #1.3 [33% RLU], #1.11 [52% RLU] and #1.6 [103% RLU]) were analyzed by agarose gel electrophoresis. Three ligation products of tags that showed a luminescent signal lower than 75% RLU, their corresponding fBiotin (˜21 bp) and fDIG (˜16 bp) fragments, as well as the negative control were analyzed by agarose gel electrophoresis. All fragments and the ligated product (˜50 bp) were clearly separated and visible. It is quite clear that Tag #1.3 led to significantly less ligated product. For Tag #1.11, the gel results are indicating that significant amounts of non-ligated fragments remain (FIG. 33B). Based on only this gel analysis and the ligated products, all three tags would have been probably used in a DEL library while SELDA is indicating that only one of them is acceptable. These results demonstrate that the tag ligation efficiency obtained by gel electrophoresis correlate somehow with the results obtained from SELDA, but SELDA is quantitative and more accurate, and can be performed in a high throughput manner.

### Influence of Different Ligation Conditions on Tag Ligation Efficiency

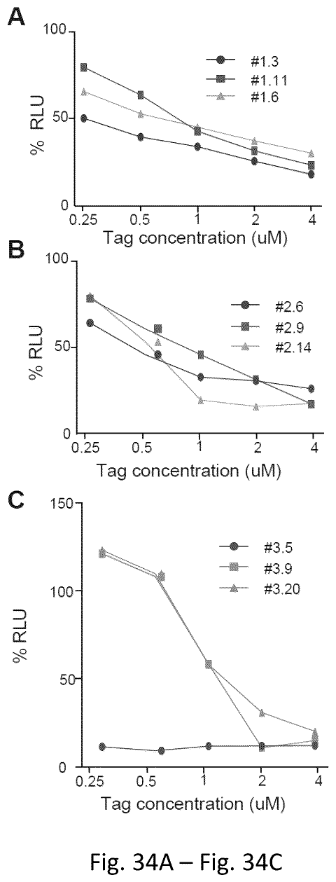

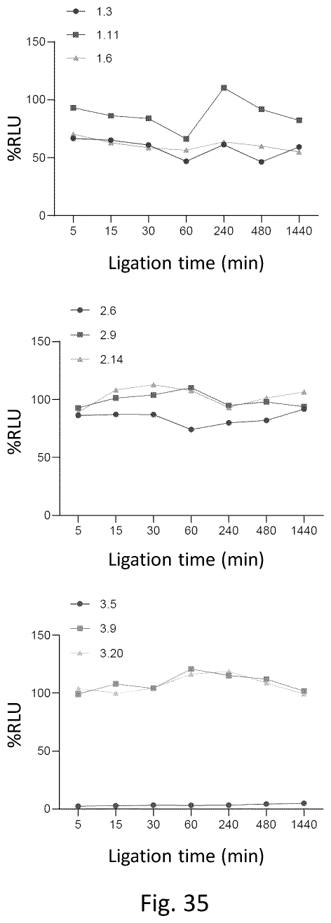

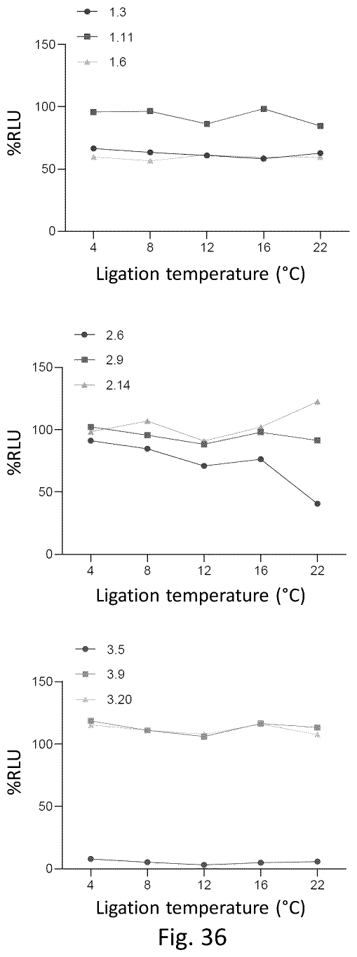

Whether the ligation parameters (tag concentration, incubation time and ligation temperature) could enhance the ligation efficiency of problematic tags was investigated. For most of them, results showed that the ligation efficiency was improved only when the tag concentration was significantly decreased (up to 16-fold). For Tag #1 series (tags #1.3, 1.11 and #1.6) SELDA values were 13%, 18% and 25% RLU for 4 μM of tags and 45%, 74% and 60% respectively for 0.25 μM of tags (FIG. 34A). Similarly, the SELDA values for tags #2.6, 2.9 and #2.14 were 33%, 24% and 25% at 4 μM and 70%, 84% and 86% at 0.25 μM (FIG. 34B). The ligation efficiency for tags #3.9 and #3.20 was 9% and 14% respectively at 4 μM and 115% and 117% at 0.25 μM. Importantly, the tag 3.5 that presented no significant ligation (6%) was not improved despite the decrease of tag concentration (FIG. 34C). No significant improvement was observed on the ligation efficiency for all the DNA tags tested, when ligation time or temperature were increased (FIG. 35 and FIG. 36).

### Impact of Long-Term Storage on DNA Ligation Efficiency

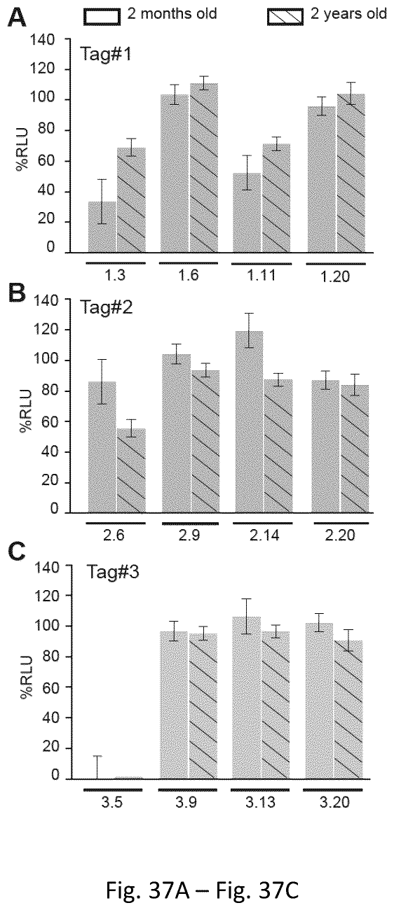

SELDA also offers the possibility to test DNA fragments that have been subjected to conditions that could potentially lead to impactful modifications and ultimately to their degradation. Considering the monetary value of DNA tags, especially once they have been tested and calibrated via SELDA, and considering that only a fraction of each tag might be used for a given library, one can wonder if DNA tags can be kept for long period of times without any deterioration. DNA tags that have been stored 2 years at −80° C. were tested by SELDA. Overall, no significant differences were observed when tags from the 3 different series were tested after two years at 80° C. For example, tags #1.6 and #1.20 showed values of 103.3% vs. 111% and 95.8% vs. 104% RLU respectively (FIG. 37A). Other DNA tags presenting different 3′ OH also showed no significant difference in ligation efficiency after 2 years. For example, tags #2.9: 104.3% vs. 93.4%; #2.14: 119.5% vs. 87.4%; #2.20: 87.1% vs. 84.1%; #3.9: 95.4% vs. 96.6%; #3.13: 96.5% vs. 106.3%; #3.20: 91% vs. 102.2% RLU respectively (FIG. 37B, FIG. 37C). Out of 60 tags tested, only 1 only (tag #2.6) which had an initial ligation efficiency of 86% RLU dropped below our quality threshold of 75% after 2 years of storage (55.5% RLU) (FIG. 37B). As expected, the DNA tag #3.5 that did not ligate initially (0.05% RLU) remained unchanged after being stored 2 years at 80° C. (FIG. 37C).

### Impact of pH and Temperature on DNA Tags Ligation Efficiency

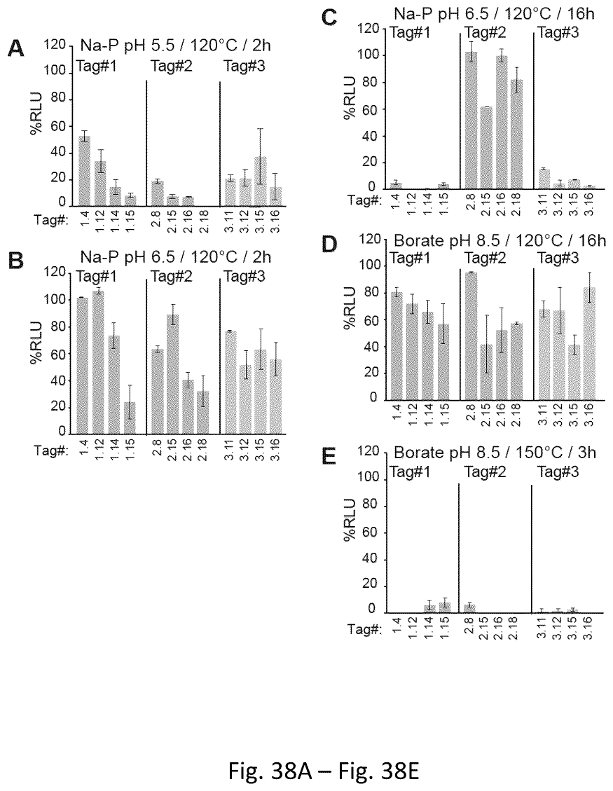

SELDA was then used to evaluate the ligation efficiency of DNA tags that underwent thermal exposure after being resuspended in different buffers, at different pHs. All DNA tags resuspended in Sodium-phosphate buffer (250 mM, pH 5.5) showed a dramatic decrease in the ligation efficiency after incubation at 120° C. for 2 hours (#1.12: 33.8%, #1.14: 14.6%, #1.15: 8.3%, #2.8: 19.1%, #2.15: 7.5%, #2.16: 7.1%, #2.18: 0%, #3.11: 21.1%, #3.12: 21.5%, #3.15: 37.6% and #3.16: 14.8% RLU) (FIG. 38A). Remarkably, using similar experimental conditions and increasing the pH value by only 1 unit (pH 6.5) the DNA ligation efficiency of the same DNA tags was almost back to normal (#1.4: 102%, 1.12: 107%, #1.14: 73.6%, #1.15: 24%, #2.8: 63.5%, #2.15: 89.3%, #2.16: 40.9%, #2.18: 32.4%, #3.11: 77%, #3.12: 51.7%, #3.15: 63.5% and #3.16: 56.2% RLU) (FIG. 38B). When repeating this latest experiment for 16 hours this time, DNA tags from Tag #1 and Tag #3 series showed low to null DNA ligation efficiency (#1.4: 5.2%, #1.12: 0%, #1.14: 0%, #1.15: 4.1%, #3.11: 15.4%, #3.12: 4.8%, #3.15: 7.3% and #3.16: 2.8% RLU). Surprisingly, the tags from Tag #2 series were still able to ligate efficiently under those conditions (#2.8: 102.8%, #2.15: 62%, #2.16: 100.1%, #2.18: 81.9%) (FIG. 38C). The DNA ligation efficiency was not dramatically affected when the DNA tags were resuspended in Borate buffer (150 mM, pH 8.5) and subjected to an incubation at 120° C. for 16 hours (#1.4: 80.6%, #1.12: 71.9%, #1.14: 65.8%, #1.15: 57%, #2.8: 95.3%, #2.15: 41.9%, #2.16: 52.2%, #2.18: 57.4%, #3.11: 67.9%, #3.12: 66.7%, #3.15: 41.6% and #3.16: 84.2% RLU (FIG. 38D). However, 3 hours of incubation at 150° C. abolished the ligation of all DNA fragments tested (FIG. 38E). All respective non-treated controls were set at 100% RLU.

### Example 8: Reversible DNA Alteration to Allow Solubility in Organic Solvents: Applications for DNA Encoded Library Technology

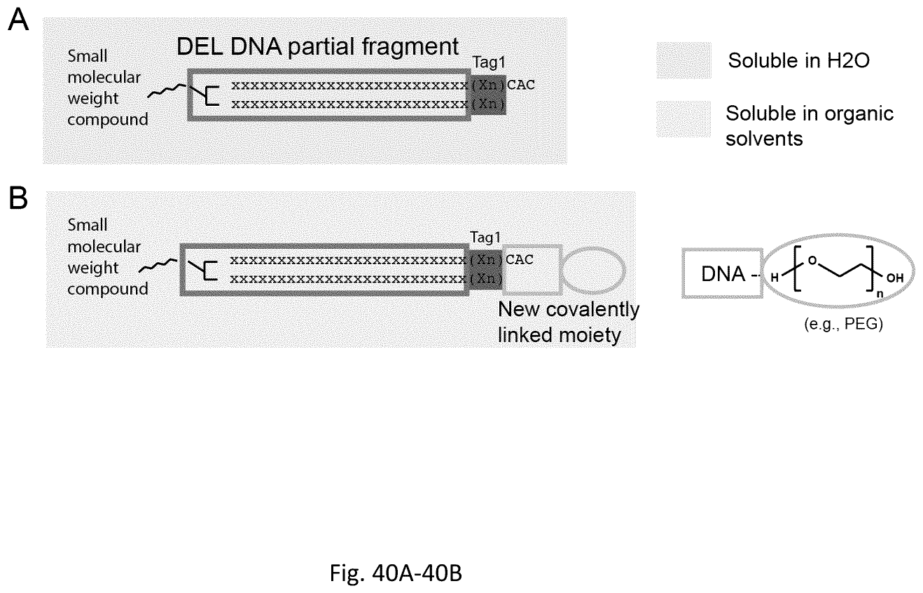

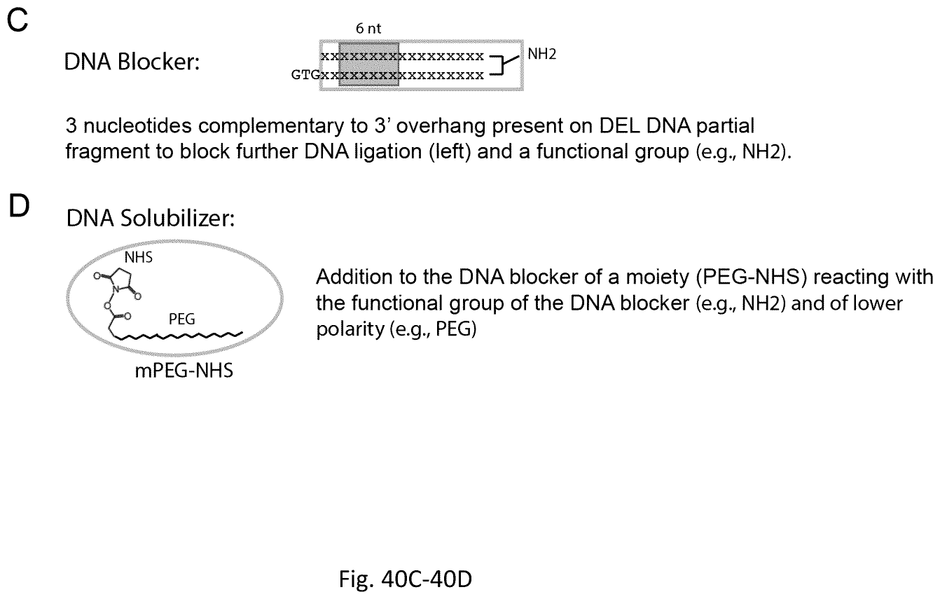

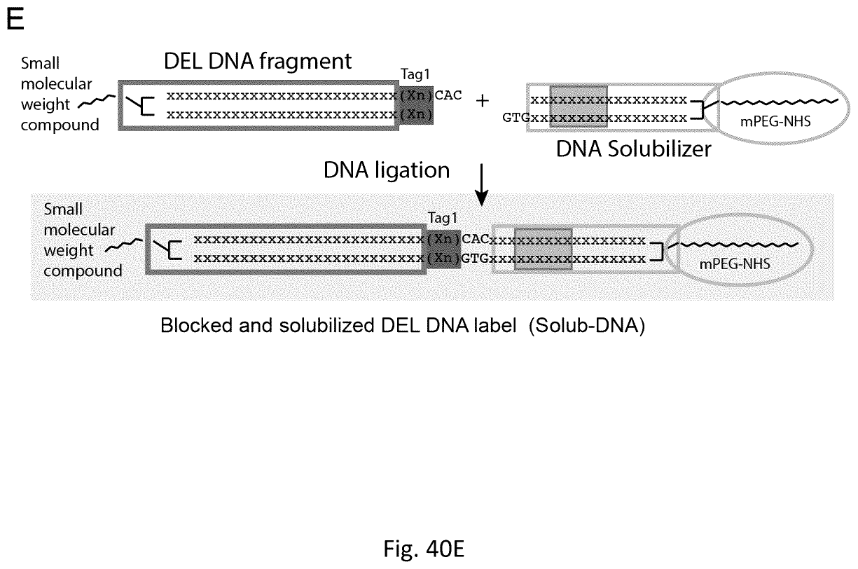

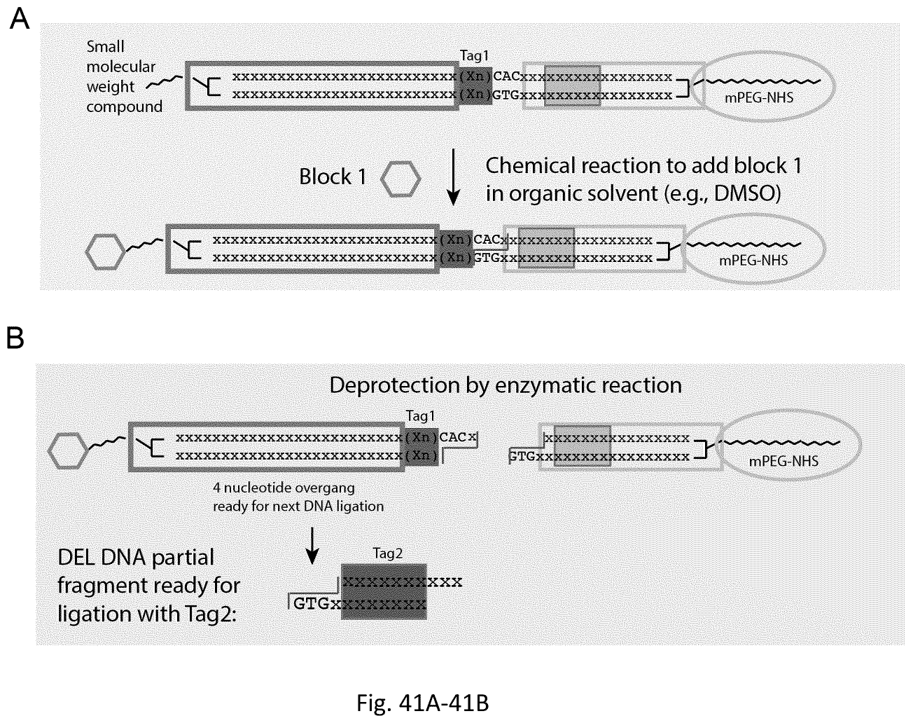

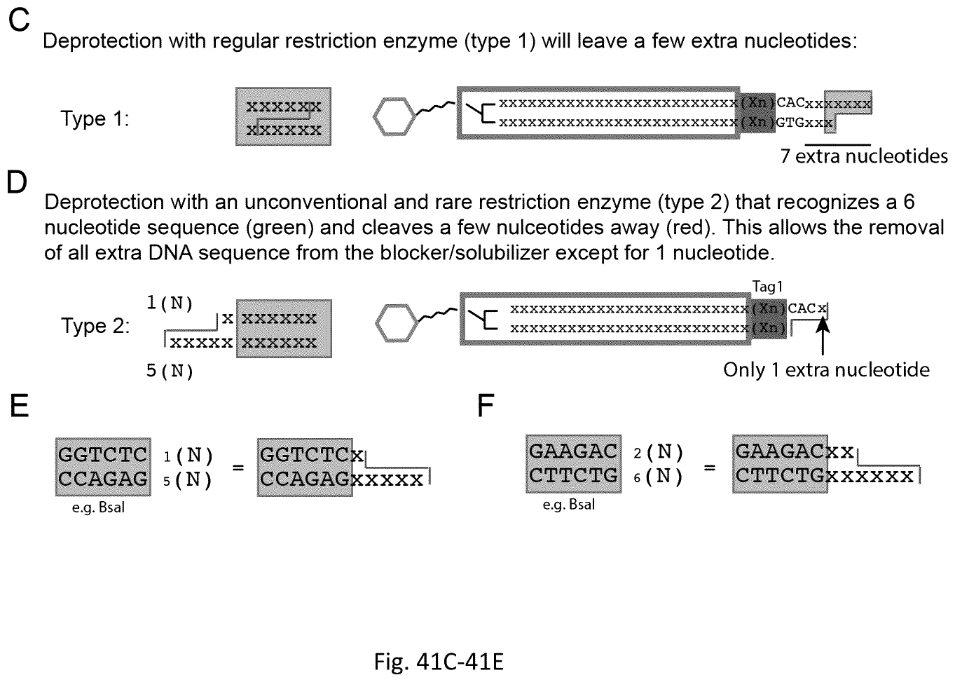

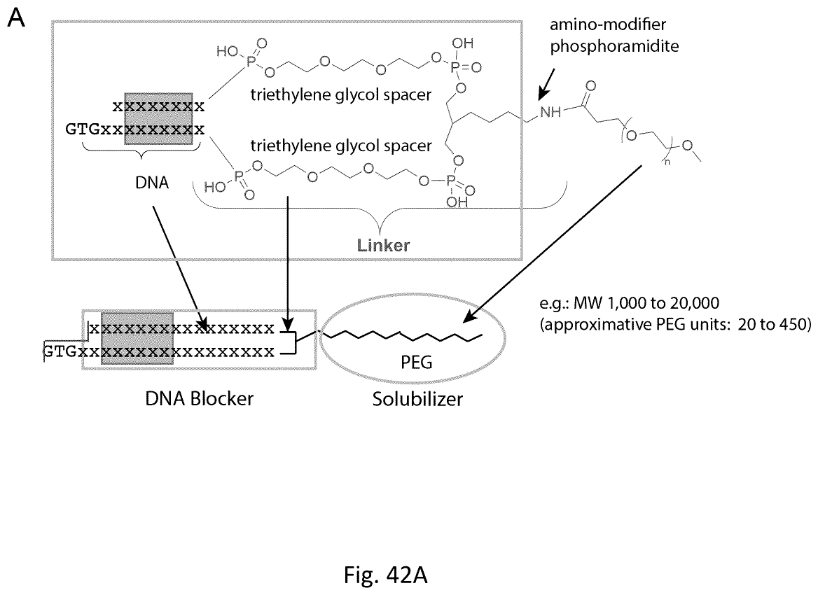

DNA and nucleic acids in general have nucleic acid phosphate groups create an overall polar backbone that results in a strong hydrophilicity. This study aimed at modifying the DNA properties in a reversible way by ligating a hybrid DNA molecule to a DNA fragment that needs to be altered. A schematic representation of the steps involved are presented in FIG. 40 and FIG. 41.

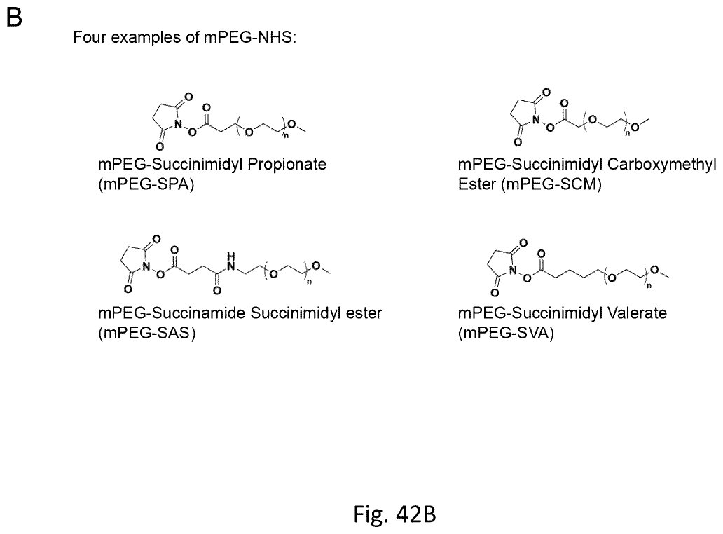

First, a short DNA fragment was used to validate the solubilization concept. Then, a DNA fragment compatible with the construction of a DEL platform corresponding to a head piece fragment linked to a Tag1 sequence, was used as proof of concept as shown in FIG. 40A. This fragment, called DEL DNA partial label, was chosen to evaluate the impact of reducing its polarity to allow DNA solubilization in organic solvent. This DNA fragment is soluble in water (blue background on FIG. 40A and later on) and it is not soluble in organic solvents including ethanol 100%. The DEL DNA partial label was then fused using T4 DNA ligation to a binary entity (FIG. 40B, yellow empty rectangle and oval shapes) that is a hybrid between DNA (rectangle) and a chemical compound (oval) to be tested for its solubilizing capacity. The light-yellow background (FIG. 40B and later on) indicates solubility in organic solvents. An example is shown on the right where the chemical compound selected is polyethylene glycol (PEG). The DNA moiety used to bridge the DEL label with the solubilizing compound altering DNA properties has been called “DNA Blocker” (FIG. 40C) as it blocks the DEL label from further DNA ligations. Once the DNA Blocker and the solubilizing compound, referred to herein as the “DNA solubilizer” (e.g., PEG)(FIG. 40D) have been covalently linked, a final step was performed in which the two DNA pieces (DEL DNA partial label+DNA solubilizer) were ligated using T4DNA ligase. The final product contains the DNA fragment to be modified, DEL DNA partial label, and the DNA blocker fragment linked to the solubilizer, and was called osDNA (FIG. 40E). This fragment now theoretically soluble in organic solvent can be used as substrate for a chemical reaction requiring an organic solvent to attach a chemical block onto the scaffold present on the DEL DNA partial label (FIG. 41A). The final step, after the chemical block has been added, is to reverse the process and release the DEL DNA partial label from the DNA blocker. An enzymatic reaction allowed a precise DNA cleavage (FIG. 41B; red lines); in this case the cleavage presented was engineered for a restriction enzyme of type 2. Following the cleavage, the resulting DEL DNA partial label is ready for ligation with another DNA fragment/acid nucleic (e.g., Tag2) (FIG. 41B, red rectangle) for the next step of the split-and-pool approach. Of note, the release DEL DNA partial label harbors 4 overhang nucleotide after cleavage, which has an added advantage as it differentiate DEL DNA partial label that have not been modified and will have still only 3 nucleotides overhang. The deprotection of DEL DNA partial label by restriction enzymes allows for most of the DNA blocker fragment to be removed. Deprotection of the DEL DNA using a regular restriction enzyme (type 1) implies that the cleavage site is overlapping with the recognition site (FIG. 41C; red line inside the green box). The scheme indicates that in that case 7 extra nucleotides will be added to the DEL label each time a DNA blocker is removed that way. At the contrary, using a less common enzyme (type 2 as previously shown) that cleaves the DNA outside the recognition sequence (FIG. 41D, E; red line outside the green box) will lead to only 1 or 2 extra nucleotide(s) being added. Two examples of type 2 enzymes are shown: BsaI with a cleavage site 1 and 5 nucleotides away from the recognition sequence (1 extra nucleotide remains after enzymatic cleavage) and BbsI cutting 2 and 6 nucleotides away from the recognition sequence (2 extra nucleotides remain after enzymatic cleavage). An example of chemical linker used to bridge the DNA blocker and the solubilizer is presented with molecular resolution (FIG. 42A) and four examples of PEG are presented in FIG. 42B.

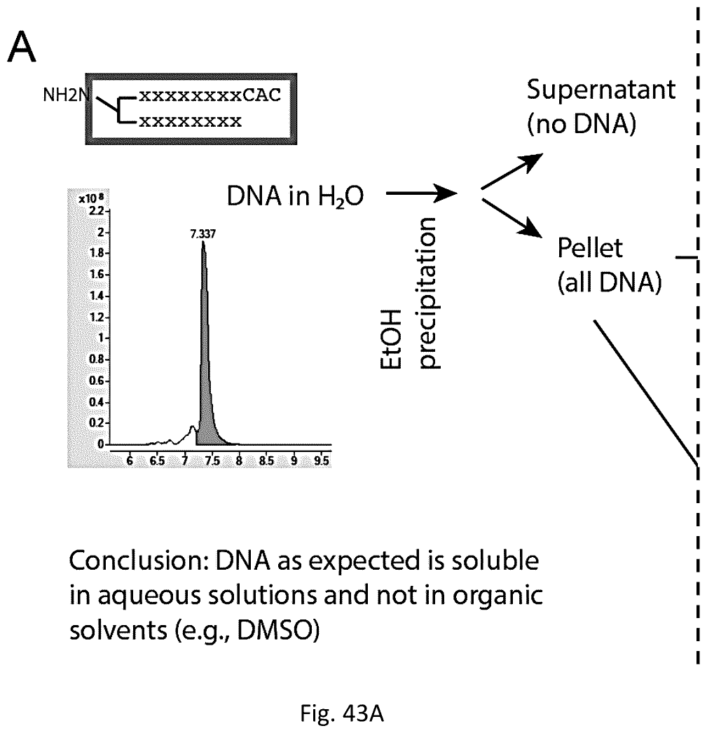

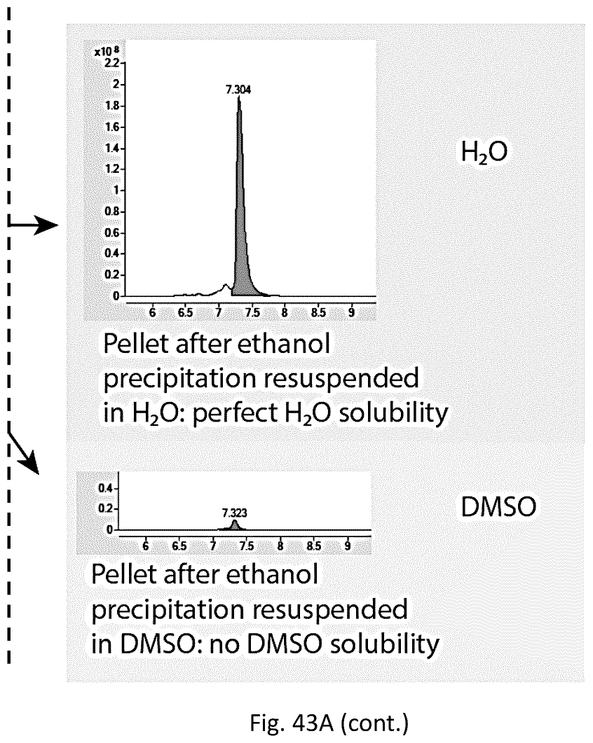

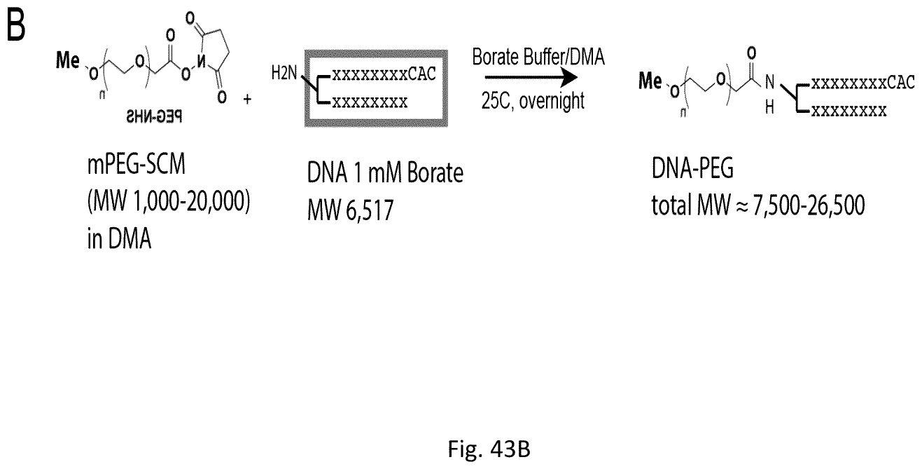

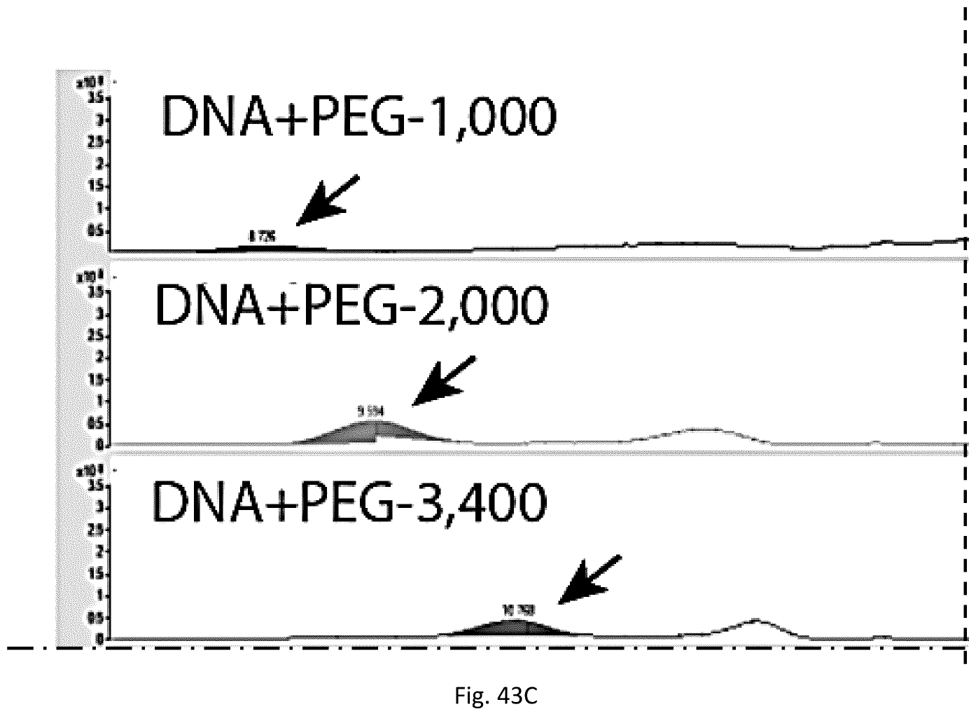

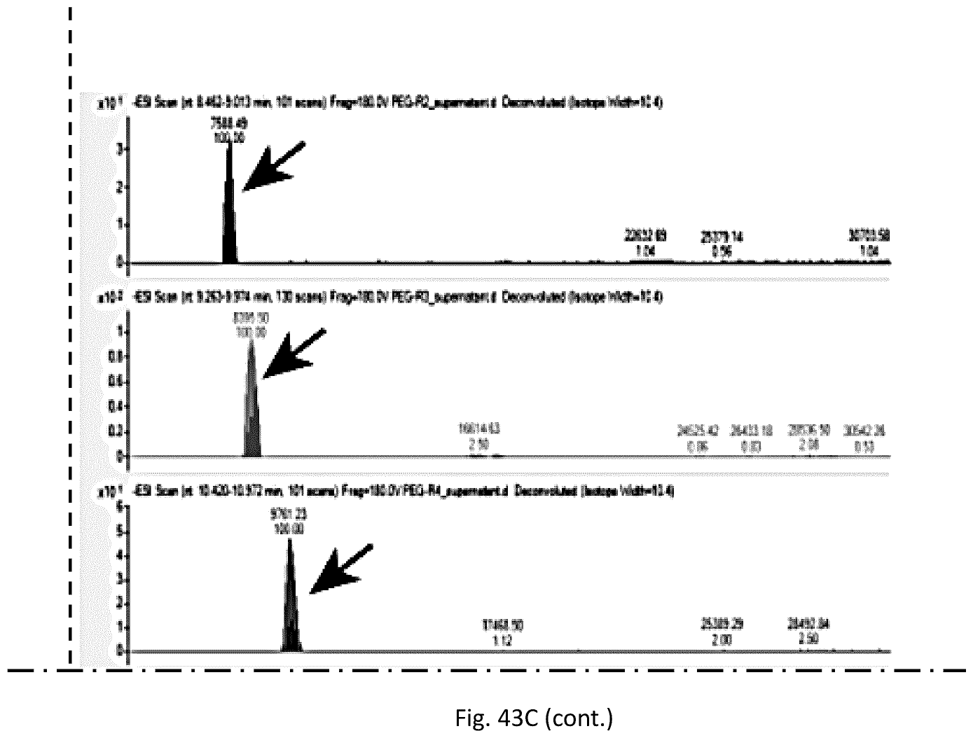

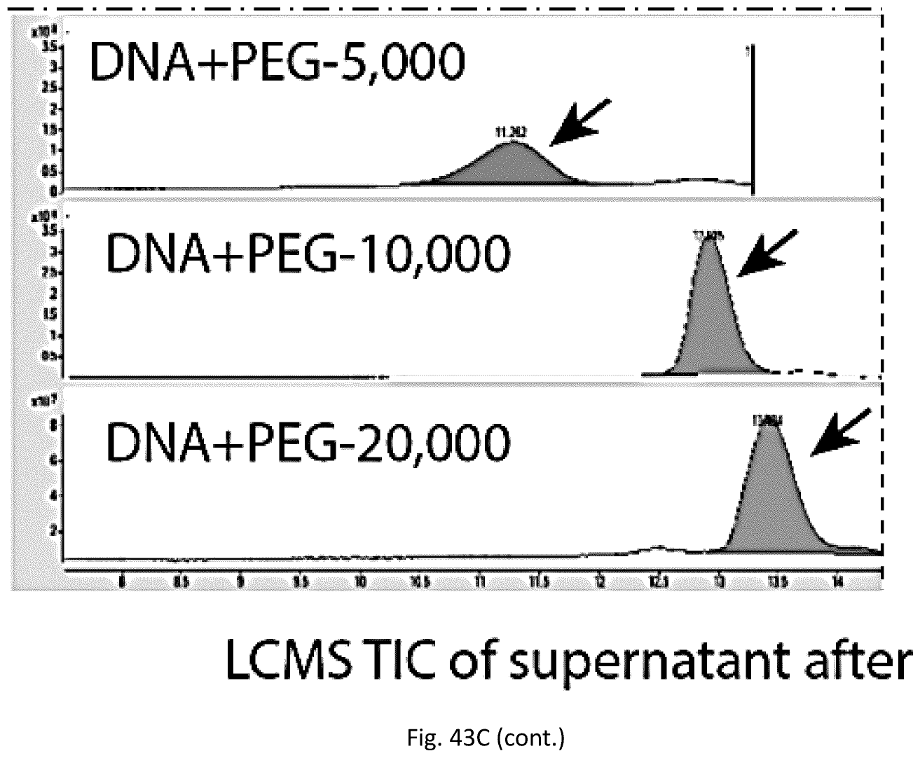

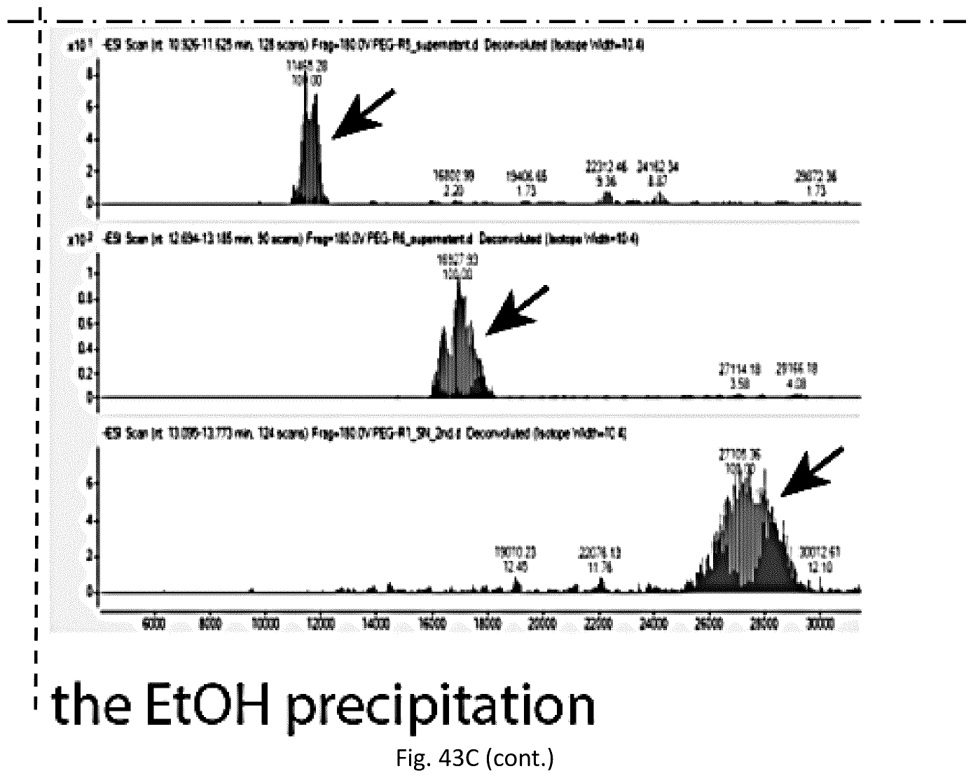

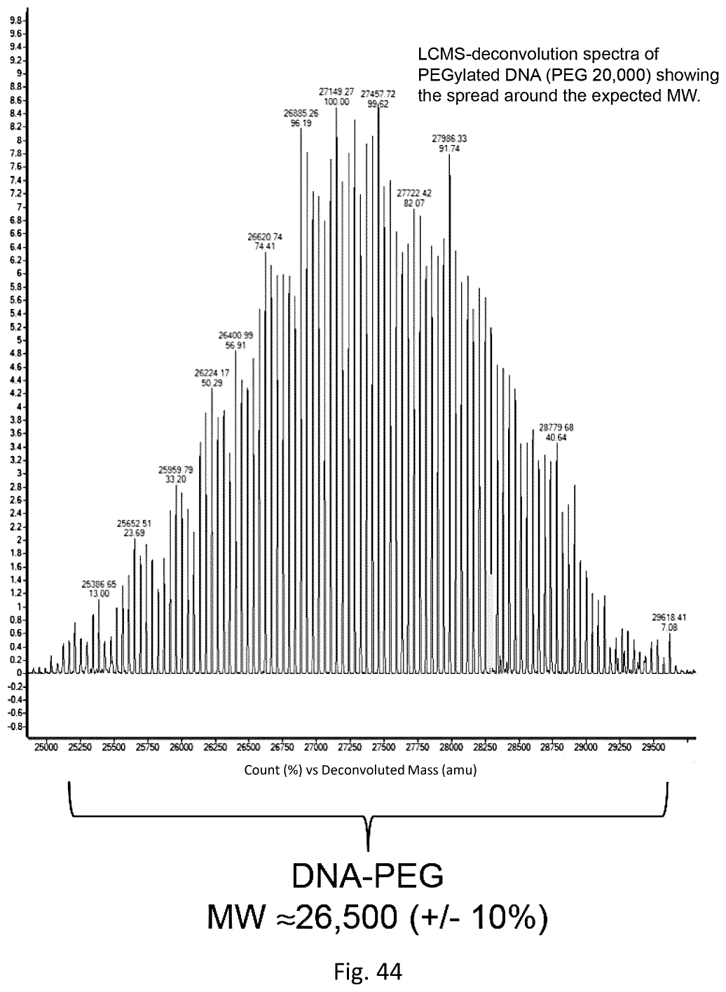

The actual solubility of a short DNA fragment (FIG. 43; Top, left) was evaluated in water and in an organic solvent. The DNA in water was ethanol precipitated and the pellet dried. As expected, the DNA resuspended well in water and not at all in DMSO. The visual results were confirmed by LCMS analysis. This established our basic protocol to evaluate DNA solubility in water (FIG. 43A; left spectrum is identical to blue background spectrum) and very poorly soluble in DMSO (FIG. 43A; yellow background). A x-nucleotide long DNA blocker was linked to six different mPEG-SCM molecules with a molecular weight (MW) ranging from 1,000 to 20,000, for a final MW of 7,500 to 26,500 respectively. First, to validate the concept, we chose the smallest piece of double strand DNA (dsDNA) that is relevant for DEL construction as shown in FIG. 43A (Top, left). The test DNA fragment in borate buffer is then linked covalently to six different mPEG-SCM molecules (mw: 1,000; 2,000; 3,400; 5,000; 10,000; 20,000) harboring approximately 20 to 450 PEG units (FIG. 43B). After addition of PEG-N-hydroxysuccinimide (PEG-NHS) ester to the DNA, the samples were ethanol precipitated overnight and the supernatant (EtOH) were separated. The pellets were dried and used for the next step. A solubility test of each six PEGylated DNA was carried out by measuring retention in the supernatant, where no DNA should be found as shown previously (FIG. 43A). LCMS analysis of supernatant revealed that DNA can actually be found in the ethanol phase (FIG. 43C, top 2 spectra), including for the smallest PEG molecule added indicating that 20 PEG (mw 1,000) units is already efficient to solubilize DNA (see arrows), at least partially. More importantly, the solubility of DNA in ethanol increased proportionally to the size of the PEG molecule fused with the DNA fragment taking advantage of a free amine (NH₂) group (FIG. 43C, from top to bottom spectra). These results clearly indicate a PEG moiety linked to DNA significantly alters its polarity and PEGylated DNA molecules are soluble in organic solvents. Due to the nature of PEG synthesis (in vitro using an enzyme) it is not possible to control exactly the size of PEG molecules. Therefore, their molecular weight is indicative of the average size, but it is a mixture of molecules of different sizes +/−10% of the announced MW (FIG. 44).

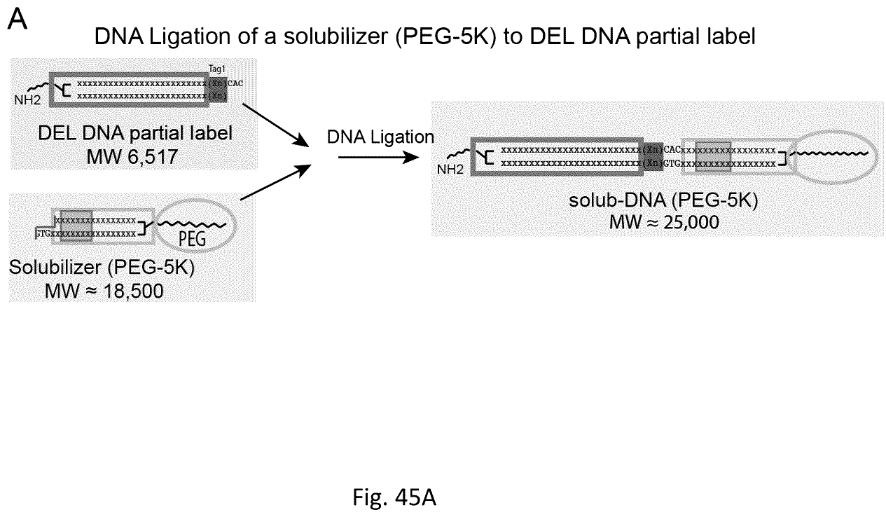

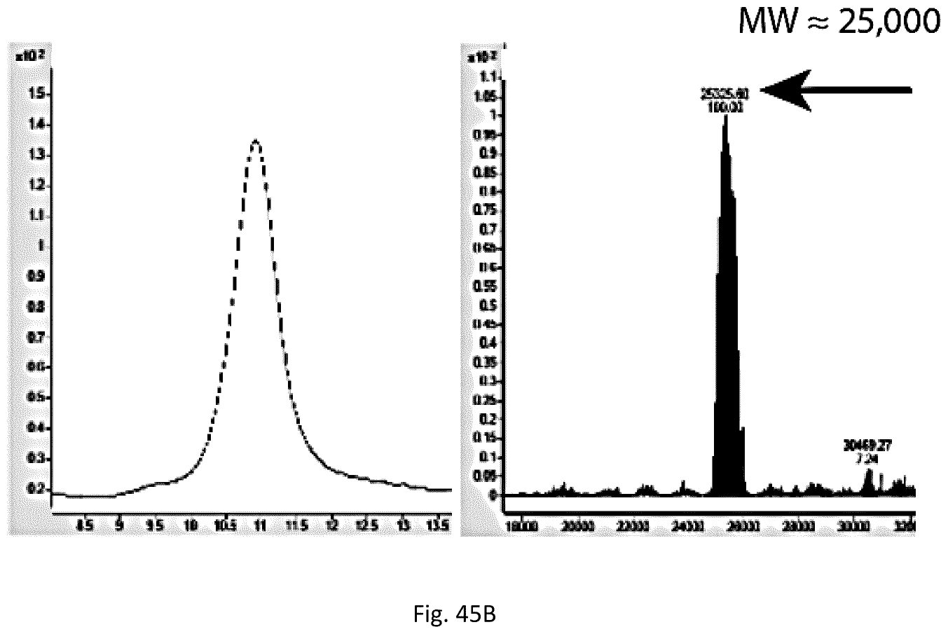

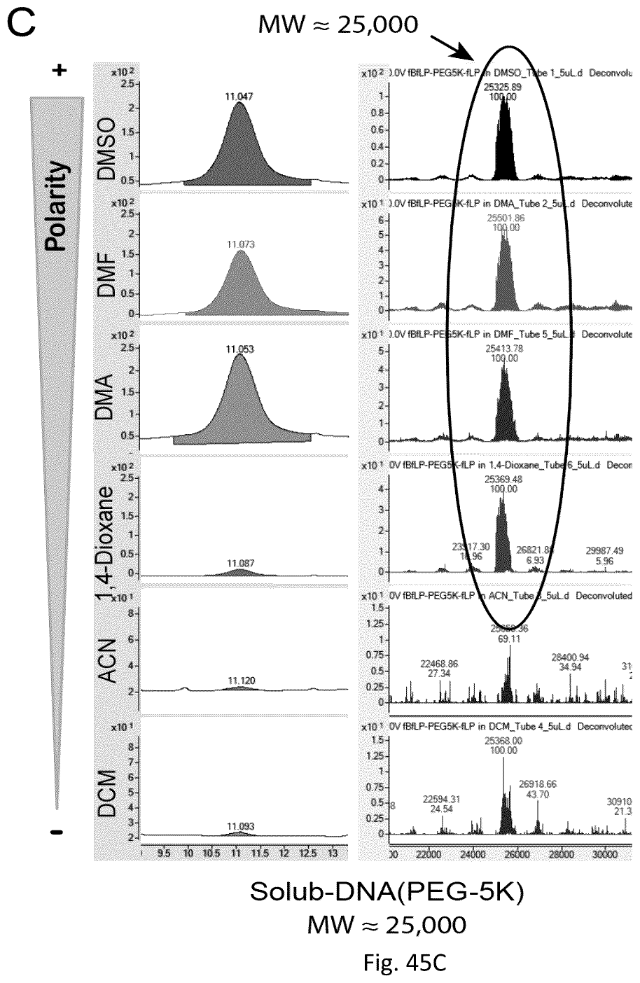

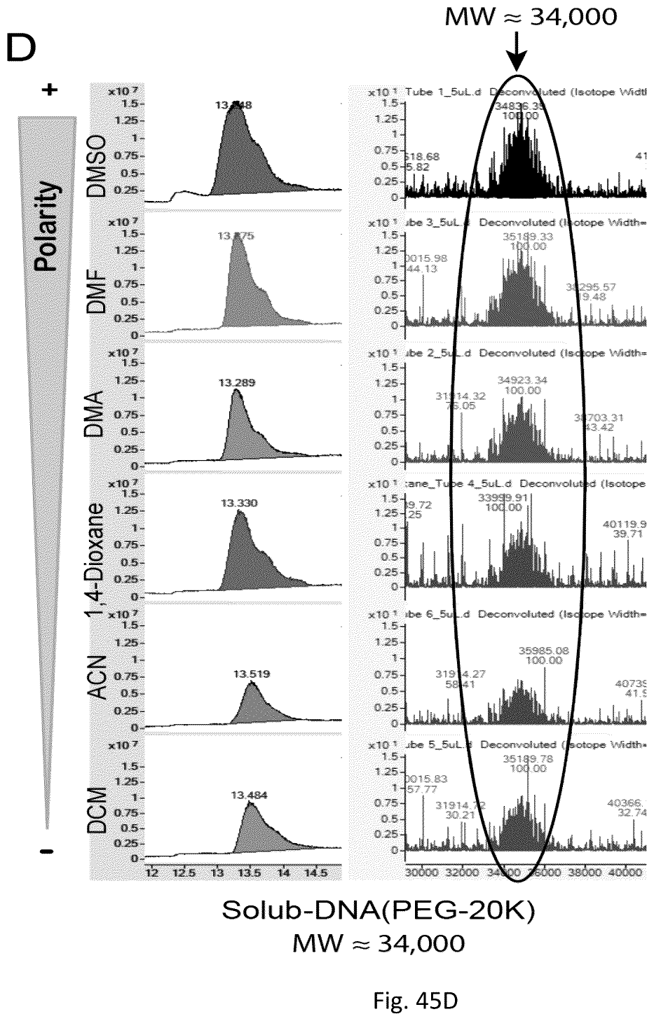

Once the solubilization proof-of-concept validated on a short DNA fragment, a series of experiments were performed in similar conditions to what is shown in FIGS. 40-41, and to match entirely a strategy DEL compatible, actually reproducing one cycle that would be used in a split-and-pool strategy to build a DEL. When supernatants were analyzed previously (FIG. 43C), a satisfying solubility level was achieved with PEG MW 5,000 or larger. PEG MW 5,000 was chosen for subsequent experiments as it will allow better detection of future modifications compare to molecules made with PEG 10,000 and PEG 20,000. The DNA ligation reaction between DEL DNA partial label (MW 6,517) and the DNA solubilizer (PEG 5,000; MW 18,500) taking advantage of a free amine (NH2) group was first tested and optimized. An organic-soluble DNA (osDNA) fragment (MW 25,000) as shown in FIG. 45A was successfully generated. The solubility of osDNA was evaluated first by LCMS after ethanol precipitation and by analyzing the ethanol phase (supernatant) confirming that osDNA was well soluble in ethanol (FIG. 45B). Once the ratio and conditions determined, the reaction was scaled-up and the resulting desired product (osDNA) was purified by HPLC, aliquoted in several tubes and lyophilized fully. These osDNA pellets were then dissolved tentatively in 6 different solvents of decreasing polarity (DMSO, DMF, DMA, 1,4-dioxane, ACN and DCM). After overnight resuspension, the solubility of these samples was analyzed using LCMS-UV spectra. Interestingly, the modified PEGylated DNA was found to be completely soluble in DMSO, DMF and DMA, partially soluble in 1,4-dioxane and not soluble in ACN and DCM (FIG. 45C). These positive results are highlighted with the open oval shape on the deconvoluted spectra. More importantly, similar experiments were repeated with now using the largest PEG moiety (MW 20,000) as described above (FIGS. 45A, B and D) and in these conditions, the osDNA pellets were completely soluble in all 6 solvents tested (FIG. 45D).

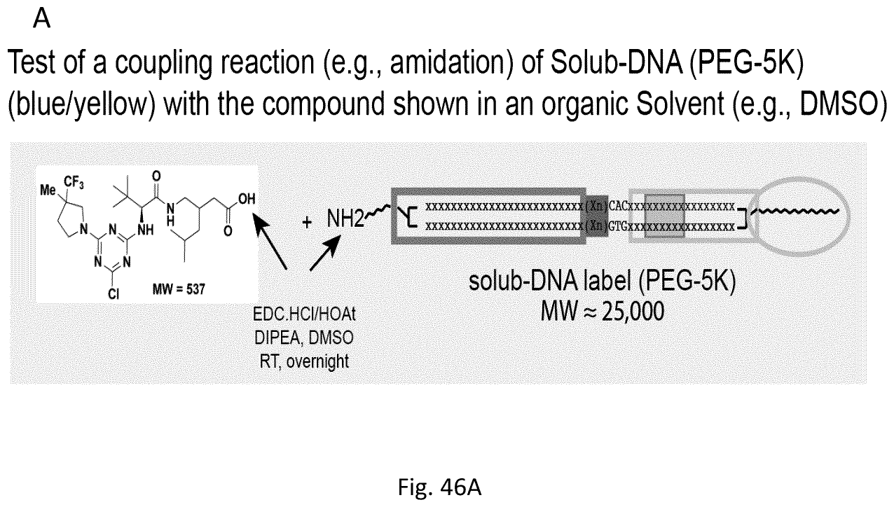

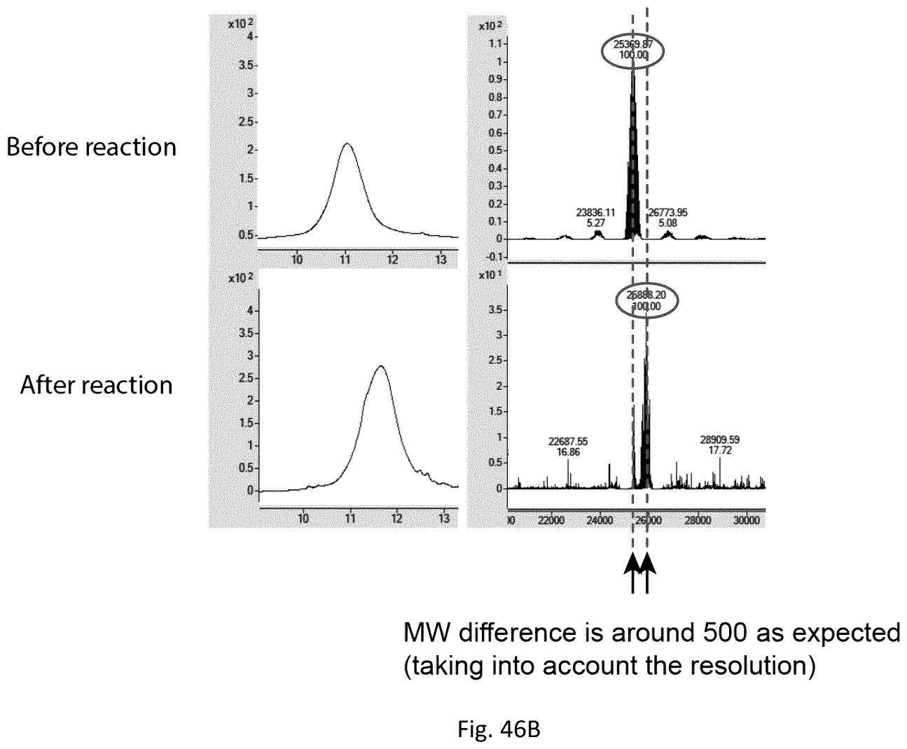

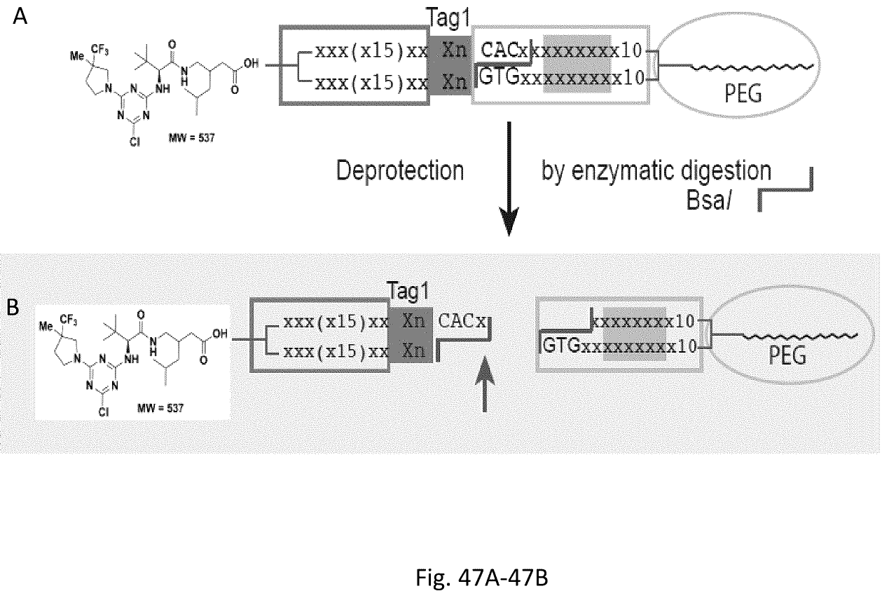

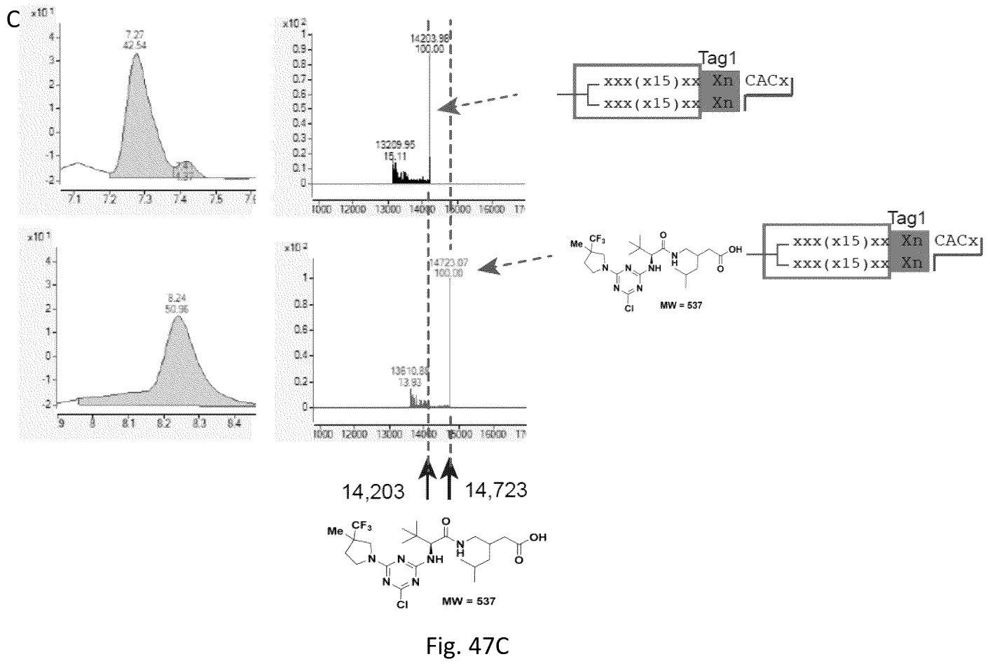

Based on these results, having demonstrated the full solubility of PEGylated DNA in organic solvent such as DMSO, one of the last two important remaining step was to perform a chemical reaction (e.g., amidation) between osDNA and a chemical acid block in DMSO exactly as it would be done to construct a DEL, to add the chemical block onto a chemical scaffold or a nascent molecule. The DNA in DMSO was mixed with 100 equivalents of the acid block (FIG. 46A; MW 537) in DMSO followed by addition of 100 equivalents of EDC·HCl/HOAt and 300 equivalents of DIPEA in DMSO. The reaction mixture was stirred using a ThermoMixer C at 900 RPM at room temperature overnight. The LCMS analysis of the crude reaction mixture showed that the reaction proceeded nicely. A new peak was observed at around MW 25,800. There is a complete shift in a peak with nearly 500 molecular weight difference (FIG. 46B) and the shift has been highlighted with the red and blue dotted lines. This has been also successfully performed and confirmed using PEG MW 5,000 Da (5K) and 20,000 (20K).

### Expansion of Amidation Reaction to Additional Compounds

Once the amidation reaction was successful with compound A1, the scope and generality of this coupling reaction was further evaluated by varying the acids (Table 2) followed by digestion with Bsa I. The reaction was facile with aliphatic acids A2 and A3, resulting in yields of 60% and 90%, respectively. The coupling reaction of aromatic acids A4-A8 with PEGylated organic-soluble DNA (osDNA) proceeded well, with very good yields. Finally, the heteroaromatic carboxylic acids A9 (Indole-5-carboxylic acid) and A10 (pyrazole carboxylic acid) were also tested successfully and produced very good yields. The benzothiophenes carboxylic acid A11 led to a 4500 yield. As a result, it is possible to use an organic solvent (DMSO) as the reaction solvent for PEGylated osDNA. This has important applications for water-sensitive organic reactions on DNA.

TABLE 2
Acids tested in the amidation reaction

| Entry | Acid | Acid structure | % Yield |
|---|---|---|---|
| 1 | A1 | 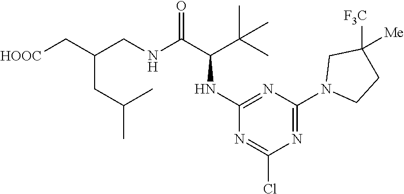 [SMILES: CC(C)CC(CNC(=O)[C@H](Nc1nc(Cl)nc(N2CCC(C)(C(F)(F)F)C2)n1)C(C)(C)C)CC(=O)O] | 48% |
| 2 | A2 | 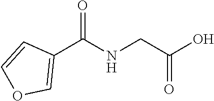 [SMILES: O=C(O)CNC(=O)c1ccoc1] | 60% |
| 3 | A3 | 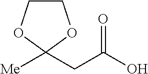 | 90% |
| 4 | A4 | 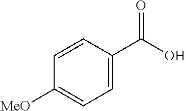 [SMILES: COc1ccc(C(=O)O)cc1] | 95% |
| 5 | A5 | 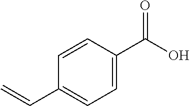 | 91% |
| 6 | A6 | 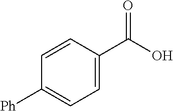 [SMILES: O=C(O)c1ccc(-c2ccccc2)cc1] | 69% |
| 7 | A7 | 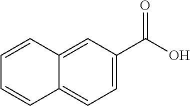 [SMILES: O=C(O)c1ccc2ccccc2c1] | 86% |
| 8 | A8 | 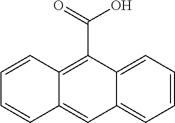 [SMILES: O=C(O)c1c2ccccc2cc2ccccc12] | 80% |
| 9 | A9 | 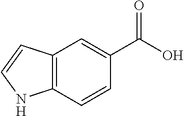 [SMILES: O=C(O)c1ccc2[nH]ccc2c1] | >95% |
| 10 | A10 | 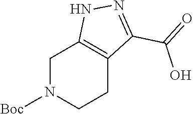 [SMILES: CC(C)(C)OC(=O)N1CCc2c(C(=O)O)n[nH]c2C1] | 79% |
| 11 | A11 | 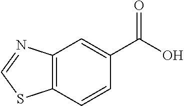 [SMILES: O=C(O)c1ccc2scnc2c1] | 45% |

Finally, the last step was the deprotection of the DEL DNA partial label by enzymatic digestion to allow for the next DEL split-and-pool step. The PEGylated DNA following chemical reaction is lyophilized, resuspended in water and processed for enzymatic digestion using standard conditions. The type 2 BsaI restriction enzyme was used as shown in FIG. 41 D-E to allow for a very precise removal of the blocker DNA. Similarly, BbsI was used as a second example and also successfully liberated the DEL DNA partial label.

The materials and methods used for the experiments are described below.

### Reagents:

The following commercially available reagents were used: Acetonitrile (HPLC grade, cat #34851, Sigma-Aldrich, USA), methanol (HPLC grade, cat #A452SK-4, Fisher Scientific, USA), NN-dimethyl formamide (DMF) (HPLC grade, cat #588725, Sigma-Aldrich, USA), NN-dimethyl acetamide (DMA) (HPLC grade, 99.5%, cat #22916, Alfa Aesar, USA), 1,4-dioxane (HPLC grade, cat #296309, Sigma-Aldrich, USA), Dimethyl sulfoxide (DMSO) (>99.5%, cat #D5879, Sigma-Aldrich, USA), 1,1,1,3,3,3-Hexafluoroisopropyl alcohol (99.9%, cat #00080, Chem-Impex international, Inc., USA), Triethylamine (>99%, cat #T0886, Sigma-Aldrich, US), N,N-Diisopropylethyl amine (>99%, cat #T0886, Sigma-Aldrich, US). UltraPure distilled water (DNAse, RNAse free, cat #10977-015, Invitrogen, USA) and Sodium hydroxide solution (BioUltra, 10 M in H₂O, cat #72068, Sigma-Aldrich, USA) were used for buffer preparation. Deionized water was used for LCMS mobile phase preparation. Activated polyethylene glycol mPEG-SCM (PEG-NHS ester, molecular weight of PEG=5,000) was purchased from Biopharma PEG Scientific Inc, USA. The other PEG-NHS esters of different molecular weight (1,000; 2,000; 3,400; 10,000; 20,000) were also purchased from Biopharma PEG Scientific Inc., USA.

### DNA Design and Preparation:

All DNA fragments used in the present work were designed in-house, custom made and synthetized by Integrated DNA Technologies, Inc. (IDT, Coralville, Iowa, USA). Lyophilized DNA samples were resuspended in Tris-EDTA (TE) buffer pH 8.0 at 1 mM (or unless specified at lower concentration and/or in H2O), tested for quality purpose by mass spectrometry (LC/MS), quantified and stored at −20° C. The sequence and modifications of the DNA presented and used in FIG. 43 continued iSp9, an internal triethylene glycol Spacer 9 and Uni-Link™ (iUniAmM), an amino-modifier phosphoramidite harboring a free primary amine attached to the 5′-end of an oligonucleotide via an aliphatic spacer arm. However, these components can be substituted for other systems that would link two single DNA fragments covalently and present a functional group (e.g., NH2)).

### LCMS Instrumentation:

LCMS analyses were performed using an Agilent LCMS system (LCMS-TOF 6230B) (Agilent, Santa Clara, CA, USA) according to the manufacturer instructions consisting of LC parts, a multisampler (model number—G7167A), binary pump (model number -G7112B), column compartment (model number -G7116A) and UV/MWD detector (model number—G7165A), and MS TOF (model number—G6230B).

### LCMS Analysis Conditions:

The mobile phase consisted of 100 mM HFIP and 8.9 mM TEA in deionized water (A) and MeOH (B). The samples were injected onto a reverse phase chromatography column (Targa C18, 5 μm, 50×2.1 mm, 120 A°), and gradient elution was as follows: 1% B hold for 1 minute; 1%-95% B for 15 minutes and set the post time for 3 minutes to equilibrate; at a flow rate of 0.4 mL/min and the column temperature at 40° C. The Dual ESI negative mode polarity was used with scan range of 500-3200 Da. The source conditions were as follows: Drying gas flow 12 L/min at 325° C. and a nebulizer pressure of 30 psi. The capillary voltage was set to 4000V.

### LCMS Data Acquisition and Analysis:

The data for each DNA sample were acquired using Agilent mass hunter workstation data acquisition software and the data were analyzed using Agilent mass hunter qualitative analysis B.07.00. The quality and estimated yield of DNA samples were determined by examination of the UV absorbance traces at 260 nm and Total Ion Chromatogram (TIC) traces corresponding to the peaks after deconvolution.

### PEG-DNA Synthesis:

To the DNA oligonucleotides (1 mM) resuspended in 150 mM borate buffer (pH 9.5) was added the PEG-NHS ester (20 mM) in DMA at room temperature and the reaction mixture was stirred (ThermoMixer C) at 900 rpm at 25° C. overnight. The resulting product was purified by reverse-phase high performance liquid chromatography (Gemini C18 column, 100×10 mm inner diameter, 5-μm particle size, Phenomenex, USA). The acetonitrile/MeOH concentration was increased from 1% to 95% over 15 min and 95% to 100% over 20 min. Unreacted DNA was eluted at around 4-5 min, and PEGylated DNA fragments were eluted at around 8-10 min for (PEG-5,000) and 10-12 min for (PEG-20,000). The solution containing PEG-DNA was collected selectively and lyophilized overnight.

### DNA Ligation:

Standard molecular techniques were used for DNA ligation. Commercially available T4 DNA ligase (NEB, Biolabs) was purchased and tested using an in-house linear plasmid. DNA ligations were performed for 1 hour at room temperature using the ligase buffer provided with the enzyme. The ligation was monitored by agarose gel electrophoresis and also by LCMS when high sensibility was needed.

### Solubility of PEGylated DNA in Organic Solvents:

PEGylated DNAs (10 nmol) were lyophilized in 1.5 ml Eppendorf tubes and dissolved in 40 μl solvent [dimethylsulfoxide, dimethylformamide, dimethylacetamide, 1,4-dioxane, acetonitrile and dichloromethane]. 5 μL of this stock solution was directly injected and analyzed by LCMS. All organic solvents (HPLC grade) were purchased from Sigma-Aldrich (USA). LCMS analyses were performed using an Agilent LCMS system (LCMS-TOF 6230B) (Agilent, Santa Clara, CA, USA)

Reaction Protocol for Coupling a Chemical Block with PEGylated DNA:

The PEGylated DNAs (10 μl, 1 mM in DMSO) in a 1.5 mL Eppendorf tube, were mixed with 100 equivalents of chemical block (acid block; 10 μl, 100 mM in DMSO), EDC·HCl (10 μl, 100 mM in DMSO) and HOAt (10 μl, 100 mM in DMSO). Finally, 300 equivalents of DIPEA (10 μl, 300 μM in DMSO) were added. The reaction mixture was stirred (ThermoMixer C) at 900 rpm at room temperature overnight and the crude reaction mixture was checked the the following day by LCMS.

### DNA Digestion of PEGylated DNA for Deprotection:

Standard molecular techniques were used for DNA deprotection by enzymatic cleavage. Commercially available enzymes (NEB, Biolabs) were purchased and tested using an in-house plasmid known to contain target restriction sites. Enzymatic digestion were performed for 1 hour at room temperature in the appropriate buffer. Deprotection following digestion was monitored by agarose gel electrophoresis and confirmed by LCMS when high sensitivity was needed.

### Example 9: Impact of Organic Chemistry Conditions on DNA Durability in the Context of DNA Encoded Library Technology

In this study, DNA duplexes were favored because it offers the possibility to confirm DNA intactness in conditions compatible with DEL technology. It is important to note that this short double strand DNA (dsDNA) was denaturated in the condition used (temperatures above Tm) and exist in these conditions as a single strand DNA (ssDNA). This also means that the approach undertaken is compatible with strategies employing ssDNA. Regarding the length of the DNA piece, a short sequence was preferred in order to optimize the best possible resolution by the mass spectrometry. However, one could easily substitute this fragment by DNA fragments of any length and configuration (e.g., blunt, overhang with shorter or longer sequences).

The pH range used in these studies (3.6-11.0) was based on the compatibility of DNA with various buffers. For each of the buffer tested, 2 or 3 pH were chosen in the range that is compatible with their intrinsic properties. Furthermore, different buffers were evaluated and showed some significant differences in DNA durability among buffers at the exact same pH.

In the DEL field, chemical reactions are typically performed at 100° C. or less. Here this temperature was challenged and higher ones were tested, further exploring and extending the DEL-compatible chemical space. In a previous study up to 210° C. has been tested but DNA was quickly degraded after 5 minutes starting at 130° C. Here it is shown that in some conditions 150° C. was still more than acceptable. More importantly, this study demonstrates that in many conditions intact DNA is still present as seen by LCMS even though classical agarose gel electrophoresis showed otherwise. This opens up new avenues and would allow to work in much more challenging chemical conditions, as long as intact DNA could be purified and rescued.

The percentage of GC content in the fragments provided the durability map with respect to temperatures vs. acidic conditions. At pH 6.5, higher level of degradation was observed with increasing temperatures and GC content. At the contrary, at basic pH the GC content did not affect DNA durability. For DNA fragments containing 50% GC the DNA fragments harboring a 5′ guanosine are less resistant than the fragment having only internal guanosines. The absence of purine nucleotide at the end of the sequence (either at 5′ end or 3′ overhang end) also increase DNA durability. This could lead to a situation where the DNA post-reaction is mostly intact as no purine nucleotide can be lost. In summary, it is best not to have purine nucleotides as the last nucleotide. For a given GC content, no difference was observed with different overhang nucleotides as long as the fragments contained the same purine or pyrimidine overhang nucleotides. It might sound better to work only with AT bases as observed in the sequence with 0% GC content but it is not feasible for coding purposes because of complexity and diversity needs for DEL.

Unfortunately, the non-DNA variant purine-based nucleoside inosine showed a massive DNA degradation. Higher level of dephosphorylation was observed at basic pH for DNA fragments containing inosine compared to natural nucleotides, extremely high-level of DNA degradation was observed in acidic pH. Overall, the modified DNAs tolerated well basic conditions at 100° C. for 24 hours with very minimal degradation. Degradation gradually increased with time after 24 hours and up to 50% or more of DNA was observed in sodium borate buffer after 100 hours. Remarkably, at pH 11.0 and 140° C., the DNA was highly resistant until 3 hours without any degradation.

Transition-metal-catalyzed reactions are powerful strategies for generating a variety of organic molecules including complex small molecule drugs and drug-like molecules. Few studies so far published in DEL field investigated a metal catalysts and reagents. It is known that most metal salts and organic reagents were well tolerated in terms of DNA durability in organic solvents at 40° C. when 100 equivalents were used. The high equivalence of the metal salts might lead to severe DNA degradation at high temperatures. Therefore, the equivalence had to be reduced. In organic solvent under dry conditions <1 equivalent is sufficient. Here to compensate for the reduced activity of metal catalysts in aqueous solutions, 10 equivalents were preferred.

DNA being insoluble in organic solvents, and numerous chemical reactions being incompatible with aqueous solutions, the mixture buffers/organic solvents represent a good compromise for DEL chemistry as it satisfies both the DNA solubility (aqueous buffer) and chemical reaction (organic solvent). In some cases, the ratio is important for the reaction to reach higher yields. Here 3 different ratios were chosen to take that into account. Previous work indicated that at least 20% of aqueous solvent is needed.

Group 1A & 2A metals mainly interact with anionic phosphate backbone, neutralizes the net negative charge and decrease the repulsions between DNA strands. This stabilizes the DNA and increases its melting point. On the contrary, transition metal ions bind to both backbone phosphate and nucleoside bases. These metals can form adducts or cause DNA degradation depending on the nature of metal ions, redox activity and the reaction conditions used. DNA well tolerated the metals Ni, Co, Au, Ag and Cu in all the conditions tested. No adducts and the least degradation was observed for Ni and Co. This might be due to their lower affinity towards nucleoside bases comparatively to other transition metals. The metals Ir, Ru, Rh formed higher adducts and caused DNA degradation in all the conditions used but the least adducts were found in phosphate buffer. This is indicating that the buffer/solvent system is affecting the formation of adducts. Among the Rhodium ions Rh(I) and Rh(III), the ion Rh(III) formed higher adducts and caused more DNA degradation possibly because of its higher oxidation state. The palladium catalyst sSPhos Pd G2 formed abundant adducts very quickly compared to all the catalysts used in all tested conditions possibly because of its ligand which might synergize with Pd to form adducts very quickly.

As mentioned previously, due to size and complexity of DNA molecules, it is impossible to perform LCMS analysis in presence of adducts. Independently of the size of DNA, scavengers are efficient most of the times to resolve adducts as long as the right metal/scavenger is chosen. Various metal scavengers can be used depending on the nature of metal used. For example, Sulphur-based scavengers (e.g., Sodium diethyldithiocarbamate (NaDEDTC) and 2-Mercaptoethanol (BME)) can be used for soft metals removal. Oxygen-based scavengers (e.g., Ethylenediaminetetraacetic acid (EDTA) or Triaminetetraacetic acid (TAAcOH)) are effective for metals in low or zero oxidation states while their corresponding salts are more appropriate for higher oxidation state metals. The scavenger treatment of metals Ru, Ir, Rh and Pd which formed a higher DNA adducts especially in H₂O did not remove except in Pd. Even though Pd was removed fully but higher DNA degradation was observed compared to other conditions tested. No improvement for the metals Ru, Ir and Rh post scavenger treatment that possibly a strong formation or much DNA decomposition in H₂O. At same temperature Rh (III) caused DNA decomposition in H₂O but was well tolerated to DNA in Phosphate buffer at lower pH. It is indicating that the buffer, organic solvent and their ratio could be controlled the DNA durability at given temperature. 10 metal catalysts were screened mostly all metals are soft in nature. 6 metal catalysts formed no adducts in most of the conditions tested. Metals Ir, Pd and Rh formed mostly adducts in all the conditions tested and these metals and Ru formed more adducts in water. Interestingly, some of these adducts can be recovered fully or partially but not equally for all metal catalysts and not equally for given one in four conditions used. Remarkably, water is always the worst condition and Pd adducts can be easily resolved. The oxidation state has a strong impact as demonstrated with Rh(III) vs Rh(I). It is possible based on the results presented that the complexity of the metal used and its ligands explaining the difference in the adducts formation. To resolve this, several conditions must be investigated based on the nature of the metal.

The analysis of some of these results might be further complicated due to the possibility that metals can be precipitated along DNA. One alternative would be to use functionalized silica gels as scavengers and have the advantage that they can be easily separated from the DNA by decanting or filtering. Imidazole functionalized silica can be used for removal of Ni, Co, Cu, Ir, Zn and Fe metals. DMT-functionalized silica can be used for removal of Ru and hindered Pd complexes like Pd(dppf)Cl₂.

The materials and methods used in the experiments are now described

### Reagents

Reagents commercially available: Acetonitrile (HPLC grade, cat #34851, Sigma-Aldrich, USA), methanol (HPLC grade, cat #A452SK-4, Fisher Scientific, USA), N,N-dimethyl acetamide (DMA) (HPLC grade, 99.5%, cat #22916, Alfa Aesar, USA), Dimethyl sulfoxide (DMSO) (>99.5%, cat #D5879, Sigma-Aldrich, USA), 1,1,1,3,3,3-Hexafluoroisopropyl alcohol (99.9%, cat #00080, Chem-Impex international, Inc., USA), Triethylamine (>99%, cat #T0886, Sigma-Aldrich, US). UltraPure distilled water (DNAse, RNAse free, cat #10977-015, Invitrogen, USA) and Sodium hydroxide solution (BioUltra, 10 M in H₂O, cat #72068, Sigma-Aldrich, USA) were used for buffer preparation. Deionized water was used for LCMS mobile phase preparation. All the reagents were purchased at the highest commercial grade possible and used without any further purification, unless otherwise noted.

### Preparation and Design of DNA Fragments.

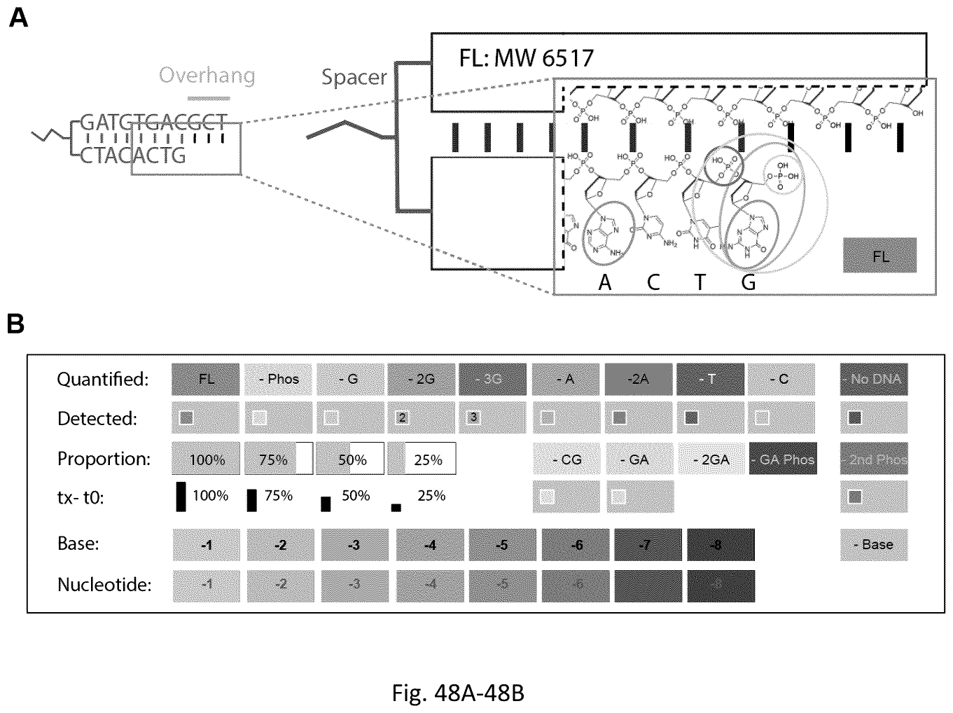

The DNA fragments (non-modified and modified) used in the present study were designed in-house, custom made and synthetized by Integrated DNA Technologies, Inc. (IDT, Coralville, Iowa, USA). The DNA fragments used were designed as shown in the results section/FIG. 48A. A phosphate residue was systematically added at the 5′ position. Lyophilized DNA samples were resuspended in Tris-EDTA (TE) buffer pH 8.0 at 1 mM, tested for quality purpose by mass spectrometry (LCMS) and stored at −20° C.

Examples of DNA modified sequences used following the design presented in FIG. 48A:

DNA 8-mer:
M1:
GTCAGACT/iSp9//iUniAmM//iSp9/AGTCTGACGC
M2:
GTCAGACT/iSp9//iUniAmM//iSp9/AGTCTGACGCT
M3:
GTTAGAAT/iSp9//iUniAmM//iSp9/ATTCTAACGCT
M4:
GGCAGCCT/iSp9//iUniAmM//iSp9/AGGCTGCCGCT
M5:
ATTATAAT/iSp9//iUniAmM//iSp9/ATTATAATGCT
M6:
GGCCGCCG/iSp9//iUniAmM//iSp9/CGGCGGCCGCT
M7:
TGCAGACT/iSp9//iUniAmM//iSp9/AGTCTGCAGCT
M8:
ideoxyI//ideoxyI//ideoxyI//ideoxyI//ideoxyI//
ideoxyI//ideoxyI//ideoxyI//iSp9//iUniAmM//
iSp9/T CTC TTC CGC T

### LCMS Instrumentation, Acquisition Conditions and Data Analysis:

Agilent instruments: LCMS analyses were performed using an Agilent LCMS system (LCMS-TOF 6230B)(Agilent, Santa Clara, CA, USA) according to the manufacturer instructions. LC components include a multisampler (model number -G7167A), binary pump (model number -G7112B), column compartment (model number -G7116A) and UV/MWD detector (model number—G7165A), and MS TOF (model number—G6230B).

Analysis Conditions: The mobile phase consisted of 100 mM HFIP/8.9 mM TEA in deionized water (A) and MeOH (B). The samples were injected onto a RP chromatography column (Targa C18, 5 μm, 50×2.1 mm, 120 A°), and gradient elution was as follows: 1% B hold for 1 minute; 1%-70% B for 12 minutes and set the post time for 3 minutes to equilibrate; at a flow rate of 0.4 mL/min and the column temperature at 40° C. The Dual ESI negative mode polarity was used with scan range of 500-3200 Da. The source conditions were as follows: Drying gas flow 12 L/min at 325° C. and a nebulizer pressure of 30 psi. The capillary voltage was set to 4000V.

Data acquisition and analysis: The data for each DNA sample were acquired using Agilent MassHunter Workstation Data Acquisition software and the data were analyzed using Agilent MassHunter Qualitative Analysis B.07.00. The quality and estimated yield of DNA samples were determined by examination of the UV absorbance traces at 260 nm and Total Ion Chromatogram (TIC) traces corresponding to the peaks.

### Protocol for Standard DNA Treatment in Different Buffers:

5 μL of DNA (1 mM H₂O) was added to 245 μl of various buffers at different pH (3.6-11.0) (as indicated in text and Figures) in 2 ml Agilent glass amber vial for a final concentration of DNA of 20 μM. The reaction mixture was then incubated at different temperatures (100° C. to 150° C., as indicated in text and Figures) for the appropriate time (15 minutes to 125 hours, as indicated in text and Figures) on a thermomixer. After incubation the tubes were kept at room temperature for 30 minutes. An aliquote was directly injected into LCMS. Before and after, as needed, DNA mixtures were stored at −20° C.

### Protocol for Other DNA Treatment in Different Buffers:

2.5 μL (1 mM H₂O) of DNA added to in 122.5 μl of various buffers at different pH in 2 ml Agilent glass amber vial for a final concentration of DNA of 20 μM. The reaction mixture was then incubated at different temperatures (as indicated in text and Figures) on a thermomixer. After incubation the tubes were kept at room temperature for 30 minutes. A DNA aliquot was directly injected into LCMS. Before and after, as needed, DNA mixtures were stored at −20° C.

### Protocol to Measure the Impact of Catalysts on DNA:

Three conditions with a buffer/DMSO ratio of 3:1 were used to test 10 catalysts: 1) 250 mM pH 6.5 sodium phosphate/DMSO (3:1); 2) 150 mM pH 9.5 sodium borate/DMSO (3:1) and 3) H₂O/DMSO (3:1). One condition with a buffer/DMSO ratio of 1:3 was also used: 150 mM pH 9.5 DMSO/sodium borate (3:1). 2.5 μl DNA (1 mM H₂O) and 2.5 μl catalyst (10 mM DMSO, 10 equivalents) were added to 120 μl of the buffer/DMSO solution at the appropriate ratio for a final concentration of DNA of 20 μM. The reaction mixture was incubated at 100° C. on a thermomixer. After incubation the tubes were kept at room temperature for 30 minutes. 10 μl was directly injected into LCMS without DNA precipitation. For scavenger treatments, 5 μl Sodium diethyldithiocarbamate (100 mM H₂O) or 2-Mercaptoethanol (100 mM H₂O) were added to 10 μl (20 μM) of the reaction mixture and then incubated at 80° C. for 30 minutes. 10 μl was directly injected into LCMS without DNA precipitation.

### Protocol to Measure the Impact of Metal Ligands and Reagents on DNA:

Two conditions with a Buffer/Organic solvent ratio of 4:1 were used to test 6 ligands and 6 reagents: 1) 250 mM pH 6.5 sodium phosphate/organic solvent (4:1); 2) 150 mM pH 9.5 sodium borate/organic solvent (4:1). The organic solvent was chosen based on ligand or reagent solubility in the buffer/solvent mixture. For both conditions, 2.0 μl fLP (1 mM H₂O) was added to 80 μl of the appropriate buffer. 20 μl of ligand or reagent (10 mM, 100 equivalents) was added to the mixture and incubated at 100° C. on a thermomixer. After incubation the tubes were kept at room temperature for 30 minutes. 10 μl was directly injected into LCMS. Before and after, as needed, DNA mixtures were stored at −20° C.

### Analysis of DNA by Gel Electrophoresis:

DNA samples were analyzed by gel electrophoresis using acrylamide and agarose gels. Two micrograms were loaded onto gels and ran 60 minutes at 90 volts. Gels were stained 15 min with Ethidium Bromide (1%) and distained 15 minutes in H₂O. Pictures were taken using an Azure instrument (Biosystems). Signals were quantified using ImageJ (NIH).

The experimental results are now described

### Presentation of the Molecular System Used to Assess DNA Durability.

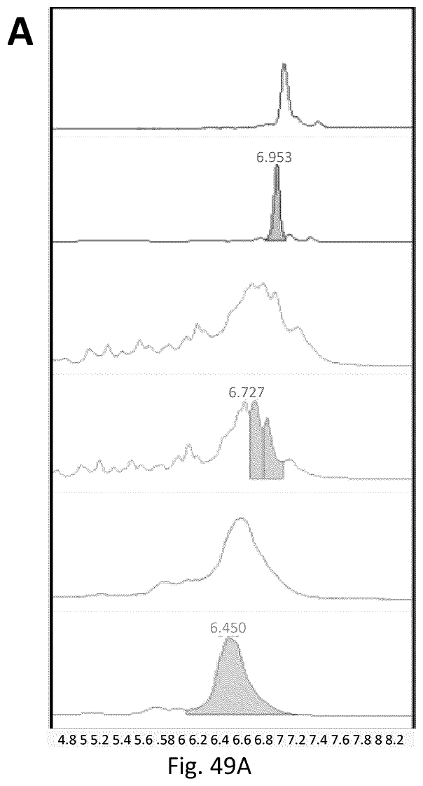

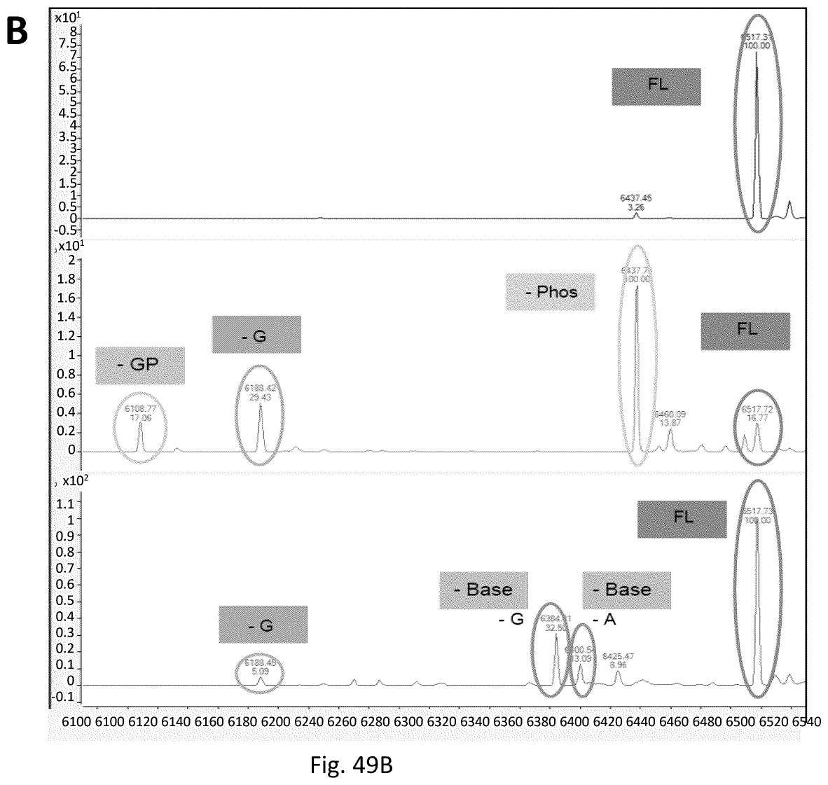

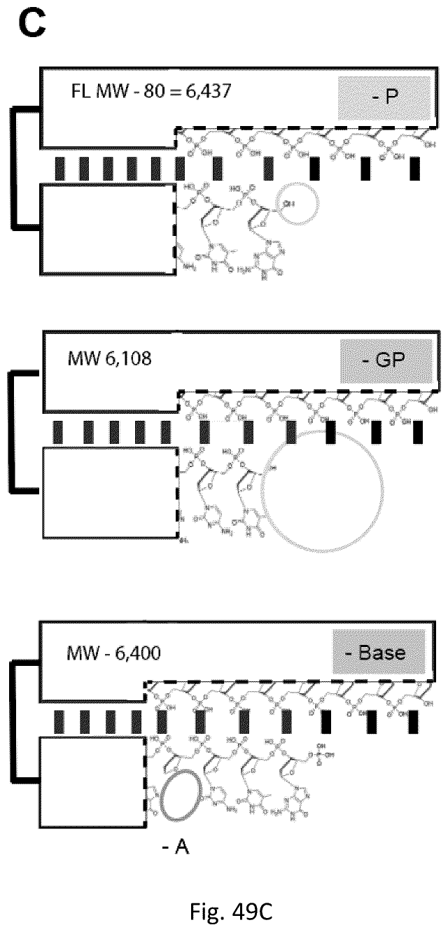

The shortest piece of double strand DNA, eight-nucleotide with three nucleotides 3′ overhang, was used for most of the studies presented unless specified. A schematic representation is shown in FIG. 48A (left). This headpiece structure is similar to what is used as starting piece to build a new DEL label. The two DNA strands are covalently linked with a spacer that carries a free functional group (e.g., NH2) for chemical addition. The headpiece allows for cohesive DNA ligation with a subsequent DNA fragment (3′ overhang and 5′ phosphate), it allows for ethanol precipitation and it is short enough (MW 6517) to be compatible with a clear LC-MS resolution following deconvolution. LCMS resolution is needed to follow every possible parameter that could be altered due to chemical conditions. These parameters are highlighted in FIG. 48A (right) and the corresponding color code matches the one used in other Figures. A color-coded legend (FIG. 48B) provides a list of all alterations measured or detected. The size of the colored rectangles is proportional to the quantity measured. For example, a full rectangle indicates that 100% of that color species was measured; a half rectangle indicates that 50% of the products measured correspond to the species associated with that color). The black vertical bars indicate the total amount of DNA found after treatment. A full vertical bar (as high as the colored rectangles) indicates that 100% of the DNA is still present. A vertical bar indicates that only 25% of total DNA was recovered, and these 25% are divided into various species found for the associated rectangle. Treatment conditions that altered those parameters and that will be further developed later were identified. The UV spectra of some of these examples are presented (FIG. 49A) and the spectra corresponding to post-deconvolution of total ion chromatograms are also provided as examples of possible DNA alterations (FIG. 49B). The unmodified DNA piece (FL) is easily identified (FIG. 49B, top), the loss of the 5′ phosphate is visible in the middle panel of FIG. 49B. More alterations are visible in the lower panel of FIG. 49B and especially the loss of an internal base (−Base, in this case a base from Adenine and Guanine). After careful analysis of all peaks present, it is possible to identify all altered species and the severity of alterations. For example, an entire nucleotide can be missing together with the second phosphate group (−GP) (FIG. 49B; middle panel). Three schematic representations of some alterations using the headpiece structure are shown (FIG. 49C).

The most used and the simplest method to evaluate DNA degradation is agarose gel electrophoresis. Four example conditions are presented and indicate that 10 hours of treatment can lead to no apparent DNA degradation (FIG. 50A, top left panel) or at the contrary to significant degradation (FIG. 50A, top right panel). In other conditions the DNA can disappear entirely at 24 hours (FIG. 50A, lower panels) and be already significantly affected as early as 5 hours (FIG. 50A, lower middle panel). However, DNA gel electrophoresis is not precise and does not allow to visualize some important parameters needed for DNA ligation such as the presence of phosphate groups and overhang nucleotides. For that reason, a DNA system will be used that allows for:
DNA annealing, as double strand is required for DNA ligation
Precipitation by ethanol or purification by other means
The possibility to ligate another DNA fragment on the free DNA end (cohesive ends are preferred as it is directional and more precise, which is required for DEL).

To illustrate this point, LCMS analysis of DNA durability in three different conditions is presented (FIG. 50B): In standard condition (Sodium Phosphate buffer, pH 8.0, 100° C.; top row), at low pH (Sodium Phosphate buffer, pH 5.5, 100° C.; middle row), and at higher temperature (Sodium Phosphate buffer, pH 6.5, 120° C.; bottom row). In standard conditions, DNA resisted well at 100° C. for as long as 24 hours with minimal impact (rectangles mostly green and black bar close to 100%). At the contrary, at pH 5.5 DNA was fairly stable up to 16 hours to finally almost disappear after 24 hours (red rectangle, middle row). At 120° C. (bottom row), after only 2 hours more than half of DNA is already dephosphorylated. At 5 hours, more than 50% of the DNA was degraded and totally dephosphorylated. At 16 hours, there is little DNA left, and new species (e.g. -G; -P-G-P) are visible. At 24 hours, all DNA is degraded (red rectangle).

After confirming the usefulness of this approach, the reproducibility of LCMS-based read out has been evaluated (FIG. 50C). The results presented correspond to three independent repeats of the same treatment performed over a period of 24 hours. The visual color coded representation clearly demonstrate that the DNA species detected are similar between the three repeats and that they are appearing in a similar time frame.

In summary, when DNA quality is not impacted, both total ion chromatogram (TIC) and UV spectra look perfectly clean with single, sharp and well delimitated peaks. At the contrary, when DNA starts degrading peaks look broad and more complex. Altogether, this approach combining gel electrophoresis and LCMS analysis allows to test and explore any parameter, and more importantly evaluate precisely if these parameters are compatible with the presence of DNA, and in what limits they could be used for chemical modifications in the DEL context.

### DNA Durability at 100° C., 120° C. and 150° C., at Different pH (3.6-11.0) in Four Different Buffers (Sodium Acetate, Sodium Phosphate, MOPS, Sodium Borate Borate).

DNA is sensitive to low pH conditions and to elevated temperatures. Purine bases mainly are more prone to degradation than pyrimidine bases due to their low oxidation potential, and especially guanine nucleobase. For quantification of DNA following treatment exposure, comparison of UV and TIC traces from Mass spectrometry before and after treatment were used. The total DNA was quantified from UV area traces and the DNA quantification of each fragment was calculated from the TIC area traces. The fragments which do not appear on TIC and still visible on spectrum after deconvolution, were considered as detected.

Four buffers were selected based on their pH compatibility and usefulness in the DEL field: sodium acetate (pH 3.6 and pH 4.5), sodium phosphate (pH 5.5, 6.5 and 8), MOPS (pH 6.5, 7 and 7.5) and sodium borate (pH 8.5, 9.5 and 11) were used for investigating the durability.

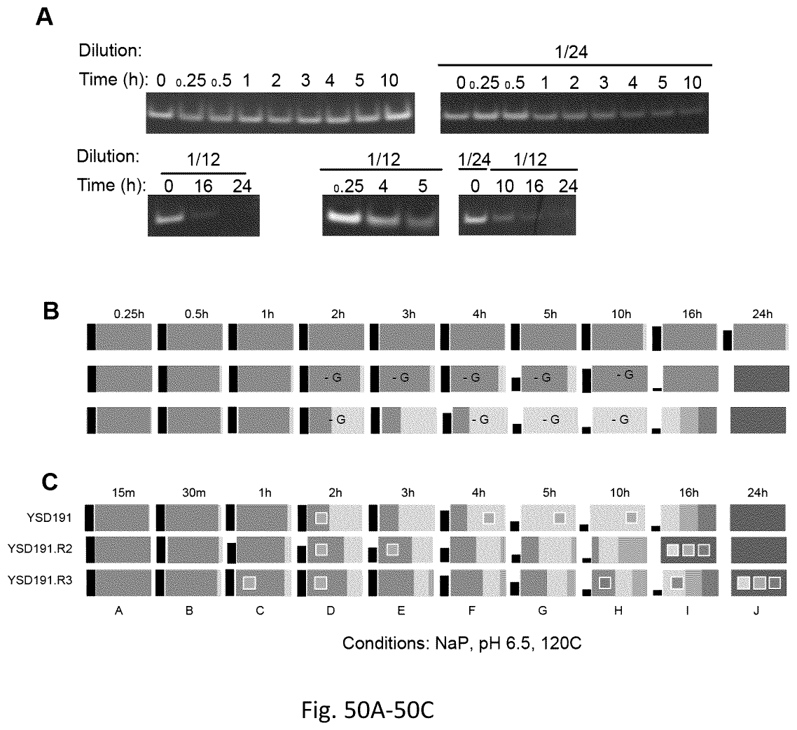

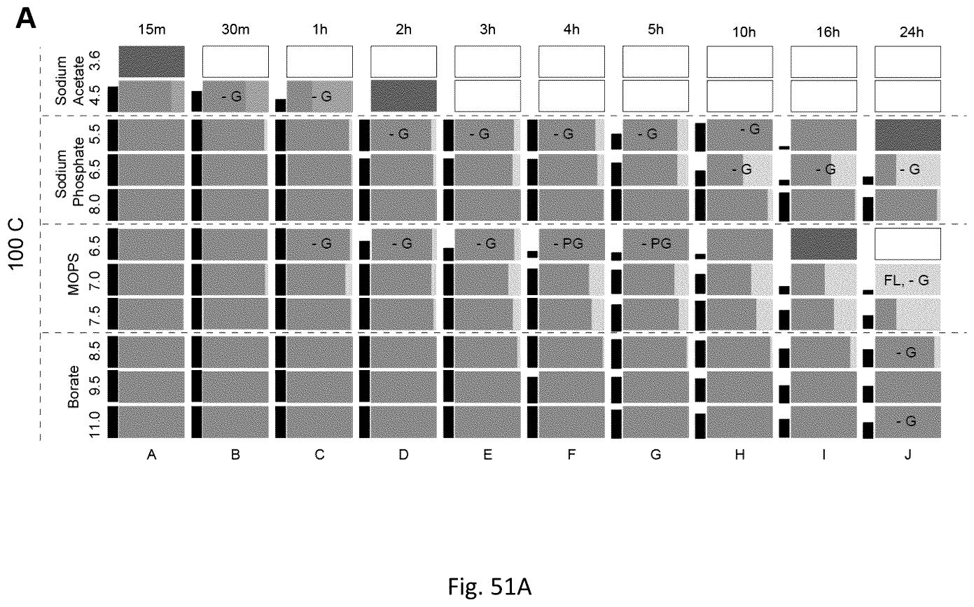

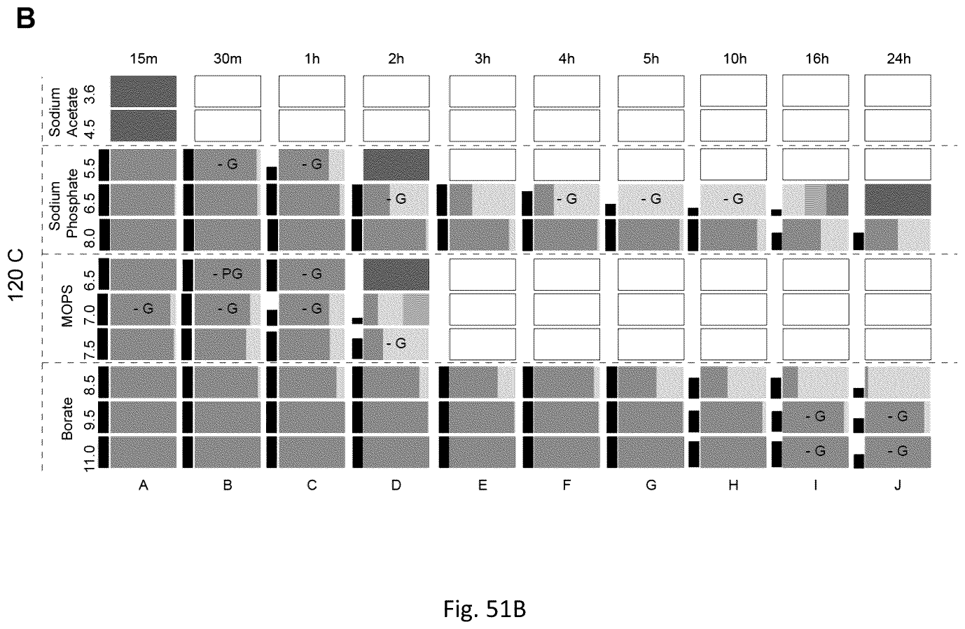

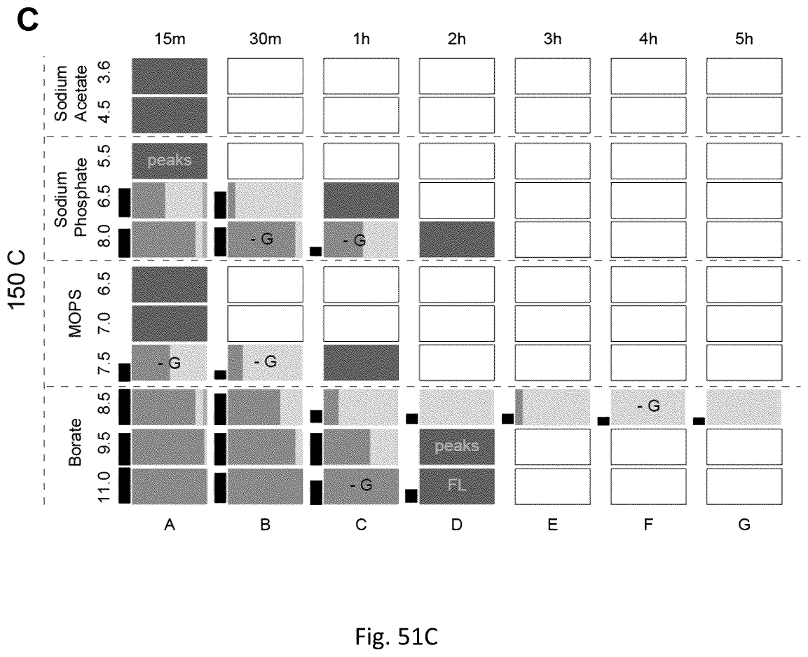

At 100° C., DNA tolerated well pH of 5.5-11 but at lower pH (3.6 and 4.5) a high level of degradation was visible (FIG. 51A, top 2 lines). Base removal was observed at pH 3.6 and 4.5 (blue, guanine removal). A systematic nucleobase removal (-G, -G-A, -G-G and -G-A-G) was observed at pH 3.6 after just 15 minutes (not shown). Tolerability was slightly better at pH 4.5 (green, FL observed up to 1 hour). In sodium phosphate buffer at pH 5.5 and 6.5 DNA was mostly stable for 4 hours with a little phosphate degradation starting after 30 minutes from 5′ end-nucleotide. DNA was fully degraded after 24 hours at pH 5.5 and 25% of total DNA remained at pH 6.5 (30% FL and 70%-Phos, yellow). In sodium phosphate buffer at pH 8.0, treatment was well tolerated (75% total DNA and mostly FL at 24 hours). DNA suffered similar degradation in MOPS buffer at pH 6.5, it was slightly better at pH 7 and pH 7.5 until 5 hours with 75% of total DNA. Basic conditions (pH >8.5) in sodium borate buffer DNA is very well tolerated (pH 8.5 to pH 11) with 50% of total DNA (mostly FL, green) after 24 hours. In summary, the results obtained at 100° C. were encouraging to investigate temperatures above 100° C., as higher temperatures are beneficial for organic chemistry.

At 120° C., significant differences were observed (FIG. 51B). DNA degradation was complete at lower pH (pH 3.6 and 4.5) in just 15 minutes. Interestingly, Phosphate buffer DNA was resisting with a total of 25% after 1 hour at pH 5.5 with almost 70% full length. Significant improvement was observed at pH 8.0 up to 10 hours with a total of 100% FL remaining, and 50% total DNA left after 24 hours (50% FL). DNA degradation was minimal in MOPS buffer up to 1 hour at pH 5.5. At 3 hours, in any pH condition, the DNA was fully degraded. At pH 8.5 and above in sodium borate borate, the durability of DNA was fairly good, and similarly tolerated at pH 9.5 and 11.0 with 50% intact DNA (FL) remaining after 24 hours. Because higher temperatures are preferable in organic chemistry and based on the encouraging results at 120° C., next 150° C. conditions were tested.

At 150° C., the amount of intact DNA (FL) left after an hour was very limited (FIG. 51C). Most DNA was degraded below pH 7.5 in as little as 15 minutes as depicted in FIG. 51C. Only proper DNA peaks were observed in basic conditions (pH >8.5) both in phosphate and borate buffers, and up to 1 hour.

Encouraged by previous results, and to generate a more precise map of DNA compatible conditions, a number of intermediary conditions were tested in sodium borate buffer, including longer time points (FIG. 52) and for 2 different pH (9.5 and 11.0). Remarkably, at 100° C. the DNA remained extremely stable with most of the DNA left being intact DNA (FL) (FIG. 52A). Similar results were obtained at 110° C. up to 72 hours for pH 9.5 and 48 hours for pH 11.0 (FIG. 52B). At 120° C., between one third and one half of the DNA remaining at 48 hours is intact DNA (FL) (FIG. 52C). At 130° C. and 140° C., interestingly, at the two pH tested, a significant amount of intact DNA (FL) remained up to 6 hours of incubation (FIG. 52 D-E).

### DNA Durability Based on Nucleotide Content.

Five different DNA sequences presenting an increasing GC content (0%, 25%, 50%, 75%, 100%), while keeping GCT as overhang sequence in all DNA fragments, were used. Five conditions based on previous results were selected: 1) sodium phosphate, pH 6.5, 150° C., 15 minutes, 2) sodium phosphate, pH 6.5, 120° C., 2 hours, 3) sodium borate pH 8.5, 120° C., 3 hours, 4) MOPS, pH 7.0, 100° C., 24 hours, and 5) MOPS, pH 7.0, 120° C., 2 hours.

Some significant differences were observed. In most conditions, except in borate buffer, a decrease of full-length DNA was observed in parallel with the increase of GC content. Depurination is likely the reason why acidic pH and higher temperatures induced degradation as GC content was increasing.

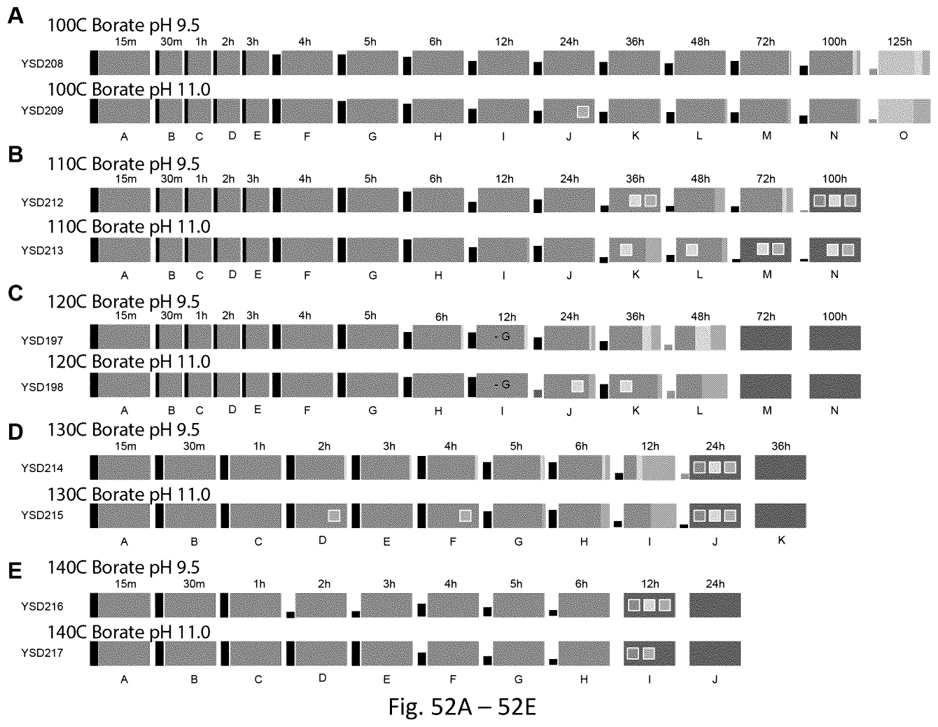

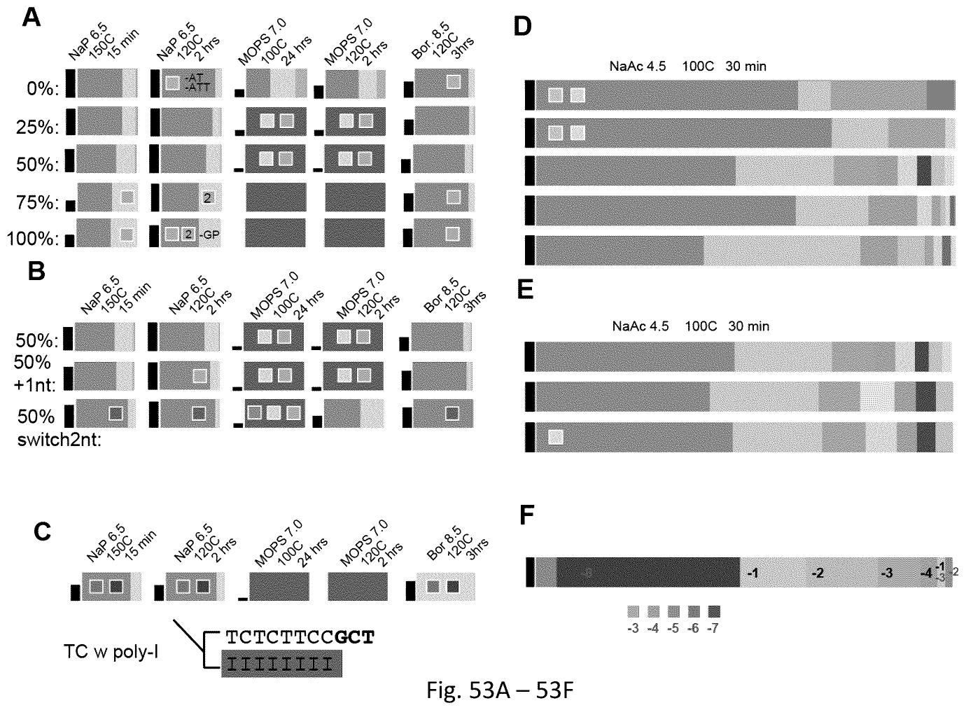

Because of high pH with borate buffer (FIG. 53A, right column) the results for all the sequences look similar independently of the GC content. Remarkably, at this temperature and pH, the total amount of full-length DNA is not affected by GC content, contrary to what is observed for other conditions (FIG. 53A, left column). Strikingly, the absence of GC nucleotides rendered the DNA much more resistant in two conditions (FIG. 53A, MOPS, top line) where any other DNA fragment containing one or more GC nucleotides led to total degradation (red rectangles). In these 0% GC conditions, depurination through adenine base was observed and full-length DNA was less affected by degradation when comparing to fragments containing GC nucleotides. In sodium phosphate buffer, an increase of dephosphorylation was observed in parallel with the increase of GC content. This was less obvious in sodium borate buffer. Instability due to high GC content is also visible at 75% and 100% GC (FIG. 53A, bottom 2 lines) especially for sodium phosphate buffer where -G, -G-G species were detected.

The results of nucleotide permutations did not affect DNA durability, unless a purine base was incorporated as the last 5′ nucleotide (FIG. 53B, bottom row). Indeed, this DNA fragment clearly showed that removing the last guanine nucleotide at the 5′ position, and replacing it with a thymine, increased DNA durability, both in terms of DNA quality and species present.

The approach also works well for non-DNA variants. To investigate this aspect, the nucleotide Inosine was used for an entire strand and made complementary to thymine nucleotides (FIG. 53C). The durability of the DNA containing Inosine nucleotides seemed less resistant than fragments containing natural nucleotides, as full-length DNA is reduced at least by half in all five conditions tested, and the loss of inosine nucleotide is easily observed (-I, -I-I).

Acidic conditions are typically not compatible with DNA, but for a number of chemical reactions, an acidic condition is necessary. As observed earlier, pH 4.5 was not as deleterious as pH 3.6, but it led to DNA degradation after an hour at 100° C. Therefore, various DNA properties were tested to investigate if some modifications could lead to higher DNA durability at pH 4.5 specifically. Surprisingly, many more species of modified DNA were found in these conditions. In this condition depurination occurred in all the DNA sequences (FIG. 53B, D, F). The depurination from the overhang nucleotide sequence GCT was observed along with the depurination of adenine nucleotides in the DNA fragments containing no GC nucleotides. Finally, regarding the inosine-thymine hybrid strand, the progressive removal of every single inosine nucleotide was observed as visualized by the brown gradient (FIG. 53F).

### Investigation of the Impact of Metal Catalysts on DNA Durability.

The results obtained so far at elevated temperatures in various buffers and at different pH encouraged us to investigate the impact of other key chemical parameters on DNA durability. In recent years, to advance the chemical diversity of the DEL space various methodologies have been developed. Transition-metal-catalysts-based reactions in the presence of DNA is one of the major areas that has been advancing in the DEL field. The predominant limitation of working with metal species in presence of DNA is the formation of Reactive Oxygen Species (ROS), that is dependent on the redox potential of each metal. ROS can result in DNA damages for metals presenting high redox potentials through oxidation of nucleobases or backbone breakage. Due to complexity of the pattern observed by LCMS, quantification was not prioritized, and results are presented as UV spectra and/or molecular ion peaks. Considering this, 9 types of metal catalysts were tested for their putative impact on DNA durability.

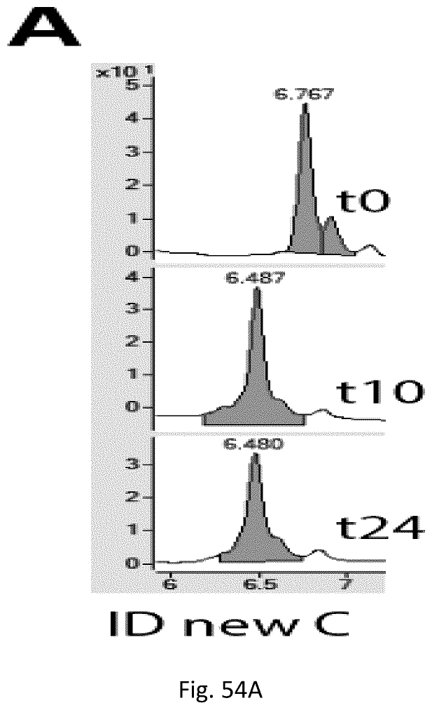

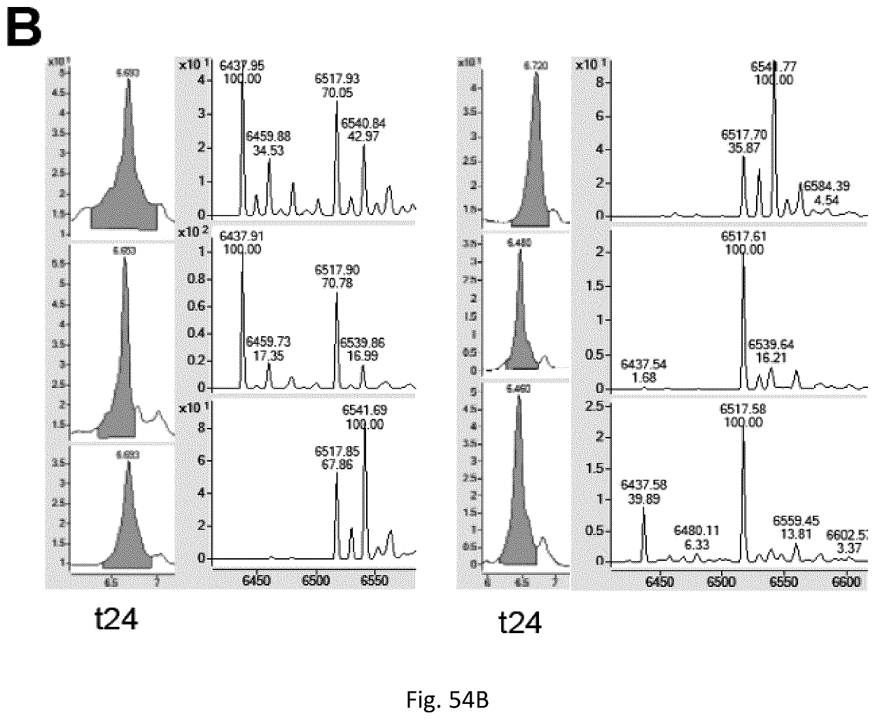

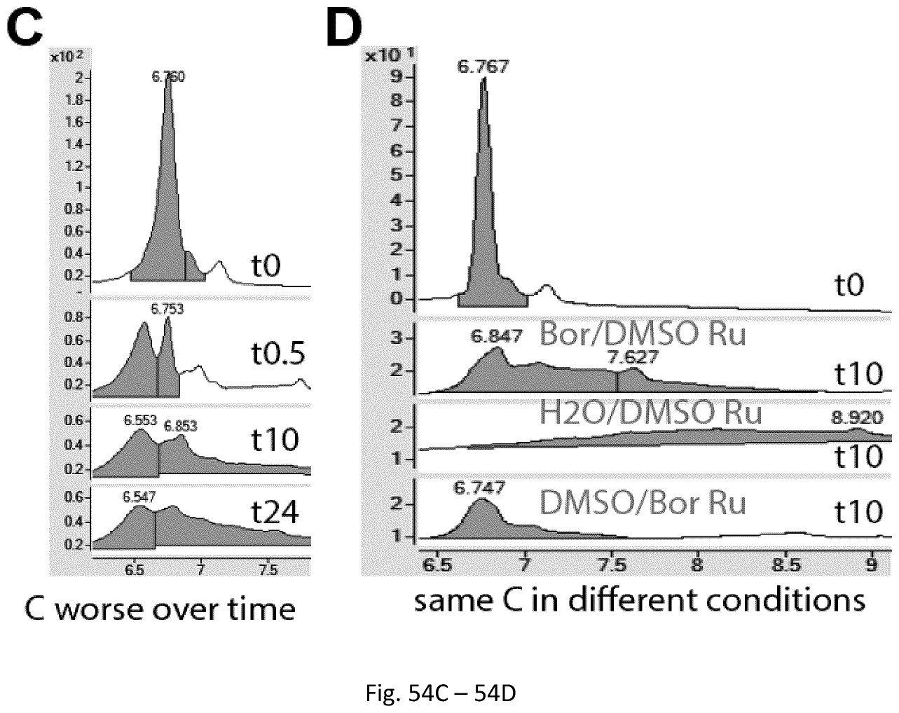

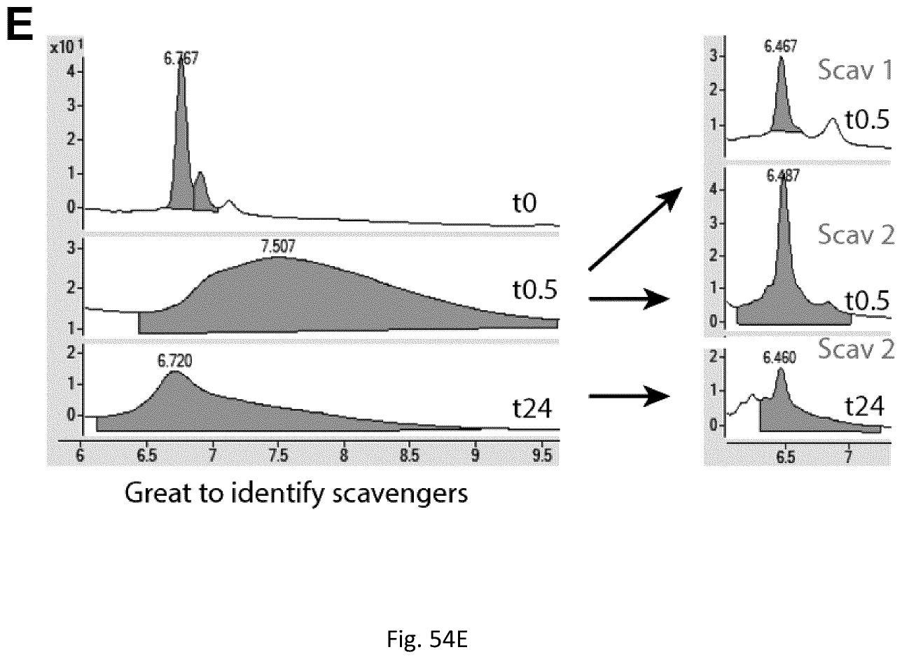

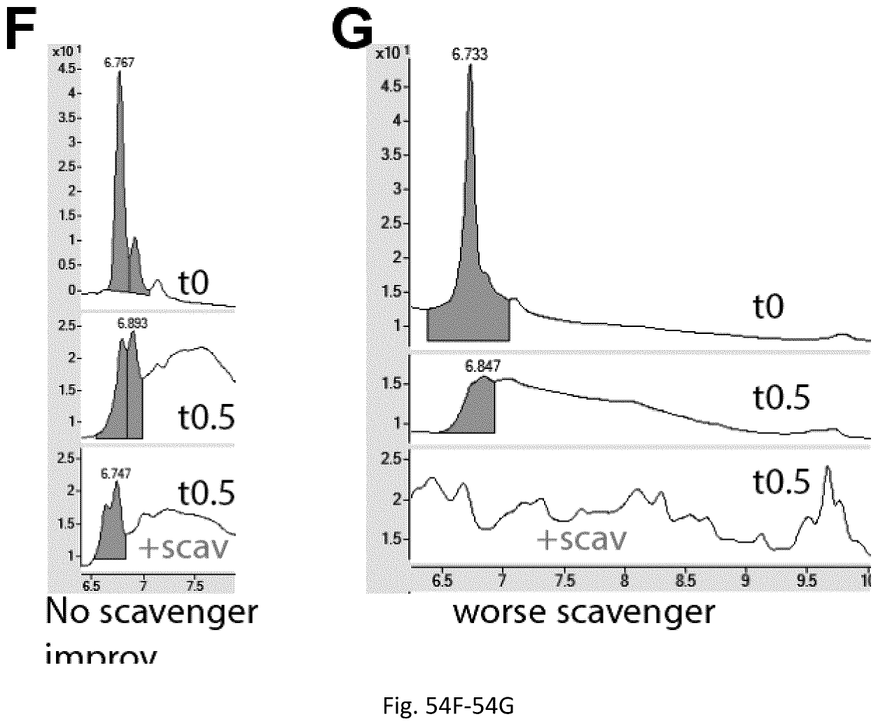

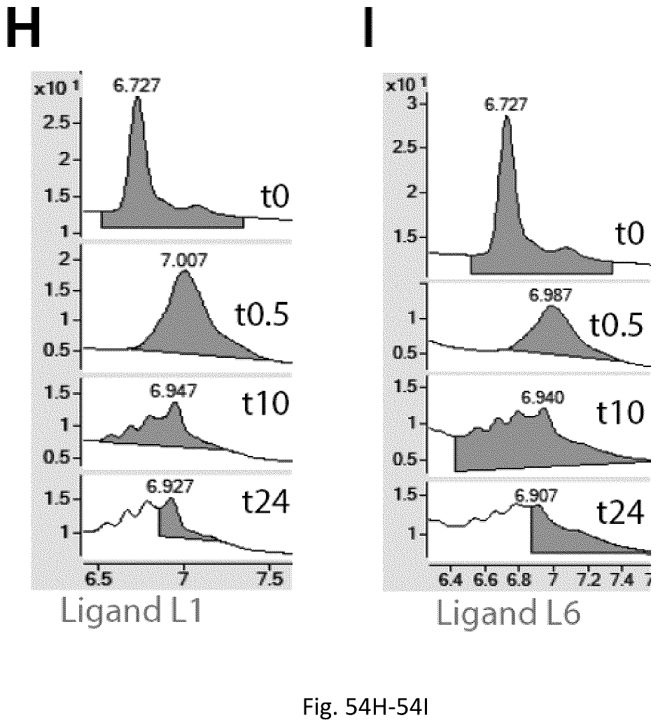

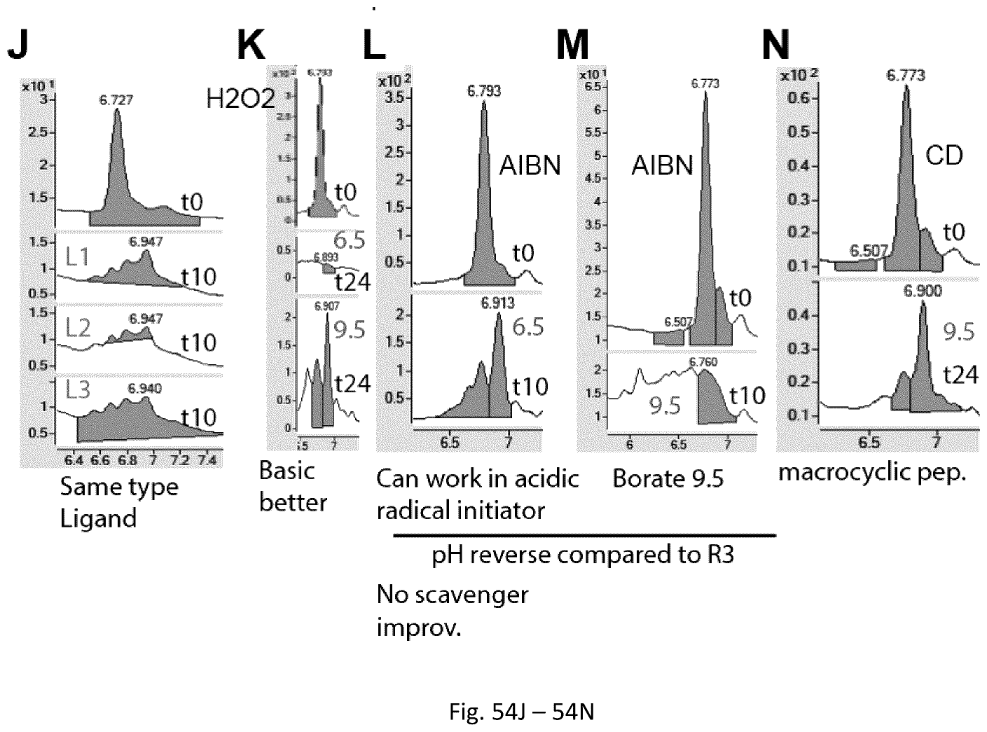

Four conditions were used to test metal catalysts at 100° C.: 1) phosphate buffer/DMSO (ratio 3:1) pH 6.5, 2) sodium borate/DMSO (ratio 3:1) pH 9.5, 3) H₂O/DMSO (ratio 3:1) and 4) DMSO/sodium borate (ratio 3:1) pH 9.5. All the catalysts were extemporarily prepared in DMSO at 10 mM and they were incubated in presence of DNA for 0.5, 10 and 24 hours, at 10 equivalents. A number of key observations were made and the main highlights are presented below. The results obtained can be divided into four groups. First, a group composed of five metal catalysts (Nickel (Ni), Cobalt (Co), Gold (Au), Silver (Ag) and Copper (Cu)). In the best cases, the catalysts did not affect DNA quality at 10 hours and up to 24 hours. A representative UV spectrum for Ag is presented in FIG. 54A. Based on the overall DNA quality, these five catalysts showed little DNA degradation as observed by LCMS at 24 hours in 6 conditions (FIG. 54B). In the case of Ni, adducts (possibly single atom) were observed at the two time points (sodium borate/DMSO)(not shown).

Second, other catalysts tested such as Ruthenium (Ru) are presenting a number of DNA quality issues in function of the conditions. Overall Ru did not cause severe DNA degradation, even though a time dependent effect can be seen (FIG. 54C). More clearly, the DNA quality post-treatment and the presence of adducts caused by Ru was very much dependent on the conditions; 3 conditions are presented as examples (FIG. 54D). In H₂O/DMSO, almost immediately (0.5 hours) adducts made the results totally uninterpretable (FIG. 54D, middle condition). Adducts were less present in sodium borate/DMSO, and barely detectable in DMSO/sodium borate (FIG. 54D). Silver was another catalyst that induced large DNA quality inequalities in function of the conditions.

Third, another interesting catalyst investigated is Palladium (Pd), a widely used metal catalyst in organic chemistry. Large amount of adducts, in all the conditions tested (except in phosphate buffer, not shown), starting after 30 minutes of incubation, were formed (FIG. 54E, 2^{nd} and 3^{rd} top graphs).

Fourth, Iridium (Ir) and rhodium (Rh(I) and Rh(III)) caused the formation of abundant adducts in all four conditions, with a slight improvement in Phosphate/DMSO (not shown). As expected, Rh(III) is not as good as Rh(I), most likely due to its higher oxidation state.

Even though dephosphorylation was observed in some conditions (mostly in H₂O/DMSO) it is unclear if these are due or amplified in presence of catalyst. It is important to note that Phosphate-based buffers have been behaving fairly well in these studies, and it is of particular interest, because both versions, acidic and basic, can be used.

Importantly, for all catalysts, one condition (Phosphate/DMSO) clearly emerged as more favorable in term of adduct formation. These adducts are typically caused by metal residues and scavenger molecules can be used to chelate those residues.

### Removal of Metal Residues Using Scavengers.

As observed above, adducts represent a limitation and in most cases render DNA detection impossible. Metal scavengers are used to chelate metal residues which leads to their removal from DNA. Various scavengers can be used depending on the nature of metal used. For example, Sulphur-based scavengers (e.g., Sodium diethyldithiocarbamate (NaDEDTC) and 2-Mercaptoethanol (BME)) can be used for soft metals removal. Oxygen-based scavengers (e.g., EDTA or Triaminetetraacetic acid (TAAcOH)) are effective for metals in low or zero oxidation states and their corresponding salts are more appropriate for higher oxidation state metals. In the present studies, NaDEDTC and BME were used.

Remarkably, a total recovery post-scavenger treatment was observed with both scavengers tested (NaDEDTC and BME) (FIG. 54E, right panels) even though palladium was one of the worst catalysts generating a complex set of adducts after just 30 minutes exposure (except in Phosphate/DMSO) as shown on FIG. 54E (left panel, t0.5 and t24). In H2O/DMSO, NaDEDTC and BME were effective at clearing Pd adducts but high level of DNA degradation was observed. In some conditions, the scavenger treatment did not help. Indeed, no significant improvement was observed for the catalysts Ru(II), Ir(I), Rh(III) post scavenger treatment and especially when these metals formed the most adducts (in H₂O/DMSO). An example for Ru(II) is shown in FIG. 54F. Adducts caused by Rh(I) were recovered well compare to Rh(III). Finally, in other conditions, the scavenger treatment might have further damage DNA as observed with NaDEDTC on Ir(I) (FIG. 54G).

In summary, scavenger treatments mostly recovered the DNA efficiently while differences were observed. More importantly, this confirms this approach as a well suited one to identify novel metal scavengers, efficient and DNA-safe.

### Investigation of the Impact of Chemical Ligands and Other Reagents on DNA Durability.

Six metal ligands and six reagents were used to investigate DNA durability. All metal ligands and reagents were used at 100 equivalents at 100° C. The phosphine-based ligands Xantphos, tBuBrettPhos and DPEphos were well tolerated. For all three ligands, no adducts were observed at 30 minutes in Phosphate/DMA (ratio 4:1), minimal amounts were observed at 10 hours and slightly more at 24 hours (not shown). In sodium borate buffer at 10 hours these phosphine-based ligands caused DNA degradation and even more so at 24 hours (FIG. 54, H-J). The simpler phosphine ligand Triphenylphosphine was well tolerated up to 24 hours in both phosphate and borate buffers. The ligands 1, 10-Phenathroline and Triphenylbismut were well tolerated in Phosphate buffer up to 24 hours. Whereas in sodium borate buffer at 24 hours both were causing higher levels of degradation.

Two oxidizing reagents were used in this study (H₂O₂ and 2-Iodoxybenzoic acid (IBX)). H₂O₂ affected DNA quality substantially in Phosphate buffer, most likely because of the acidic condition (pH 6.5) used. At the contrary, in basic condition H₂O₂ incubation was better tolerated with only minimal degradation over time (FIG. 54K, pH 6.5 vs. 9.5). Notably, DNA species missing one guanine nucleotide was observed. IBX was well tolerated in both acidic and basic conditions.

Next, two radical based reagents were used: Azobisisobutyronitrile (AIBN) and 2,2,6,6-Tetramethylpiperidin-1-yl-oxyl (TEMPO). The radical initiator AIBN was well tolerated in acidic conditions (pH6.5) whereas in basic conditions (pH 9.5) it caused significant DNA degradation (FIG. 54L, M). TEMPO was well tolerated in both acidic and basic conditions but a higher dephosphorylation was detected at pH 6.5 (not shown).

A cyclic oligosaccharide macrocycle (Cyclodextrin (CD)) was well tolerated in both acidic and basic conditions (FIG. 54N).

Finally, the base cesium hydroxide (CsOH) used at 500 eq. was well tolerated in both acidic and basic conditions. However, DNA dephosphorylation due to the acidic pH was not neutralized by the base CsOH even at higher equivalence (not shown).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A system for identifying an oligonucleotide having high ligation efficiency, the system comprising:
a) a substrate for attaching a first accessory oligonucleotide;
b) a first accessory oligonucleotide comprising a moiety for attaching the first oligonucleotide to the substrate;
c) a second accessory oligonucleotide comprising a moiety for recognition by a detectable moiety;
d) a detectable moiety for detection of the ligation between the first accessory oligonucleotide, an intermediary test oligonucleotide and the second accessory oligonucleotide; and
e) at least one intermediary test oligonucleotide.

2. The system of claim 1, wherein the first accessory oligonucleotide is conjugated to biotin.

3. The system of claim 1, wherein the second accessory oligonucleotide is conjugated to digoxigenin (DIG).

4. The system of claim 1, wherein the detectable moiety comprises an anti-DIG-HRP antibody.

5. The system of claim 1, wherein the intermediary test oligonucleotide is selected from the group consisting of a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, and an oligonucleotide comprising or encoding sequences for antibody recognition.

6. A system for identifying an oligonucleotide having high ligation efficiency, the system comprising:
a) a substrate for attaching a first accessory oligonucleotide;
b) an accessory oligonucleotide comprising a moiety for attaching the accessory oligonucleotide to the substrate;
c) a test nucleic acid molecule comprising a moiety for recognition by a detectable moiety; and
d) a detectable moiety for detection of the ligation between the accessory oligonucleotide, and the test nucleic acid molecule.

7. The system of claim 6, wherein the accessory oligonucleotide is conjugated to biotin.

8. The system of claim 6, wherein the test nucleic acid molecule is conjugated to digoxigenin (DIG).

9. The system of claim 6, wherein the detectable moiety comprises an anti-DIG-Horse Radish Peroxidase (HRP) antibody.

10. The system of claim 6, wherein the test nucleic acid molecule is selected from the group consisting of a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, an oligonucleotide comprising or encoding sequences for antibody recognition, a nucleic acid molecule conjugated to a molecule for binding or purification, a nucleic acid-antibody conjugate, a nucleic acid-nanobody conjugate, a nucleic acid molecule conjugated to a molecule for visulaization, a nucleic acid-fluorophor conjugate, a nucleic acid-quantum dot conjugate, a nucleic acid molecule conjugated to a molecule for cell permeabilization, and a nucleic acid molecule conjugated to a molecule for proximal PCR or rolling circle amplification (RCA).

11. A method for identifying an oligonucleotide having high ligation efficiency, the method comprising the steps of:
a) contacting an intermediary test oligonucleotide molecule with a first accessory oligonucleotide conjugated to a surface and a second accessory oligonucleotide conjugated to a moiety for direct or indirect detection,
b) ligating the intermediary test oligonucleotide molecule to the first accessory oligonucleotide and the second accessory oligonucleotide,
c) removing any unligated oligonucleotides by washing,
d) contacting the ligated complexes with a detectable molecule for colorimetric, chemiluminescent or fluorescent detection, and
e) determining the ligation efficiency of the test oligonucleotide molecule to the first accessory oligonucleotide and the second accessory oligonucleotide by detecting the colorimetric, luminescencent or fluorescencent readout of the detectable molecule.

12. The method of claim 11, wherein the first accessory oligonucleotide is conjugated to biotin.

13. The method of claim 11, wherein the second accessory oligonucleotide is conjugated to digoxigenin.

14. The method of claim 11, wherein the detectable molecule comprises an anti-DIG-HRP antibody.

15. The method of claim 11, wherein the intermediary test oligonucleotide is selected from the group consisting of a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, and an oligonucleotide comprising or encoding sequences for antibody recognition.

16. A method for identifying an oligonucleotide having high ligation efficiency, the method comprising the steps of:
a) contacting an test nucleic acid molecule with an accessory oligonucleotide conjugated to a surface,
b) ligating the test nucleic acid molecule to the accessory oligonucleotide,
c) removing any unligated oligonucleotides by washing,
d) contacting the ligated complexes with a detectable molecule for colorimetric, chemiluminescent or fluorescent detection, and
e) determining the ligation efficiency of the test nucleic acid molecule to the accessory oligonucleotide molecule by detecting the colorimetric, luminescencent or fluorescencent readout of the detectable molecule.

17. The method of claim 16, wherein the accessory oligonucleotide is conjugated to biotin.

18. The method of claim 16, wherein test nucleic acid molecule is conjugated to digoxigenin.

19. The method of claim 16, wherein the detectable molecule comprises an anti-DIG-HRP antibody.

20. The method of claim 16, wherein the test nucleic acid molecule is selected from the group consisting of a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, an oligonucleotide comprising or encoding sequences for antibody recognition, a nucleic acid molecule conjugated to a molecule for binding or purification, a nucleic acid-antibody conjugate, a nucleic acid-nanobody conjugate, a nucleic acid molecule conjugated to a molecule for visulaization, a nucleic acid-fluorophor conjugate, a nucleic acid-quantum dot conjugate, a nucleic acid molecule conjugated to a molecule for cell permeabilization, and a nucleic acid molecule conjugated to a molecule for proximal PCR or rolling circle amplification (RCA).

21. A method of tagging a DNA encoded library (DEL), the method comprising ligating at least one oligonucleotide molecule to the DEL.

22. The method of claim 21, wherein the oligonucleotide molecule comprises a DNA blocker molecule comprising a restriction enzyme recognition site and a free chemical group.

23. The method of claim 22, wherein the method further comprises contacting the tagged DEL with a restriction enzyme for cleaving the tagged DEL at the restriction enzyme recognition site and subsequently ligating a at least one additional oligonucleotide molecule to the DEL.

24. The method of claim 23, wherein the at least one additional oligonucleotide molecule is selected from the group consisting of a DNA fragment, a non-natural DNA fragment, a locked nucleic acid molecule, a tagging oligonucleotide, a barcode oligonucleotide, a spacer oligonucleotide, an oligonucleotides comprising a restriction enzyme recognition site, an oligonucleotide comprising or encoding sequences for antibody recognition.

25. The method of claim 24, wherein at least one additional oligonucleotide molecule is a DNA blocker molecule comprising a restriction enzyme recognition site and a free chemical group.

26. The method of claim 22, further comprising functionalizing the DNA blocker molecule with a molecule to increase solubility in organic solvents.

27. An oligonucleotide tag molecule comprising at least one nucleotide sequence selected from the group consisting of: TCGGAGAA, TCGGAGCT, TCGGATAC, TCGGACGT, TCGGAGAT, TCGGAAGA, TCGGATTG, TCGGACAA, TCGGAGTA, TCGGAAGT, TCGGATCA, TCGGACAT, TCGGAGTT, TCGGAACA, TCGGATCT, TCGGACTA, TCGGAGCA, TCGGAACT, TCGGACGA, TCGGACTT, CAGAGGAG, CAGAGGTA, CAGAGAGG, CAGAGAAC, CAGAGGAA, CAGAGGTT, CAGAGAGA, CAGAGATG, CAGAGGAT, CAGAGGTC, CAGAGAGT, CAGAGATC, CAGAGGAC, CAGAGGCA, CAGAGAGC, CAGAGACG, CAGAGGTG, CAGAGGCT, CAGAGAAG, CAGAGACA, ACGTGGAG, ACGTGGTA, ACGTGAGG, ACGTGAAC, ACGTGGAA, ACGTGGTT, ACGTGAGA, ACGTGATG, ACGTGGAT, ACGTGGTC, ACGTGAGT, ACGTGATC, ACGTGGAC, ACGTGGCA, ACGTGAGC, ACGTGACG, ACGTGGTG, ACGTGGCT, ACGTGAAG, and ACGTGACA.

28. A kit comprising at least one of:
a) a substrate for attaching a first accessory oligonucleotide; and
b) a first accessory oligonucleotide comprising a moiety for attaching the first accessory oligonucleotide to the substrate.

29. The kit of claim 28 further comprising
c) a second accessory oligonucleotide comprising a moiety for recognition by a detectable moiety; and
d) a detectable moiety for detection of the ligation between the first accessory oligonucleotide, an intermediary test oligonucleotide and the second accessory oligonucleotide.

30. A DNA solubilizer molecule comprising a DNA blocker molecule comprising a restriction enzyme recognition site covalently linked to a molecule for modulating the solubility of DNA.

31. The DNA solubilizer molecule of claim 30, wherein the molecule for modulating the solubility of DNA is selected from the group consisting of polyethylene glycol (PEG), methoxy PEG-succinimidyl carboxyl methyl ester, and methoxy PEG-succinimidyl carboxyl methyl ester with a N-Hydroxysuccinimide group.

32. The DNA solubilizer molecule of claim 30, wherein the molecule for modulating the solubility of DNA comprises methoxy PEG-succinimidyl carboxyl methyl ester having a molecular weight selected from 500 to 50,000.

33. A method of identifying DNA solubilizer molecules that can alter the solubility of DNA molecules and DEL, the method comprising:
a) ligating a DEL DNA molecule, or a DNA molecule to be ligated to a DEL, to a DNA solubilizer molecule of any one of claims 30-32,
b) contacting the fused DEL DNA molecule:DNA solubilizer molecule with an organic solvent and testing the solubility of the fused DEL DNA molecule:DNA solubilizer molecule in the organic solvent.

34. The method of claim 33, wherein the organic solvent is selected from the group consisting of DMSO, DMF, DMA, 1,4-dioxane, ACN and DCM.

35. A method of generating a DEL containing compounds that require an organic solvent, the method comprising:
a) ligating a DEL DNA molecule to a DNA solubilizer molecule of any one of claims 30-32,
b) contacting the ligated DEL DNA molecule:DNA solubilizer molecule with the organic solvent,
c) performing at least one chemical reaction to attach the DEL DNA molecule to the compound that requires an organic solvent, and
d) contacting the ligated DEL DNA molecule:DNA solubilizer molecule with a restriction enzyme to remove the DNA solubilizer molecule, thereby allowing for further ligation and labeling of the DEL DNA molecule.

36. The method of claim 35, wherein the organic solvent is selected from the group consisting of DMSO, DMF, DMA, 1,4-dioxane, ACN and DCM.

37. A DEL generated according to the method of claim 35.

38. A method of identifying reagents or conditions that can alter the durability of DNA molecules and DEL, the method comprising:
a) obtaining a short double stranded oligonucleotide molecule to be tested,
b) covalently linking the two strands of the short oligonucleotide molecule to be tested with a spacer that carries a free functional group for chemical addition and denaturing the linked double stranded oligonucleotide molecule to generate a linked ssDNA molecule,
c) contacting the linked ssDNA molecule of a) with one or more condition or reagent to be tested for its effect on DNA durability, and
d) analyzing the effect of the condition or reagent on the durability of the short oligonucleotide molecule.

39. The method of claim 38, wherein the condition or reagent to be tested is selected from the group consisting of organic solvents, buffers, high temperatures, altered pH, metal catalysts, metal scavengers, % GC content, non-natural nucleotides, chemical ligands and any combination thereof.

40. The method of claim 38, wherein the method further includes a step of precipitating the short oligonucleotide molecule prior to analyzing the effect of the condition or reagent on the durability of the molecule.

41. The method of claim 38, wherein the method of analyzing the effect of the condition or reagent on the durability of the short oligonucleotide molecule is performed using gel electroporation, LCMS, or a combination thereof.

42. A DEL generated in a condition identified according to the method of any one of claim 38-41 as increasing DNA durability.


SEQUENCE LISTING (30 sequences)

SEQ ID NO: 1 - DNA, 13 bp, synthetic construct
  source 1..13: mol_type = other DNA
gttacagatcagc

SEQ ID NO: 2 - DNA, 48 bp, synthetic construct
  source 1..48: mol_type = other DNA
ggtggattaattaaagtgacactagcaatcgtgctgatctgtaacgct

SEQ ID NO: 3 - DNA, 13 bp, synthetic construct
  source 1..13: mol_type = other DNA
gttacagatcagc

SEQ ID NO: 4 - DNA, 48 bp, synthetic construct
  source 1..48: mol_type = other DNA
ggtggagtttaaactgatgctcaagcaatcgtgctgatctgtaacgct

SEQ ID NO: 5 - DNA, 13 bp, synthetic construct
  source 1..13: mol_type = other DNA
gttacagatcagc

SEQ ID NO: 6 - DNA, 54 bp, synthetic construct
  source 1..54: mol_type = other DNA
ggtggaggccgtcagaggccactagtgtcatcaatcgtgctgatctgtaacgct

SEQ ID NO: 7 - DNA, 13 bp, synthetic construct
  source 1..13: mol_type = other DNA
gttacagatcagc

SEQ ID NO: 8 - DNA, 48 bp, synthetic construct
  source 1..48: mol_type = other DNA
ggtggaggcgcgccagaccagtcagacatcgtgctgatctgtaacgct

SEQ ID NO: 9 - DNA, 13 bp, synthetic construct
  source 1..13: mol_type = other DNA
gttacagatcagc

SEQ ID NO: 10 - DNA, 46 bp, synthetic construct
  source 1..46: mol_type = other DNA
ggtggagaattcgatccagcgatgtatcgtgctgatctgtaacgct

SEQ ID NO: 11 - DNA, 13 bp, synthetic construct
  source 1..13: mol_type = other DNA
gttacagatcagc

SEQ ID NO: 12 - DNA, 46 bp, synthetic construct
  source 1..46: mol_type = other DNA
ggtggaaagcttgtacgtcatcgctatcgtgctgatctgtaacgct

SEQ ID NO: 13 - DNA, 13 bp, synthetic construct
  source 1..13: mol_type = other DNA
gttacagatcagc

SEQ ID NO: 14 - DNA, 52 bp, synthetic construct
  source 1..52: mol_type = other DNA
ggtggacctcgagagaccactgcagtcatcaatcgtgctgatctgtaacgct

SEQ ID NO: 15 - DNA, 27 bp, synthetic construct
  source 1..27: mol_type = other DNA
taacatcgatcgatagctagctaatcg

SEQ ID NO: 16 - DNA, 29 bp, synthetic construct
  source 1..29: mol_type = other DNA
cgattagctagctatcgatcgatgttaat

SEQ ID NO: 17 - DNA, 27 bp, synthetic construct
  source 1..27: mol_type = other DNA
aattcatcggagctgcaggtcagcatc

SEQ ID NO: 18 - DNA, 23 bp, synthetic construct
  source 1..23: mol_type = other DNA
gatgctgacctgcagctccgatg

SEQ ID NO: 19 - DNA, 10 bp, synthetic construct
  source 1..10: mol_type = other DNA
  modified_base 1: mod_base = OTHER; note = locked nucleic acid
  modified_base 4: mod_base = OTHER; note = locked nucleic acid
  modified_base 6: mod_base = OTHER; note = locked nucleic acid
gactatccag

SEQ ID NO: 20 - DNA, 10 bp, synthetic construct
  source 1..10: mol_type = other DNA
gatagtccga

SEQ ID NO: 21 - DNA, 10 bp, synthetic construct
  source 1..10: mol_type = other DNA
  modified_base 1: mod_base = OTHER; note = locked nucleic acid
  modified_base 4: mod_base = OTHER; note = locked nucleic acid
  modified_base 6: mod_base = OTHER; note = locked nucleic acid
gtgagttcag

SEQ ID NO: 22 - DNA, 10 bp, synthetic construct
  source 1..10: mol_type = other DNA
aactcaccga

SEQ ID NO: 23 - DNA, 10 bp, synthetic construct
  source 1..10: mol_type = other DNA
  modified_base 1..7: mod_base = OTHER; note = phosphorothioate
gactatccag

SEQ ID NO: 24 - DNA, 10 bp, synthetic construct
  source 1..10: mol_type = other DNA
  modified_base 1..7: mod_base = OTHER; note = phosphorothioate
gatagtccga

SEQ ID NO: 25 - DNA, 10 bp, synthetic construct
  source 1..10: mol_type = other DNA
  modified_base 1..7: mod_base = OTHER; note = phosphorothioate
gtgagttcag

SEQ ID NO: 26 - DNA, 10 bp, synthetic construct
  source 1..10: mol_type = other DNA
  modified_base 1..7: mod_base = OTHER; note = phosphorothioate
aactcaccga

SEQ ID NO: 27 - DNA, 10 bp, synthetic construct
  source 1..10: mol_type = other DNA
  modified_base 2: mod_base = OTHER; note = i2F flurorine
  modified_base 5: mod_base = OTHER; note = i2F flurorine
  modified_base 7: mod_base = OTHER; note = i2F flurorine
gactatccag

SEQ ID NO: 28 - DNA, 10 bp, synthetic construct
  source 1..10: mol_type = other DNA
gatagtccga

SEQ ID NO: 29 - DNA, 10 bp, synthetic construct
  source 1..10: mol_type = other DNA
  modified_base 2: mod_base = OTHER; note = i2F flurorine
  modified_base 5: mod_base = OTHER; note = i2F flurorine
  modified_base 7: mod_base = OTHER; note = i2F flurorine
gtgagttcag

SEQ ID NO: 30 - DNA, 10 bp, synthetic construct
  source 1..10: mol_type = other DNA
aactcaccga